(19) United States
(12) Patent Application Publication
(10) Pub. No.: US 2024/0269194 A1
(43) Pub. Date: Aug. 15, 2024

(54) COMPOSITIONS AND METHODS FOR ISLET CELL TRANSPLANTS

(71) Applicant: City of Hope, Duarte, CA (US)

(72) Inventor: Fouad Kandeel, La Palma, CA (US)

(21) Appl. No.: 18/567,094

(22) PCT Filed: Jun. 9, 2022
(86) PCT No.: PCT/US2022/032878

§ 371 Date: Dec. 5, 2023

Related U.S. Application Data
(60) Provisional application No. 63/209,236, filed on Jun. 10, 2021.

(51) Int. Cl. A61K 35/39; A61K 9/00; A61K 31/4439; A61K 31/4985; A61K 38/22; A61P 3/10; C12N 5/071

(52) U.S. Cl. CPC A61K 35/39; A61K 9/0019; A61K 31/4439; A61K 31/4985; A61K 38/2207; A61P 3/10; C12N 5/0676

(57) ABSTRACT

Provided herein, inter alia, are compositions and methods for treating diabetes in a subject in need thereof. The methods include administering to the subject gastrin-treated islet cells.

### CROSS-REFERENCE TO RELATED APPLICATION

This application claims the benefit of and priority to U.S. Provisional Application No. 63/209,236 filed on Jun. 10, 2021, the contents of which are incorporated herein by reference in its entirety.

### SEQUENCE LISTING

The instant application contains a Sequence Listing which has been submitted via EFS-Web. The content of the text file named “048440-791001WO_ST25.txt”, which was created on Jun. 8, 2022 and is 1,499 bytes in size, is hereby incorporated by reference in its entirety.

### BACKGROUND OF THE INVENTION

In the U. S., diabetes is the seventh leading cause of mortality (1), and the American Diabetes Association estimated that in 2018, there were 34.2 million Americans who had diabetes. Additionally, in the U.S., the direct cost of diagnosed diabetes in 2017 was around $327 billion (2).

The prevalence of diabetes among American veterans is higher than in the general population (10.5%); veterans make up 9% of the general population, but approximately 25% of veterans are diabetic (3) due to the high incidence of obesity among them (3). Another potential contributing factor is alcohol abuse (4-6). Veterans were more likely to drink alcohol than civilians and to report heavy alcohol use (7). Hypoglycemia and chronic pancreatitis are frequent complications of abusing alcohol (8), and chronic pancreatitis leads to the death of insulin-producing beta cells and type 1 and 2 diabetes (9, 10).

The incidence of type 2 diabetes (T2D) has reached epidemic proportions, with 1 out of 3 children born in USA in year 2000 projected to develop diabetes within their lifetime. Despite advances in diabetes therapies and technology, achieving and maintaining glycemic targets remains challenging for most patients, increasing the risk of developing debilitating cardiovascular complications and reducing life expectancy. Prolonged exposure to hyperglycemia results in islet inflammation, beta cell dedifferentiation and reduced insulin secretion, which make managing T2D progressively more difficult.

Type 1 diabetes (T1D) is a chronic progressive disease requiring life-long treatment. In 2020, there will be about 1.6 million adults and children with type 1 diabetes (T1D) in the U. S. T1D individuals are at risk of developing serious complications that shorten their life expectancy by 11-13 years (11). T1D results from autoimmune destruction of insulin-producing beta cells within the pancreatic Islets of Langerhans. The disease is associated with unstable blood glucose and acute and long-term complications, such as hypoglycemia and hypoglycemia unawareness, which persist in many patients despite recent advances in insulin delivery and continuous glucose monitoring devices (12).

Islet transplantation (IT) effectively resolves severe hypoglycemia, improves overall glycemic control, and sometimes leads to insulin independence in T1D individuals. Modifications in immune suppression including use of T-cell depleting (e.g. anti-thymoglobulin) and anti-inflammatory agents (e.g. etanercept) have improved IT outcomes (13) (14). Nevertheless, many IT recipients continue to require islets from multiple donors and islet graft function tends to decline over time due transplant of inadequate islet mass leading beta cell exhaustion, allorejection or autoimmune reactivation. The shortage of deceased donor pancreata represents a barrier to the widespread use of IT. Strategies to protect and stimulate islet cell expansion and function would enhance the effectiveness of IT and are needed to expand access to this beneficial life-changing therapy.

Provided herein, inter alia, are solutions to these and other problems in the art.

### BRIEF SUMMARY OF THE INVENTION

In an aspect is provided a method of treating diabetes in a subject in need thereof, the method including administering a dosage of gastrin-treated human islet cells to the subject, wherein the dosage includes less than 9,000 IEQ/kg of islet cells. In some embodiments, the dosage comprises less than 8,000 IEQ/kg of islet cells. In some embodiments, the dosage comprises less than 7,000 IEQ/kg of islet cells. In some embodiments, the dosage comprises less than 6,000 IEQ/kg of islet cells. In some embodiments, the dosage comprises less than 5,000 IEQ/kg of islet cells. In the present disclosure, a “dosage” may refers to a pharmaceutically effective dosage or amount of a molecule (e.g., gastrin) useful for treatment, prevention, or amelioration of a disease or disorder described herein (e.g., diabetes), or capable of treating, preventing, or ameliorating at least one symptom of a disease or disorder described herein (e.g., diabetes). Such dosage can be determined by a doctor for each of patients.

In some embodiments, the gastrin-treated human islet cells are treated with gastrin or a gastrin variant or homologs. For example, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gastrin protein. In embodiments, the gastrin protein is substantially identical to the protein identified by the UniProt reference number P01350 or a variant or homolog having substantial identity thereto. In embodiments, the gastrin variant is gastrin-34, gastrin-17 or gastrin-14. In embodiments, the gastrin variant is gastrin-17. In embodiments, gastrin-17 includes the amino acid sequence Pyr-GPWLEEEEEAYGWMDF-NH2 (SEQ ID NO: 1). In embodiments, gastrin-17 is at least 80%, 85%, 90%, 95%, or 99% homologous or identical to the amino acid sequence of SEQ ID NO: 1. In embodiments, the gastrin variant is an analog of gastrin-17. In embodiments, the gastrin-17 analog is [Leu¹⁵] Gastrin-17 (GAST-17). In some embodiments, the gastrin-treated human islet cells are treated with gastrin 17. Gastrin may be a naturally occurring gastrin protein or a gastrin variant or homologs, as described herein, or a polynucleotide encoding a naturally occurring gastrin protein or a gastrin variant or homologs, as described herein. In some embodiments, the gastrin comprises a polypeptide having an amino acid sequence having at least 70%, 75%, 80%, 85%, 90%, 95%, 96%, 97%, 98%, 99%, or more, identity to SEQ ID NO: 1 or 2. In some embodiments, the gastrin comprises a polynucleotide encoding a polypeptide having an amino acid sequence having at least 70%, 75%, 80%, 85%, 90%, 95%, 96%, 97%, 98%, 99%, or more, identity to SEQ ID NO: 1 or 2.

In some embodiments, the human islet cells are obtained from the subject. In some embodiments, the human islet cells are not obtained from the subject. In some embodiments, the gastrin-treated human islet cells are obtained by a method comprising:
culturing islet cells from a donor;
contacting the culture with gastrin; and,
harvesting the islet cells.

In some embodiments, the method further comprises administering to the subject gastrin. Such gastrin may be a naturally occurring gastrin protein or a gastrin variant or homologs, as described herein, or a polynucleotide encoding a naturally occurring gastrin protein or a gastrin variant or homologs, as described herein.

In some embodiments, the gastrin is administered to the subject prior to administration of the dosage of the gastrin-treated human islet cells. In some embodiments, the gastrin is administered to the subject after the administration of the dosage of gastrin-treated human islet cells. In some embodiments, the gastrin is administered to the subject about two days after the administration of the dosage of gastrin-treated human islet cells.

In some embodiments, the gastrin is administered to the subject at least one time per day for about 30 days. In some embodiments, the gastrin is administered to the subject two times per day.

In some embodiments, the gastrin is administered to the subject about two days after the administration of the dosage of gastrin-treated human islet cells for two times per day for about 30 days.

In some embodiments, the gastrin is administered to the subject at a dosage of about 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or more. In some embodiments, the gastrin is administered to the subject at a dosage of about 15 μg/kg. These dosage may be administered at least once per day. In some embodiments, the dosage described herein is administered two times per day.

In some embodiments, the gastrin is administered to the subject subcutaneously, intramuscularly, intravenously, intrathecal, or any combination thereof. In some embodiments, the gastrin is administered to the subject subcutaneously.

In some embodiments, the method further comprises administering a second dosage of gastrin to the subject. In some embodiments, the second dosage of gastrin is administered to the subject about six months after administering the dosage of gastrin-treated human islet cells. In some embodiments, the second dosage of gastrin is administered to the subject is at least one time per day for about 30 days. In some embodiments, the second dosage of gastrin is administered to the subject two times per day.

In some embodiments, the method further comprises administering to the subject a proton pump inhibitor and a DPP-4 inhibitor. In some embodiments, the proton pump inhibitor is Esomeprazole. In some embodiments, the DPP-4 inhibitor is Sitagliptin.

In some embodiments, the subject has Type 1 diabetes. In some embodiments, the subject has Type 2 diabetes.

In some embodiments, the method described herein renders the subject insulin-independent.

In another aspect is provided a kit for preparing gastrin-treated islet cells, the kit comprising a gastrin composition and instructions for use.

In another aspect is provided a method of treating diabetes in a subject in need thereof, the method comprising administering a dosage of gastrin and a dosage of islet cells to the subject.

In some embodiments, the gastrin described herein comprises a polypeptide having an amino acid sequence having at least 70%, 75%, 80%, 85%, 90%, 95%, 96%, 97%, 98%, 99%, or more, identity to SEQ ID NO: 1 or 2. In some embodiments, the gastrin described herein comprises a polynucleotide encoding a polypeptide having an amino acid sequence having at least 70%, 75%, 80%, 85%, 90%, 95%, 96%, 97%, 98%, 99%, or more, identity to SEQ ID NO: 1 or 2.

In some embodiments, the islet cells are pre-treated with gastrin.

In some embodiments, the dosage of islet cells comprises less than 9,000 IEQ/kg, 8,000 IEQ/kg, 7,000 IEQ/kg, 6,000 IEQ/kg, 5,000 IEQ/kg, or less, of islet cells. In some embodiments, the dosage of islet cells comprises less than 9,000 IEQ/kg of islet cells.

In some embodiments, the gastrin is administered prior to, concurrently with, or after the administering of the dosage of islet cells.

In some embodiments, the gastrin is administered prior to the administering of the dosage of islet cells. In some embodiments, the gastrin is administered about one week, two weeks, three weeks, one month, or longer, prior to the administering of the dosage of islet cells. In some embodiments, the gastrin is administered continuously until at least one week, two weeks, three weeks, one month, two months, three months, four months, or longer, after the administering of the dosage of islet cell.

In some embodiments, the gastrin is administered concurrently with the administering of the dosage of islet cells.

In some embodiments, the gastrin is administered after the administering of the dosage of islet cells. In some embodiments, the gastrin is administered to the subject about one day, two days, three days, four days, five days, one week, two weeks, three weeks, one month, or longer, after the administration of the dosage of islet cells. In some embodiments, the gastrin is administered continuously until at least one week, two weeks, three weeks, one month, two months, three months, four months, or longer, after the administering of the dosage of islet cell.

In some embodiments, the gastrin is administered to the subject about two weeks prior to the administration of the dosage of islet cells, wherein the gastrin is continuously administered for two times per day, once per day, once per two days, once per three days, once per one week, or less frequent, for about one month, two months, three months, or longer. In some embodiments, the gastrin is administered to the subject about two weeks prior to the administration of the dosage of islet cells, wherein the gastrin is continuously administered until at least about one month after the administering of the dosage of islet cell.

In some embodiments, the gastrin is administered to the subject about two days after the administration of the dosage of islet cells, wherein the gastrin is continuously administered for two times per day, once per day, once per two days, once per three days, once per one week, or less frequent, for about one month, two months, three months, or longer. In some embodiments, the gastrin is administered to the subject about two days after the administration of the dosage of islet cells, wherein the gastrin is continuously administered until at least about one month after the administering of the dosage of islet cell.

In some embodiments, wherein the gastrin is administered to the subject once per day or two times per day.

In some embodiments, the gastrin is administered to the subject at a daily dosage of about 15 μg/kg to about 30 μg/kg, about 20 μg/kg to about 40 μg/kg, about 25 μg/kg to about 50 μg/kg, about 30 μg/kg to about 60 μg/kg, about 40 μg/kg to about 70 μg/kg, about 50 μg/kg to about 80 μg/kg, about 60 μg/kg to about 100 μg/kg, or more.

In some embodiments, the gastrin is administered to the subject subcutaneously, intramuscularly, intravenously, intrathecal, or any combination thereof. In some embodiments, the gastrin is administered to the subject subcutaneously.

In some embodiments, the method further comprises administering a second dosage of gastrin to the subject. In some embodiments, the second dosage of gastrin is administered to the subject about six months after administering the dosage of gastrin-treated human islet cells. In some embodiments, the second dosage of gastrin is administered to the subject is at least one time per day for about 30 days. In some embodiments, the second dosage of gastrin is administered to the subject two times per day. In some embodiments, the second dosage may comprise the same or different amounts of gastrin from the first dosage, or comprise the same or different dosing regimens (e.g., time periods for the whole dosing process or among individual dosages), which may be determined by a doctor or an authorized personnel.

In some embodiments, the method further comprises administering to the subject a proton pump inhibitor and a DPP-4 inhibitor. In some embodiments, the proton pump inhibitor is Esomeprazole. In some embodiments, the DPP-4 inhibitor is Sitagliptin.

In some embodiments, the subject has Type 1 diabetes. In some embodiments, the subject has Type 2 diabetes.

In some embodiments, the method described herein renders the subject insulin-independent.

In another aspect is provided a kit for preparing gastrin-treated islet cells, the kit including a gastrin composition and instructions for use.

### BRIEF DESCRIPTION OF THE DRAWINGS

FIG. 1. is a schematic showing the effect of gastrin on islet cells.

FIGS. 2A-2D. show Beta cell expansion/neogenesis in rats by LSC. FIG. 2A. Treatment groups and dose levels. FIG. 2B. Example rat islet image by LSC. FIG. 2C. Beta cells as percent of total cells per slide. FIG. 2D. Alpha cells as percent of total cells per slide.

FIG. 3. illustrates in vivo imaging of intraportally transplanted human islets in mouse liver with ¹⁸F-TCE4-PET with and without Gastrin-17 treatment.

FIG. 4. illustrates gastrin treatment promoted expansion of human islets following transplantation to murine livers.

FIG. 5. shows gastrin treatment promoted native pancreas islet expansion.

FIG. 6. illustrates beta cell mass is increased in livers of mice given Gastrin and human islets. Animals treated with Gastrin-17 had larger islet mass as reflected by higher % insulin staining area per tissue slide and larger number of slides with insulin+ cells.

FIG. 7 shows decreased blood glucose in mice treated with human islets+Gastrin-17 (Tx+ Treated) vs. islet transplant only (Tx only) vs. untreated control animals (Normal).

FIG. 8. shows that CCKBR is expressed in delta cells in healthy islets. Immunofluorescence staining for the CCKBR, insulin, glucagon, somatostatin and ductal marker CK19.

FIG. 9. illustrates Gastrin increases in insulin, somatostatin and glucagon mRNA in islets from donors with HbA1c >6.0%. qPCR analysis of RNA extracted from human islets treated with gastrin. Data are mean±SEM (n=5-6 donors in each group). * p<0.05, ** p<0.005.

FIG. 10. shows correlation between increase in insulin transcripts levels in response to gastrin and islet donor HbA1c levels. Correlation analysis between the increase in insulin transcripts in response to 48 hours 100 nM gastrin treatment on human islets and the HbA1c levels of each donor. (n=11, HbA1c 5.2-10.4).

FIG. 11. shows gastrin upregulates genes in islet beta and delta cell from donors with elevated HblAc on transcription factors. qPCR analysis of RNA extracted from human islets treated for 48 hours with increasing concentrations of gastrin. Data are mean±SEM (n=4-5 donors in each group). Increased transcription noted only in islets from the HbA1c>6.0% group. * p<0.05, ** p<0.005.

FIG. 12. illustrates that blocking gastrin receptor CCKBR inhibits gastrin induced increases in islet mRNA. qPCR analysis of RNA extracted from human islets treated for 48 hours with 100 nM gastrin with or without gastrin CCK receptor antagonist, YM022.

FIGS. 13A-13C. show that the gastrin analogue decreases long-term cultured human islet inflammation. Human islets from non-diabetic donors were cultured for ˜2 weeks±Gastrin 17 and mRNA levels assessed.

FIG. 14. shows blood glucose levels are lower in islet transplant recipients treated with PPI/DPP-4i.

FIG. 15. shows that gastrin reduced human islet damage from inflammatory cytokines, enhanced insulin secretion, and increased insulin+/somatostatin+ cell numbers.

FIGS. 16A-16C. illustrate that gastrin treatment in islet cell transplant (IT) provides insulin independence with a single procedure despite fewer islets given. Blood glucose (BG (mg/dl)), c-peptide (C-pep (ng/ml)), insulin intake (Insulin (U/dl)), and Hb A1c (A1c (%)) before and after IT in two T1D patients. FIG. 16A: IT without gastrin showing continuing need for insulin, deterioration of glycemic control after the first IT, and need for second IT; and, FIGS. 16B-16C: IT with gastrin (box) showing achieving insulin freedom and tight glycemic control with a single IT.

FIG. 17. illustrates that gastrin decreases islet cell death. Upper panel—control islets; lower panel—gastrin-treated (110 nM) islets. Dead cells stained with propidium iodide (red).

FIG. 18. illustrates that gastrin maintained islet function after long-term culture. Glucose challenge—upper graph; stimulation index—lower graph. Data represent mean SEM from a total three independent donors. ****p<0.0001 control versus gastrin.

FIG. 19. shows that gastrin suppresses expression of inflammatory genes in human islets. qRT-PCR analysis of proinflammatory related genes GCSF, GMCSF, IL-1b, IL-6, IL-10, TNFa, and CXCL1 in isolated human islets incubated±gastrin for 2 weeks. Data represent mean±SEM from a total of 20 independent donors with higher Hb A1c (n=5).

FIG. 20. illustrates that gastrin suppressed pro-inflammatory cytokine release from human islets after 2 weeks in culture. Luminex X-MAP assay measured IL-1β levels in the supernatant of gastrin (11 nM) treated and control islets. Data represent mean±SEM from a total of 11 independent donors of both high and lower Hb A1c. p<0.005.

FIG. 21. shows gastrin decreased apoptosis-related genes expression in isolated islets cultured for two weeks. qRT-PCR analysis selected genes. Data represent mean±SEM from a total of 25 independent donors of lower (black bars, n=17) and higher (grey bars, n=8) Hb A1c. 2-way ANOVA determined the significance.

FIG. 22. shows gastrin increased islet insulin+ cells in mice. NOD mice (age 8 weeks) received gastrin at several doses for 12 weeks. Islets were examined for insulin+ cell.

FIG. 23. shows that gastrin decreases insulites in diabetic mice. Tissue sections from diabetic mice. Islets from control animals show >50% inflammatory cell infiltration. Islets from 100 μg/kg gastrin treated showed less infiltration, while those from 600 μg/kg showed little infiltrate.

FIGS. 24A-24D. illustrate gastrin analogue GAST-17 stimulates beta cell expansion. Rats were treated with a gastrin analogue GAST-17 and pancreatic islet beta cell percentages determined. Group 1=controls.

FIG. 25. illustrates gastrin analogue GAST-17 promotes expansion/neogenesis of transplanted human islets. Isolated human islets were transplanted (Tx) to the livers of NOD mice followed by GAST-17 treatment. whole mice and organs of interest were imaged (in vivo and ex vivo) with 18F-TC-Exendin-4 (TCE4) using microPET.

FIG. 26. shows gastrin treatment in IT provides insulin independence with a single transplant despite fewer islets given. Blood glucose (green) and insulin intake (red) before and after IT in two T1D patients showing: deterioration of glycemic control after the first IT with no gastrin (upper panel); and achieving insulin freedom with a single IT with gastrin (light blue box) given at month 1 and 7 post-IT (lower panel).

FIG. 27. shows GLP-R1 localizes to native and transplanted islets. Immuno-fluorescent (IF) stained native human islets in the pancreas (A), and human islet grafts in mouse liver (B).

FIG. 28. shows radiosynthesis of 68Ga-DO3A-Exendin-4.

FIG. 29. shows radio-probe binds with affinity to GLP-1R expressing cells. Saturation binding analysis of [⁶⁸Ga]-DO3A-Exendin-4 in INS-1 cells (left graph). MicroPET images (right graph) of NOD/SCID mice bearing INS-1 cells without (left panel) and with (right panel) non-radiolabeled exendin-4.

FIG. 30. illustrates that the radiolabeled probe localizes to transplanted human islets. Coronal PET images of probe-treated mice 90 minutes post islet injection (left radiographs). Control mouse and mice with human islets. Kidneys were removed before microPET imaging. Quantification of liver uptake in the mice (right graph) (****p<0.001).

FIG. 31. illustrates the radio-probe distribution in pigs, non-human primates (“NHP”), and person.

FIG. 32. shows the Clinical Trial Study Design.

### DETAILED DESCRIPTION OF THE INVENTION

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those provided herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

### Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., ROR-1) in other proteins with different numbering systems. For example, by performing a simple sequence alignment with a protein (e.g., ROR-1) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “gastrin protein” or “gastrin” as used herein includes any of the recombinant or naturally-occurring forms of gastrin, or variants or homologs thereof that maintain gastrin activity (e.g. within at least 50%, 80%, 90%, 95%, 96%, 97%, 98%, 99% or 100% activity compared to gastrin). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gastrin protein. In embodiments, the gastrin protein is substantially identical to the protein identified by the UniProt reference number P01350 or a variant or homolog having substantial identity thereto. In embodiments, the term gastrin refers to a variant of gastrin. In embodiments, the term gastrin refers to a mature protein maintaining gastrin biological functions after cleavage of a gastrin precursor protein (e.g., a gastrin preproprotein). In embodiments, the gastrin preproprotein comprises an amino acid sequence shown in SEQ ID NO: 2 below (also in GenBank Access No.: NP_000796). In embodiments, the gastrin variant is gastrin-34, gastrin-17 or gastrin-14. In embodiments, the gastrin variant is gastrin-17. In embodiments, gastrin-17 includes the amino acid sequence Pyr-GPWLEEEEEAYGWMDF-NH2 (SEQ ID NO: 1). In embodiments, gastrin-17 is at least 80%, 85%, 90%, 95%, 96%, 97%, 98%, 99%, or more homologous or identical to the amino acid sequence of SEQ ID NO: 1. In embodiments, the gastrin variant is an analog of gastrin-17. In embodiments, the gastrin-17 analog is [Leu¹⁵] Gastrin-17 (GAST-17). In embodiments, the gastrin is or includes a human gastrin preproprotein amino acid sequence.

A human gastrin preproprotein amino acid sequence may be:

(SEQ ID NO: 2)
MQRLCVYVLIFALALAAFSEASWKPRSQQPDAPLGTGANRDLELPWLEQQ
GPASHHRRQLGPQGPPHLVADPSKKQGPWLEEEEEAYGWMDFGRRSAEDE
N

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

In embodiments, the term “gastrin” include any polypeptides (or any polynucleotides encoding such polypeptides) having at least 70%, 75%, 80%, 85%, 90%, 95%, 96%, 97%, 98%, 99% or more homologous or identical to the amino acid sequence of a polypeptide cleaved from a gastrin precursor protein (e.g., a human gastrin preproprotein having SEQ ID NO: 2), such as gastrin-17 having SEQ ID NO: 1.

“Contacting” is used in accordance with its plain ordinary meaning and refers to the process of allowing at least two distinct species (e.g. gastrin-17 and islet cells) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., Spodoptera) and human cells. In embodiments, the cell is an islet cell.

The term “pancreatic islets,” or “islets of Langerhans” as used herein refers to the regions of the pancreas that contain its endocrine (i.e., hormone-producing) cells. The pancreatic islets are arranged in density routes throughout the human pancreas, and are important in the metabolism of glucose. The term “islet cells” or “islets” as used herein refers to cells originated from a pancreatic islet. In embodiments, islet cells include alpha cells, beta cells, delta cells or a mixture thereof. In embodiments, islet cells include beta cells. Alpha cells (more commonly alpha-cells or α-cells) are endocrine cells in the pancreatic islets of the pancreas. They make up to about 20% of the human islet cells synthesizing and secreting the peptide hormone glucagon, which elevates the glucose levels in the blood. Beta cells make up about 50% to about 70% of islet cells. Beta cells synthesize and secrete insulin. Beta cells can respond quickly to spikes in blood glucose concentrations by secreting some of their stored insulin while simultaneously producing more. Delta cells (S-cells or D cells) are somatostatin-producing cells. They can be found in the stomach, intestine and the pancreatic islets.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “control” or “standard control” refers to a sample, measurement, or value that serves as a reference, usually a known reference, for comparison to a test sample, measurement, or value. For example, a test sample can be taken from a patient suspected of having a given disease (e.g. diabetes) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease (e.g. diabetes) or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be diabetes. The disease may be type I diabetes (T1D). The disease may be type II diabetes (T2D). Type 1 diabetes mellitus (T1D) precipitates from the autoimmune attack of pancreatic beta cells, resulting in a loss of functional beta cell mass. Thus, subjects with T1D do not make insulin or make very little insulin as compared to the standard amount produced by a subject without T1D. Functional beta cell mass is impacted positively by processes that increase the number and size of beta cells and negatively by those that deplete the numbers of cells (i.e., apoptosis, necrosis, and other modes of cell death). Type 2 diabetes (T2D) occurs when a subject is ineffective at using insulin that the body has produced (e.g. insulin resistance) and/or when a subject is unable to produce enough insulin. Thus, patients with T2D may have hyperglycemia (high blood glucose levels), due to lack of the standard effect of insulin (e.g. driving glucose in the blood inside the cells).

As used herein, the term “associated” or “associated with” in the context of a substance or substance activity or function associated with a disease (such as diabetes (T1D or T2D)) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, diabetes may be treated with a composition (e.g. gastrin-treated islet cells) effective for increasing beta cell production.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “allogeneic transplant” or “allogeneic transfusion” refers to the transfer of biological material (e.g. islet cells) to a recipient from a genetically non-identical donor of the same species. The transplant may be referred to as an allograft, allogeneic transplant, or homograft. For example, a tissue or organ transplant may be an allogeneic transplant. An allogeneic transplant may include transfer of tissue, a group of cells or an organ to a recipient that is genetically non-identical to the donor. For example, the transplant may be a bone marrow transplant comprising islet cells from the donor.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

### Methods

The methods provided herein including embodiments thereof are contemplated to be effective for treating diabetes (e.g. type I diabetes, type II diabetes) in a subject in need thereof. The methods include treating the subject with a dosage of gastrin-treated human islet cells. In embodiments, the dosage is a single dosage. As used herein, “single dosage” refers to not administering a second dosage or subsequent dosage of gastrin-treated human islet cells to the subject for a pre-determined amount of time after administration of the dosage of gastrin-treated human islet cells. In embodiments, the second dosage is not administered to the subject for at least 1 week, 2 weeks, 3 weeks, 1 month, 1.5 months, 2 months, 2.5 months, 3 months, 3.5 months, 4 months, 4.5 months, 5 months, 5.5 months, 6 months, 7 months, 8 months, 9 months, 10 months, 11 months, or a year after administration of the dosage of gastrin-treated human islet cells. Thus, in embodiments, a second dosage of gastrin-treated human islet cells is not administered to the subject for at least 1 week after administering the dosage of gastrin-treated human islet cells. In embodiments, a second dosage of gastrin-treated human islet cells is not administered to the subject for at least 1 month after administering the dosage of gastrin-treated human islet cells. In embodiments, a second dosage of gastrin-treated human islet cells is not administered to the subject for at least 1 year after administering the dosage of gastrin-treated human islet cells.

In embodiments, administration of the dosage of gastrin-treated human islet cells results in the subject being insulin independent (e.g. not requiring administration of exogenous insulin). In embodiments, administration of the dosage (e.g. single dosage) of gastrin-treated human islet cells reduces risks associated with administration of multiple dosages of gastrin-treated human islet cells. For example, the risks associated with administration of multiple dosages include transplant rejection due to multiple antigen loads and infections. In embodiments, a single dosage administration reduces the requirement for administration of anti-rejection drugs to the subject, and additionally is more cost-effective and a more convenient treatment method compared to treatment methods including multi-dosage administration of human islet cells.

Thus, in an aspect is provided a method of treating diabetes in a subject in need thereof, the method including administering a dosage of gastrin-treated human islet cells to the subject, wherein the dosage includes less than 9,000 IEQ/kg of islet cells. In embodiments, the dosage includes less than 8,000 IEQ/kg of islet cells. In embodiments, the dosage includes less than 7,000 IEQ/kg of islet cells. In embodiments, the dosage includes less than 6,000 IEQ/kg of islet cells. In embodiments, the dosage includes less than 5,000 IEQ/kg of islet cells.

As used herein, “gastrin-treated” refers to a cell, compound, composition, etc. that has been contacted with gastrin or an analog or derivative thereof. For example, a gastrin-treated islet cell refers to an islet cell that has been contacted with gastrin (e.g. cultured in a suitable media in the presence of gastrin). For example, an islet cell from a human donor (e.g. a subject without diabetes) may be cultured in standard islet medium in the present of gastrin, thereby resulting in gastrin-treated islet cells.

In embodiments, the islet cell is cultured in media including from about 10 nM to about 250 nM gastrin. In embodiments, the islet cell is cultured in media including from about 20 nM to about 250 nM gastrin. In embodiments, the islet cell is cultured in media including from about 30 nM to about 250 nM gastrin. In embodiments, the islet cell is cultured in media including from about 40 nM to about 250 nM gastrin. In embodiments, the islet cell is cultured in media including from about 50 nM to about 250 nM gastrin. In embodiments, the islet cell is cultured in media including from about 60 nM to about 250 nM gastrin. In embodiments, the islet cell is cultured in media including from about 70 nM to about 250 nM gastrin. In embodiments, the islet cell is cultured in media including from about 80 nM to about 250 nM gastrin. In embodiments, the islet cell is cultured in media including from about 90 nM to about 250 nM gastrin. In embodiments, the islet cell is cultured in media including from about 100 nM to about 250 nM gastrin. In embodiments, the islet cell is cultured in media including from about 110 nM to about 250 nM gastrin. In embodiments, the islet cell is cultured in media including from about 120 nM to about 250 nM gastrin. In embodiments, the islet cell is cultured in media including from about 130 nM to about 250 nM gastrin. In embodiments, the islet cell is cultured in media including from about 140 nM to about 250 nM gastrin. In embodiments, the islet cell is cultured in media including from about 150 nM to about 250 nM gastrin. In embodiments, the islet cell is cultured in media including from about 160 nM to about 250 nM gastrin. In embodiments, the islet cell is cultured in media including from about 170 nM to about 250 nM gastrin. In embodiments, the islet cell is cultured in media including from about 180 nM to about 250 nM gastrin. In embodiments, the islet cell is cultured in media including from about 190 nM to about 250 nM gastrin. In embodiments, the islet cell is cultured in media including from about 200 nM to about 250 nM gastrin. In embodiments, the islet cell is cultured in media including from about 210 nM to about 250 nM gastrin. In embodiments, the islet cell is cultured in media including from about 220 nM to about 250 nM gastrin. In embodiments, the islet cell is cultured in media including from about 230 nM to about 250 nM gastrin. In embodiments, the islet cell is cultured in media including from about 240 nM to about 250 nM gastrin.

In embodiments, the islet cell is cultured in media including from about 10 nM to about 240 nM gastrin. In embodiments, the islet cell is cultured in media including from about 10 nM to about 230 nM gastrin. In embodiments, the islet cell is cultured in media including from about 10 nM to about 220 nM gastrin. In embodiments, the islet cell is cultured in media including from about 10 nM to about 210 nM gastrin. In embodiments, the islet cell is cultured in media including from about 10 nM to about 200 nM gastrin. In embodiments, the islet cell is cultured in media including from about 10 nM to about 190 nM gastrin. In embodiments, the islet cell is cultured in media including from about 10 nM to about 180 nM gastrin. In embodiments, the islet cell is cultured in media including from about 10 nM to about 170 nM gastrin. In embodiments, the islet cell is cultured in media including from about 10 nM to about 160 nM gastrin. In embodiments, the islet cell is cultured in media including from about 10 nM to about 150 nM gastrin. In embodiments, the islet cell is cultured in media including from about 10 nM to about 140 nM gastrin. In embodiments, the islet cell is cultured in media including from about 10 nM to about 130 nM gastrin. In embodiments, the islet cell is cultured in media including from about 10 nM to about 120 nM gastrin. In embodiments, the islet cell is cultured in media including from about 10 nM to about 110 nM gastrin. In embodiments, the islet cell is cultured in media including from about 10 nM to about 100 nM gastrin. In embodiments, the islet cell is cultured in media including from about 10 nM to about 90 nM gastrin. In embodiments, the islet cell is cultured in media including from about 10 nM to about 80 nM gastrin. In embodiments, the islet cell is cultured in media including from about 10 nM to about 70 nM gastrin. In embodiments, the islet cell is cultured in media including from about 10 nM to about 60 nM gastrin. In embodiments, the islet cell is cultured in media including from about 10 nM to about 40 nM gastrin. In embodiments, the islet cell is cultured in media including from about 10 nM to about 30 nM gastrin. In embodiments, the islet cell is cultured in media including from about 10 nM to about 20 nM gastrin. In embodiments, the islet cell is cultured in media including from about 10 nM, 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM or 250 nM. In embodiments, the islet cell is cultured in media including about 100 nM gastrin.

In embodiments, the islet cells are cultured for about 2 days to about 30 days. In embodiments, the islet cells are cultured for about 4 days to about 30 days. In embodiments, the islet cells are cultured for about 6 days to about 30 days. In embodiments, the islet cells are cultured for about 8 days to about 30 days. In embodiments, the islet cells are cultured for about 10 days to about 30 days. In embodiments, the islet cells are cultured for about 12 days to about 30 days. In embodiments, the islet cells are cultured for about 14 days to about 30 days. In embodiments, the islet cells are cultured for about 16 days to about 30 days. In embodiments, the islet cells are cultured for about 18 days to about 30 days. In embodiments, the islet cells are cultured for about 20 days to about 30 days. In embodiments, the islet cells are cultured for about 22 days to about 30 days. In embodiments, the islet cells are cultured for about 24 days to about 30 days. In embodiments, the islet cells are cultured for about 26 days to about 30 days. In embodiments, the islet cells are cultured for about 28 days to about 30 days.

In embodiments, the islet cells are cultured for about 2 days to about 28 days. In embodiments, the islet cells are cultured for about 2 days to about 26 days. In embodiments, the islet cells are cultured for about 2 days to about 24 days. In embodiments, the islet cells are cultured for about 2 days to about 22 days. In embodiments, the islet cells are cultured for about 2 days to about 20 days. In embodiments, the islet cells are cultured for about 2 days to about 18 days. In embodiments, the islet cells are cultured for about 2 days to about 16 days. In embodiments, the islet cells are cultured for about 2 days to about 14 days. In embodiments, the islet cells are cultured for about 2 days to about 12 days. In embodiments, the islet cells are cultured for about 2 days to about 10 days. In embodiments, the islet cells are cultured for about 2 days to about 8 days. In embodiments, the islet cells are cultured for about 2 days to about 6 days. In embodiments, the islet cells are cultured for about 2 days to about 4 days. In embodiments, the islet cells are cultured for about 2 days, 4 days, 6 days, 8 days, 10 days, 12 days, 14 days, 16 days, 18 days, 20 days, 22 days, 24 days, 26 days, 28 days, or 30. In embodiments, the islet cells are cultured for about 14 days.

As used herein, “IEQ” refers to islet equivalent numbers wherein an islet equivalent is equal to the volume of an islet with 150 μm diameter.

In embodiments, the dosage is about 5,000 IEQ/kg of islet cells to about 9,000 IEQ/kg of islet cells. In embodiments, the dosage is about 5,250 IEQ/kg of islet cells to about 9,000 IEQ/kg of islet cells. In embodiments, the dosage is about 5,500 IEQ/kg of islet cells to about 9,000 IEQ/kg of islet cells. In embodiments, the dosage is about 5,750 IEQ/kg of islet cells to about 9,000 IEQ/kg of islet cells. In embodiments, the dosage is about 6,000 IEQ/kg of islet cells to about 9,000 IEQ/kg of islet cells. In embodiments, the dosage is about 6,250 IEQ/kg of islet cells to about 9,000 IEQ/kg of islet cells. In embodiments, the dosage is about 6,500 IEQ/kg of islet cells to about 9,000 IEQ/kg of islet cells. In embodiments, the dosage is about 6,750 IEQ/kg of islet cells to about 9,000 IEQ/kg of islet cells. In embodiments, the dosage is about 7,000 IEQ/kg of islet cells to about 9,000 IEQ/kg of islet cells. In embodiments, the dosage is about 7,250 IEQ/kg of islet cells to about 9,000 IEQ/kg of islet cells. In embodiments, the dosage is about 7,500 IEQ/kg of islet cells to about 9,000 IEQ/kg of islet cells. In embodiments, the dosage is about 7,750 IEQ/kg of islet cells to about 9,000 IEQ/kg of islet cells. In embodiments, the dosage is about 8,000 IEQ/kg of islet cells to about 9,000 IEQ/kg of islet cells. In embodiments, the dosage is about 8,250 IEQ/kg of islet cells to about 9,000 IEQ/kg of islet cells. In embodiments, the dosage is about 8,500 IEQ/kg of islet cells to about 9,000 IEQ/kg of islet cells. In embodiments, the dosage is about 8,750 IEQ/kg of islet cells to about 9,000 IEQ/kg of islet cells. In embodiments, the dosage is about 6,000 IEQ/kg of islet cells to about 9,000 IEQ/kg of islet cells.

In embodiments, the dosage is about 5,000 IEQ/kg of islet cells to about 8,750 IEQ/kg of islet cells. In embodiments, the dosage is about 5,000 IEQ/kg of islet cells to about 8,500 IEQ/kg of islet cells. In embodiments, the dosage is about 5,000 IEQ/kg of islet cells to about 8,250 IEQ/kg of islet cells. In embodiments, the dosage is about 5,000 IEQ/kg of islet cells to about 7,000 IEQ/kg of islet cells. In embodiments, the dosage is about 5,000 IEQ/kg of islet cells to about 6,750 IEQ/kg of islet cells. In embodiments, the dosage is about 5,000 IEQ/kg of islet cells to about 6,500 IEQ/kg of islet cells. In embodiments, the dosage is about 5,000 IEQ/kg of islet cells to about 6,250 IEQ/kg of islet cells. In embodiments, the dosage is about 5,000 IEQ/kg of islet cells to about 6,000 IEQ/kg of islet cells. In embodiments, the dosage is about 5,000 IEQ/kg of islet cells to about 5,750 IEQ/kg of islet cells. In embodiments, the dosage is about 5,000 IEQ/kg of islet cells to about 5,500 IEQ/kg of islet cells. In embodiments, the dosage is about 5,000 IEQ/kg of islet cells to about 5,250 IEQ/kg of islet cells. In embodiments, the dosage is about 5,000 IEQ/kg of islet cells, 5,250 IEQ/kg of islet cells, 5,500 IEQ/kg of islet cells, 5,750 IEQ/kg of islet cells, 6,000 IEQ/kg of islet cells, 6,250 IEQ/kg of islet cells, 6,500 IEQ/kg of islet cells, 6,750 IEQ/kg of islet cells, 7,000 IEQ/kg of islet cells, 7,250 IEQ/kg of islet cells, 7,500 IEQ/kg of islet cells, 7,750 IEQ/kg of islet cells, 8,000 IEQ/kg of islet cells, 8,250 IEQ/kg of islet cells, 8,500 IEQ/kg of islet cells, 8,750 IEQ/kg of islet cells or 9,000 IEQ/kg of islet cells.

In embodiments, the dosage is about 250 IEQ/kg of islet cells to about 5,000 IEQ/kg of islet cells. In embodiments, the dosage is about 500 IEQ/kg of islet cells to about 5,000 IEQ/kg of islet cells. In embodiments, the dosage is about 750 IEQ/kg of islet cells to about 5,000 IEQ/kg of islet cells. In embodiments, the dosage is about 1000 IEQ/kg of islet cells to about 5,000 IEQ/kg of islet cells. In embodiments, the dosage is about 1,250 IEQ/kg of islet cells to about 5,000 IEQ/kg of islet cells. In embodiments, the dosage is about 1,500 IEQ/kg of islet cells to about 5,000 IEQ/kg of islet cells. In embodiments, the dosage is about 1,750 IEQ/kg of islet cells to about 5,000 IEQ/kg of islet cells. In embodiments, the dosage is about 2,000 IEQ/kg of islet cells to about 5,000 IEQ/kg of islet cells. In embodiments, the dosage is about 2,250 IEQ/kg of islet cells to about 5,000 IEQ/kg of islet cells. In embodiments, the dosage is about 2,500 IEQ/kg of islet cells to about 5,000 IEQ/kg of islet cells. In embodiments, the dosage is about 2,750 IEQ/kg of islet cells to about 5,000 IEQ/kg of islet cells. In embodiments, the dosage is about 3,000 IEQ/kg of islet cells to about 5,000 IEQ/kg of islet cells. In embodiments, the dosage is about 3,250 IEQ/kg of islet cells to about 5,000 IEQ/kg of islet cells. In embodiments, the dosage is about 3,500 IEQ/kg of islet cells to about 5,000 IEQ/kg of islet cells. In embodiments, the dosage is about 3,750 IEQ/kg of islet cells to about 5,000 IEQ/kg of islet cells. In embodiments, the dosage is about 4,000 IEQ/kg of islet cells to about 5,000 IEQ/kg of islet cells. In embodiments, the dosage is about 4,250 IEQ/kg of islet cells to about 5,000 IEQ/kg of islet cells. In embodiments, the dosage is about 4,500 IEQ/kg of islet cells to about 5,000 IEQ/kg of islet cells. In embodiments, the dosage is about 4,750 IEQ/kg of islet cells to about 5,000 IEQ/kg of islet cells.

In embodiments, the dosage is about 250 IEQ/kg of islet cells to about 4,750 IEQ/kg of islet cells. In embodiments, the dosage is about 250 IEQ/kg of islet cells to about 4,500 IEQ/kg of islet cells. In embodiments, the dosage is about 250 IEQ/kg of islet cells to about 4,250 IEQ/kg of islet cells. In embodiments, the dosage is about 250 IEQ/kg of islet cells to about 4,000 IEQ/kg of islet cells. In embodiments, the dosage is about 250 IEQ/kg of islet cells to about 3,750 IEQ/kg of islet cells. In embodiments, the dosage is about 250 IEQ/kg of islet cells to about 3,500 IEQ/kg of islet cells. In embodiments, the dosage is about 250 IEQ/kg of islet cells to about 3,250 IEQ/kg of islet cells. In embodiments, the dosage is about 250 IEQ/kg of islet cells to about 3,000 IEQ/kg of islet cells. In embodiments, the dosage is about 250 IEQ/kg of islet cells to about 2,750 IEQ/kg of islet cells. In embodiments, the dosage is about 250 IEQ/kg of islet cells to about 2,500 IEQ/kg of islet cells. In embodiments, the dosage is about 250 IEQ/kg of islet cells to about 2,250 IEQ/kg of islet cells. In embodiments, the dosage is about 250 IEQ/kg of islet cells to about 2,000 IEQ/kg of islet cells. In embodiments, the dosage is about 250 IEQ/kg of islet cells to about 1,750 IEQ/kg of islet cells. In embodiments, the dosage is about 250 IEQ/kg of islet cells to about 1,500 IEQ/kg of islet cells. In embodiments, the dosage is about 250 IEQ/kg of islet cells to about 1,250 IEQ/kg of islet cells. In embodiments, the dosage is about 250 IEQ/kg of islet cells to about 1,000 IEQ/kg of islet cells. In embodiments, the dosage is about 250 IEQ/kg of islet cells to about 750 IEQ/kg of islet cells. In embodiments, the dosage is about 250 IEQ/kg of islet cells to about 500 IEQ/kg of islet cells. In embodiments, the dosage is about 250 IEQ/kg of islet cells, 500 IEQ/kg of islet cells, 750 IEQ/kg of islet cells, 1000 IEQ/kg of islet cells, 1,250 IEQ/kg of islet cells, 1,500 IEQ/kg of islet cells, 1,750 IEQ/kg of islet cells, 2,000 IEQ/kg of islet cells, 2,250 IEQ/kg of islet cells, 2,500 IEQ/kg of islet cells, 2,750 IEQ/kg of islet cells, 3,000 IEQ/kg of islet cells, 3,250 IEQ/kg of islet cells, 3,500 IEQ/kg of islet cells, 3,750 IEQ/kg of islet cells, 4,000 IEQ/kg of islet cells, 4,250 IEQ/kg of islet cells, 4,500 IEQ/kg of islet cells, 4,750 IEQ/kg of islet cells, or 5,000 IEQ/kg of islet cells.

In embodiments, the gastrin-treated human islet cells are treated with gastrin or an analog or derivative thereof. In embodiments, the gastrin-treated human islet cells are treated with gastrin-17 or an analog or derivative thereof. In embodiments, the gastrin-treated human islet cells are treated with gastrin-17. As used herein, “gastrin-17”, also known as little gastrin I, refers to a cleavage product of gastrin. In embodiments, the gastrin-treated human islet cells are treated with a gastrin-17 analog (e.g. [Leu¹⁵] Gastrin-17 (GAST-17)). Compositions including gastrin, which may be used to treat the gastrin-treated cells provided herein including embodiments thereof, are described in detail in US 20110034379 and US201000256061, which are incorporated herein in their entirety and for all purposes.

In embodiments, the human islet cells are not obtained from the subject. Thus, in embodiments, the human islet cells are allogenic human islet cells. As used herein, “allogenic human islet cells” refers to islet cells that are transferred to the recipient from a genetically non-identical donor of the same species.

For the methods provided herein, in embodiments, the gastrin-treated human islet cells are obtained by a method including: (a) culturing islet cells from a donor; (b) contacting the culture with gastrin; and, harvesting the islet cells. In embodiments, the culture is contacted with gastrin or an analog or derivative thereof. In embodiments, the method further includes administering to the subject gastrin. In embodiments, the gastrin includes gastrin-17 or a derivative or analog thereof. In embodiments, the gastrin is gastrin-17 or a derivative or analog thereof. In embodiments, the gastrin includes GAST-17. In embodiments, the gastrin is GAST-17. For example, the gastrin may be provided as a GAST-17 lyophilized powder, wherein the powder is reconstituted in a distilled water or a suitable buffer prior to administration. In embodiments, the gastrin is provided as a 1 mg, 1.5 mg, 2 mg, 2.5 mg, 5 mg, 7.5 mg or 10 mg lyophilized powder of gastrin-17 or a derivative or analog thereof (e.g. GAST-17), wherein the powder is reconstituted by a suitable volume of distilled water or buffer prior to administration. In embodiments, the gastrin is administered by injection.

In embodiments, the gastrin is administered to the subject prior to administration of the dosage of the gastrin-treated human islet cells. In embodiments, the gastrin is administered to the subject after the administration of the dosage of gastrin-treated human islet cells. In embodiments, the gastrin is administered to the subject at the same time (concurrently) to administration of the dosage of the gastrin-treated human islet cells. In embodiments, the gastrin is administered to the subject about one day prior to the administration of the dosage of gastrin-treated human islet cells. In embodiments, the gastrin is administered to the subject about two days prior to the administration of the dosage of gastrin-treated human islet cells. In embodiments, the gastrin is administered to the subject about three days prior to the administration of the dosage of gastrin-treated human islet cells. In embodiments, the gastrin is administered to the subject about four days prior to the administration of the dosage of gastrin-treated human islet cells. In embodiments, the gastrin is administered to the subject about five days prior to the administration of the dosage of gastrin-treated human islet cells. n embodiments, the gastrin is administered to the subject about six days prior to the administration of the dosage of gastrin-treated human islet cells. In embodiments, the gastrin is administered to the subject about one week prior to the administration of the dosage of gastrin-treated human islet cells. In embodiments, the gastrin is administered to the subject about ten days prior to the administration of the dosage of gastrin-treated human islet cells. In embodiments, the gastrin is administered to the subject about two weeks prior to the administration of the dosage of gastrin-treated human islet cells. In embodiments, the gastrin is administered to the subject about three weeks prior to the administration of the dosage of gastrin-treated human islet cells. In embodiments, the gastrin is administered to the subject about one month prior to the administration of the dosage of gastrin-treated human islet cells. In embodiments, the gastrin is administered to the subject longer than about one month prior to the administration of the dosage of gastrin-treated human islet cells. In embodiments, the gastrin is administered to the subject about one day after the administration of the dosage of gastrin-treated human islet cells. In embodiments, the gastrin is administered to the subject about two days after the administration of the dosage of gastrin-treated human islet cells. In embodiments, the gastrin is administered to the subject about three days after the administration of the dosage of gastrin-treated human islet cells. In embodiments, the gastrin is administered to the subject about four days after the administration of the dosage of gastrin-treated human islet cells. In embodiments, the gastrin is administered to the subject about five days after the administration of the dosage of gastrin-treated human islet cells. In embodiments, the gastrin is administered to the subject about six days after the administration of the dosage of gastrin-treated human islet cells. In embodiments, the gastrin is administered to the subject about one week after the administration of the dosage of gastrin-treated human islet cells. In embodiments, the gastrin is administered to the subject about ten days after the administration of the dosage of gastrin-treated human islet cells. In embodiments, the gastrin is administered to the subject about two weeks after the administration of the dosage of gastrin-treated human islet cells. In embodiments, the gastrin is administered to the subject about three weeks after the administration of the dosage of gastrin-treated human islet cells. In embodiments, the gastrin is administered to the subject about one month after the administration of the dosage of gastrin-treated human islet cells. In embodiments, the gastrin is administered to the subject about two months after the administration of the dosage of gastrin-treated human islet cells. In embodiments, the gastrin is administered to the subject about three months after the administration of the dosage of gastrin-treated human islet cells. In embodiments, the gastrin is administered to the subject longer than about three months after the administration of the dosage of gastrin-treated human islet cells. In embodiments, the gastrin is administered to the subject at least one time per day. In embodiments, the gastrin is administered to the subject two times per day. In embodiments, the gastrin is administered to the subject three times per day. In embodiments, the gastrin is administered to the subject four times per day. In embodiments, the gastrin is administered to the subject at least one time per day for at least about 30 days. In embodiments, the gastrin is administered to the subject at least two times per day for about 30 days. In embodiments, the gastrin is administered to the subject about one day, two days, three days, four days, five days, six days, one week, ten days, two weeks, three weeks, one month, or longer, prior to the administration of the dosage of gastrin-treated human islet cells, and the gastrin is continuously administered until at least about one day, two days, three days, four days, five days, six days, one week, ten days, two weeks, three weeks, one month, two months, three months, four months, five months, six months, or later, after the administration of the dosage of gastrin-treated human islet cells. In embodiments, the gastrin is administered to the subject at least about one day prior to the administration of the dosage of gastrin-treated human islet cells, while the gastrin is continuously administered until at least about three days after the administration of the dosage of gastrin-treated human islet cells. In embodiments, the gastrin is administered to the subject at least about one week prior to the administration of the dosage of gastrin-treated human islet cells, while the gastrin is continuously administered until at least about one week after the administration of the dosage of gastrin-treated human islet cells. In embodiments, the gastrin is administered to the subject at least about two weeks prior to the administration of the dosage of gastrin-treated human islet cells, while the gastrin is continuously administered until at least about one month after the administration of the dosage of gastrin-treated human islet cells. In embodiments, the gastrin is administered to the subject at least about two weeks prior to the administration of the dosage of gastrin-treated human islet cells, while the gastrin is continuously administered until at least about two months after the administration of the dosage of gastrin-treated human islet cells. In embodiments, the gastrin is administered to the subject at least about two weeks prior to the administration of the dosage of gastrin-treated human islet cells, while the gastrin is continuously administered until at least about three months after the administration of the dosage of gastrin-treated human islet cells. In embodiments, the gastrin is administered to the subject at least about two weeks prior to the administration of the dosage of gastrin-treated human islet cells, while the gastrin is continuously administered until at least about four months after the administration of the dosage of gastrin-treated human islet cells. In embodiments, the gastrin is administered to the subject at least about one month prior to the administration of the dosage of gastrin-treated human islet cells, while the gastrin is continuously administered until at least about one month after the administration of the dosage of gastrin-treated human islet cells. In embodiments, the gastrin is administered to the subject at least about one month prior to the administration of the dosage of gastrin-treated human islet cells, while the gastrin is continuously administered until at least about two months after the administration of the dosage of gastrin-treated human islet cells. In embodiments, the gastrin is administered to the subject at least about one month prior to the administration of the dosage of gastrin-treated human islet cells, while the gastrin is continuously administered until at least about three months after the administration of the dosage of gastrin-treated human islet cells. In embodiments, the gastrin is administered to the subject at least about one month prior to the administration of the dosage of gastrin-treated human islet cells, while the gastrin is continuously administered until at least about four months after the administration of the dosage of gastrin-treated human islet cells. In embodiments, the gastrin is continuously administered to the subject at least once per day, two times per day, three times per day, four times per day, once per two days, once per three days, once per four days, once per five days, once per one week, once per two weeks, or less frequently.

In embodiments, the gastrin is administered to the subject about two days before the administration of the dosage of gastrin-treated human islet cells for two times per day. In embodiments, the gastrin is administered to the subject about two days before the administration of the dosage of gastrin-treated human islet cells for three times per day. In embodiments, the gastrin is administered to the subject about two days before the administration of the dosage of gastrin-treated human islet cells for four times per day.

In embodiments, the gastrin is administered to the subject about two days after the administration of the dosage of gastrin-treated human islet cells for two times per day for about 30 days. In embodiments, GAST-17 is administered to the subject about two days after the administration of the dosage of gastrin-treated human islet cells for two times per day for about 30 days, wherein the administration of GAST-17 is subcutaneous, and wherein the dosage of GAST-17 is about 15 μg/kg.

For the methods provided herein, in embodiments, the gastrin is administered to the subject at a dosage of about 10 μg/kg to about 20 μg/kg. In embodiments, the gastrin is administered to the subject at a dosage of about 10.5 μg/kg to about 20 μg/kg. In embodiments, the gastrin is administered to the subject at a dosage of about 11 μg/kg to about 20 μg/kg. In embodiments, the gastrin is administered to the subject at a dosage of about 11.5 μg/kg to about 20 μg/kg. In embodiments, the gastrin is administered to the subject at a dosage of about 12 μg/kg to about 20 μg/kg. In embodiments, the gastrin is administered to the subject at a dosage of about 12.5 μg/kg to about 20 μg/kg. In embodiments, the gastrin is administered to the subject at a dosage of about 13 μg/kg to about 20 μg/kg. In embodiments, the gastrin is administered to the subject at a dosage of about 13.5 μg/kg to about 20 μg/kg. In embodiments, the gastrin is administered to the subject at a dosage of about 14 μg/kg to about 20 μg/kg. In embodiments, the gastrin is administered to the subject at a dosage of about 14.5 μg/kg to about 20 μg/kg. In embodiments, the gastrin is administered to the subject at a dosage of about 15 μg/kg to about 20 μg/kg. In embodiments, the gastrin is administered to the subject at a dosage of about 15.5 μg/kg to about 20 μg/kg. In embodiments, the gastrin is administered to the subject at a dosage of about 16 μg/kg to about 20 μg/kg. In embodiments, the gastrin is administered to the subject at a dosage of about 16.5 μg/kg to about 20 μg/kg. In embodiments, the gastrin is administered to the subject at a dosage of about 17 μg/kg to about 20 μg/kg. In embodiments, the gastrin is administered to the subject at a dosage of about 17.5 μg/kg to about 20 μg/kg. In embodiments, the gastrin is administered to the subject at a dosage of about 18 μg/kg to about 20 μg/kg. In embodiments, the gastrin is administered to the subject at a dosage of about 18.5 μg/kg to about 20 μg/kg. In embodiments, the gastrin is administered to the subject at a dosage of about 19 μg/kg to about 20 μg/kg. In embodiments, the gastrin is administered to the subject at a dosage of about 19.5 μg/kg to about 20 μg/kg.

In embodiments, the gastrin is administered to the subject at a dosage of about 10 μg/kg to about 19.5 μg/kg. In embodiments, the gastrin is administered to the subject at a dosage of about 10 μg/kg to about 19 μg/kg. In embodiments, the gastrin is administered to the subject at a dosage of about 10 μg/kg to about 18.5 μg/kg. In embodiments, the gastrin is administered to the subject at a dosage of about 10 μg/kg to about 18 μg/kg. In embodiments, the gastrin is administered to the subject at a dosage of about 10 μg/kg to about 17.5 μg/kg. In embodiments, the gastrin is administered to the subject at a dosage of about 10 μg/kg to about 17 μg/kg. In embodiments, the gastrin is administered to the subject at a dosage of about 10 μg/kg to about 16.5 μg/kg. In embodiments, the gastrin is administered to the subject at a dosage of about 10 μg/kg to about 16 μg/kg. In embodiments, the gastrin is administered to the subject at a dosage of about 10 μg/kg to about 15.5 μg/kg. In embodiments, the gastrin is administered to the subject at a dosage of about 10 μg/kg to about 15 μg/kg. In embodiments, the gastrin is administered to the subject at a dosage of about 10 μg/kg to about 14.5 μg/kg. In embodiments, the gastrin is administered to the subject at a dosage of about 10 μg/kg to about 14 μg/kg. In embodiments, the gastrin is administered to the subject at a dosage of about 10 μg/kg to about 13.5 μg/kg. In embodiments, the gastrin is administered to the subject at a dosage of about 10 μg/kg to about 13 μg/kg. In embodiments, the gastrin is administered to the subject at a dosage of about 10 μg/kg to about 12.5 μg/kg. In embodiments, the gastrin is administered to the subject at a dosage of about 10 μg/kg to about 12 μg/kg. In embodiments, the gastrin is administered to the subject at a dosage of about 10 μg/kg to about 11.5 μg/kg. In embodiments, the gastrin is administered to the subject at a dosage of about 10 μg/kg to about 11 μg/kg. In embodiments, the gastrin is administered to the subject at a dosage of about 10 μg/kg to about 10.5 μg/kg. In embodiments, the gastrin is administered to the subject at a dosage of about 10 μg/kg, 10.5 μg/kg, 11 μg/kg, 11.5 μg/kg, 12 μg/kg, 12.5 μg/kg, 13 μg/kg, 13.5 μg/kg, 14 μg/kg, 14.5 μg/kg, 15 μg/kg, 15.5 μg/kg, 16 μg/kg, 16.5 μg/kg, 17 μg/kg, 17.5 μg/kg, 18 μg/kg, 18.5 μg/kg, 19 μg/kg, 19.5 μg/kg or 20 μg/kg. In embodiments, the gastrin is administered to the subject at a dosage of about 15 μg/kg. In embodiments, the gastrin is administered to the subject subcutaneously.

In embodiments, the gastrin is administered to the subject at a dosage of about 10 μg/kg to about 35 μg/kg. In embodiments, the gastrin is administered to the subject at a dosage of about 11 μg/kg to about 35 μg/kg. In embodiments, the gastrin is administered to the subject at a dosage of about 12 μg/kg to about 35 μg/kg. In embodiments, the gastrin is administered to the subject at a dosage of about 13 μg/kg to about 35 μg/kg. In embodiments, the gastrin is administered to the subject at a dosage of about 14 μg/kg to about 35 μg/kg. In embodiments, the gastrin is administered to the subject at a dosage of about 15 μg/kg to about 35 μg/kg. In embodiments, the gastrin is administered to the subject at a dosage of about 16 μg/kg to about 35 μg/kg. In embodiments, the gastrin is administered to the subject at a dosage of about 17 μg/kg to about 35 μg/kg. In embodiments, the gastrin is administered to the subject at a dosage of about 18 μg/kg to about 35 μg/kg. In embodiments, the gastrin is administered to the subject at a dosage of about 19 μg/kg to about 35 μg/kg. In embodiments, the gastrin is administered to the subject at a dosage of about 20 μg/kg to about 35 μg/kg. In embodiments, the gastrin is administered to the subject at a dosage of about 21 μg/kg to about 35 μg/kg. In embodiments, the gastrin is administered to the subject at a dosage of about 22 μg/kg to about 35 μg/kg. In embodiments, the gastrin is administered to the subject at a dosage of about 23 μg/kg to about 35 μg/kg. In embodiments, the gastrin is administered to the subject at a dosage of about 24 μg/kg to about 35 μg/kg. In embodiments, the gastrin is administered to the subject at a dosage of about 25 μg/kg to about 35 μg/kg. In embodiments, the gastrin is administered to the subject at a dosage of about 26 μg/kg to about 35 μg/kg. In embodiments, the gastrin is administered to the subject at a dosage of about 27 μg/kg to about 35 μg/kg. In embodiments, the gastrin is administered to the subject at a dosage of about 28 μg/kg to about 35 μg/kg. In embodiments, the gastrin is administered to the subject at a dosage of about 29 μg/kg to about 35 μg/kg. In embodiments, the gastrin is administered to the subject at a dosage of about 30 μg/kg to about 35 μg/kg. In embodiments, the gastrin is administered to the subject at a dosage of about 31 μg/kg to about 35 μg/kg. In embodiments, the gastrin is administered to the subject at a dosage of about 32 μg/kg to about 35 μg/kg. In embodiments, the gastrin is administered to the subject at a dosage of about 33 μg/kg to about 35 μg/kg. In embodiments, the gastrin is administered to the subject at a dosage of about 34 μg/kg to about 35 μg/kg.

In embodiments, the gastrin is administered to the subject at a dosage of about 10 μg/kg to about 34 μg/kg. In embodiments, the gastrin is administered to the subject at a dosage of about 10 μg/kg to about 33 μg/kg. In embodiments, the gastrin is administered to the subject at a dosage of about 10 μg/kg to about 32 μg/kg. In embodiments, the gastrin is administered to the subject at a dosage of about 10 μg/kg to about 31 μg/kg. In embodiments, the gastrin is administered to the subject at a dosage of about 10 μg/kg to about 30 μg/kg. In embodiments, the gastrin is administered to the subject at a dosage of about 10 μg/kg to about 29 μg/kg. In embodiments, the gastrin is administered to the subject at a dosage of about 10 μg/kg to about 28 μg/kg. In embodiments, the gastrin is administered to the subject at a dosage of about 10 μg/kg to about 27 μg/kg. In embodiments, the gastrin is administered to the subject at a dosage of about 10 μg/kg to about 26 μg/kg. In embodiments, the gastrin is administered to the subject at a dosage of about 10 μg/kg to about 25 μg/kg. In embodiments, the gastrin is administered to the subject at a dosage of about 10 μg/kg to about 24 μg/kg. In embodiments, the gastrin is administered to the subject at a dosage of about 10 μg/kg to about 23 μg/kg. In embodiments, the gastrin is administered to the subject at a dosage of about 10 μg/kg to about 22 μg/kg. In embodiments, the gastrin is administered to the subject at a dosage of about 10 μg/kg to about 21 μg/kg. In embodiments, the gastrin is administered to the subject at a dosage of about 10 μg/kg to about 20 μg/kg. In embodiments, the gastrin is administered to the subject at a dosage of about 10 μg/kg to about 19 μg/kg. In embodiments, the gastrin is administered to the subject at a dosage of about 10 μg/kg to about 18 μg/kg. In embodiments, the gastrin is administered to the subject at a dosage of about 10 μg/kg to about 17 μg/kg. In embodiments, the gastrin is administered to the subject at a dosage of about 10 μg/kg to about 16 μg/kg. In embodiments, the gastrin is administered to the subject at a dosage of about 10 μg/kg to about 15 μg/kg. In embodiments, the gastrin is administered to the subject at a dosage of about 10 μg/kg to about 14 μg/kg. In embodiments, the gastrin is administered to the subject at a dosage of about 10 μg/kg to about 13 μg/kg. In embodiments, the gastrin is administered to the subject at a dosage of about 10 μg/kg to about 12 μg/kg. In embodiments, the gastrin is administered to the subject at a dosage of about 10 μg/kg to about 11 μg/kg. In embodiments, the gastrin is administered to the subject at a dosage of about 10 μg/kg to about 10 μg/kg. In embodiments, the gastrin is administered to the subject at a dosage of about 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg or 30 μg/kg.

In embodiments, the gastrin is administered to the subject at a dosage of about 10 μg/kg to about 60 μg/kg per day (daily). In embodiments, the gastrin is administered to the subject at a dosage of about 10 μg/kg to about 70 μg/kg per day (daily). In embodiments, the gastrin is administered to the subject at a dosage of about 10 μg/kg to about 80 μg/kg per day (daily). In embodiments, the gastrin is administered to the subject at a dosage of about 10 μg/kg to about 90 μg/kg per day (daily). In embodiments, the gastrin is administered to the subject at a dosage of about 10 μg/kg to about 100 μg/kg per day (daily). In embodiments, the gastrin is administered to the subject at a dosage of about 20 μg/kg to about 60 μg/kg per day (daily). In embodiments, the gastrin is administered to the subject at a dosage of about 20 μg/kg to about 70 μg/kg per day (daily). In embodiments, the gastrin is administered to the subject at a dosage of about 20 μg/kg to about 80 μg/kg per day (daily). In embodiments, the gastrin is administered to the subject at a dosage of about 20 μg/kg to about 90 μg/kg per day (daily). In embodiments, the gastrin is administered to the subject at a dosage of about 20 μg/kg to about 100 μg/kg per day (daily). In embodiments, the gastrin is administered to the subject at a dosage of about 30 μg/kg to about 60 μg/kg per day (daily). In embodiments, the gastrin is administered to the subject at a dosage of about 30 μg/kg to about 70 μg/kg per day (daily). In embodiments, the gastrin is administered to the subject at a dosage of about 30 μg/kg to about 80 μg/kg per day (daily). In embodiments, the gastrin is administered to the subject at a dosage of about 30 μg/kg to about 90 μg/kg per day (daily). In embodiments, the gastrin is administered to the subject at a dosage of about 30 μg/kg to about 100 μg/kg per day (daily). In embodiments, the gastrin is administered to the subject at a dosage of about 40 μg/kg to about 60 μg/kg per day (daily). In embodiments, the gastrin is administered to the subject at a dosage of about 40 μg/kg to about 70 μg/kg per day (daily). In embodiments, the gastrin is administered to the subject at a dosage of about 40 μg/kg to about 80 μg/kg per day (daily). In embodiments, the gastrin is administered to the subject at a dosage of about 40 μg/kg to about 90 μg/kg per day (daily). In embodiments, the gastrin is administered to the subject at a dosage of about 40 μg/kg to about 100 μg/kg per day (daily). Such dosage, as a total daily dosage, may be administered for one time, two times, three times, four times, or more frequent, per day. For example, if administered one time per day, the gastrin may be administered to the subject at a daily dosage of about 10 μg/kg to about 60 μg/kg, about 10 μg/kg to about 70 μg/kg, about 10 μg/kg to about 80 μg/kg, about 10 μg/kg to about 90 μg/kg, about 10 μg/kg to about 100 μg/kg, about 20 μg/kg to about 60 μg/kg, about 20 μg/kg to about 70 μg/kg, about 20 μg/kg to about 80 μg/kg, about 20 μg/kg to about 90 μg/kg, about 20 μg/kg to about 100 μg/kg, about 30 μg/kg to about 60 μg/kg, about 30 μg/kg to about 70 μg/kg, about 30 μg/kg to about 80 μg/kg, about 30 μg/kg to about 90 μg/kg, about 30 μg/kg to about 100 μg/kg, about 40 μg/kg to about 60 μg/kg, about 40 μg/kg to about 70 μg/kg, about 40 μg/kg to about 80 μg/kg, about 40 μg/kg to about 90 μg/kg, or about 40 μg/kg to about 100 μg/kg, while if administered two times per day, in each time the gastrin may be administered to the subject at a dosage of about 5 μg/kg to about 30 μg/kg, about 5 μg/kg to about 35 μg/kg, about 5 μg/kg to about 40 μg/kg, about 5 μg/kg to about 45 μg/kg, about 5 μg/kg to about 50 μg/kg, about 10 μg/kg to about 30 μg/kg, about 10 μg/kg to about 35 μg/kg, about 10 μg/kg to about 40 μg/kg, about 10 μg/kg to about 45 μg/kg, about 10 μg/kg to about 50 μg/kg, about 15 μg/kg to about 30 μg/kg, about 15 μg/kg to about 35 μg/kg, about 15 μg/kg to about 40 μg/kg, about 15 μg/kg to about 45 μg/kg, about 15 μg/kg to about 50 μg/kg, about 20 μg/kg to about 30 μg/kg, about 20 μg/kg to about 35 μg/kg, about 20 μg/kg to about 40 μg/kg, about 20 μg/kg to about 45 μg/kg, or about 20 μg/kg to about 50 μg/kg.

In embodiments, the method further includes administering a second dosage of gastrin to the subject. In embodiments, the second dosage of gastrin is administered to the subject about three months after administering the dosage of gastrin-treated human islet cells. In embodiments, the second dosage of gastrin is administered to the subject about four months after administering the dosage of gastrin-treated human islet cells. In embodiments, the second dosage of gastrin is administered to the subject about five months after administering the dosage of gastrin-treated human islet cells. In embodiments, the second dosage of gastrin is administered to the subject about six months after administering the dosage of gastrin-treated human islet cells. In embodiments, the second dosage of gastrin is administered to the subject about seven months after administering the dosage of gastrin-treated human islet cells. In embodiments, the second dosage of gastrin is administered to the subject about eight months after administering the dosage of gastrin-treated human islet cells. In embodiments, the second dosage of gastrin is administered to the subject about nine months after administering the dosage of gastrin-treated human islet cells. In embodiments, the second dosage of gastrin is administered to the subject about 10 months after administering the dosage of gastrin-treated human islet cells. In embodiments, the second dosage of gastrin is administered to the subject about 11 months after administering the dosage of gastrin-treated human islet cells. In embodiments, the second dosage of gastrin is administered to the subject about 12 months after administering the dosage of gastrin-treated human islet cells. In embodiments, the second dosage of gastrin is administered to the subject is at least one time per day. In embodiments, the second dosage of gastrin is administered to the subject is at least one time per day for about 30 days. In embodiments, the second dosage of gastrin is administered to the subject two times per day. In embodiments, the second dosage of gastrin is administered to the subject is at least two times per day for about 30 days.

It is contemplated that administration of a proton pump inhibitor (PPI) (e.g. Esomeprazole (Nexium)) is effective for reducing side effects associated with gastrin induced gastric acid secretion. It is further contemplated that PPI may augment endogenous gastric secretion. Further, Applicant has found that administration of a DPP-4 inhibitor (e.g. Sitagliptin (Januvia)) may enhance the half-life of endogenous GLP-1 half-life, thereby increasing the biological effect on insulin secretion.

As used herein, the terms “proton pump inhibitor” or “PPI” refer to a class of compounds that reduce or down-regulate the production of stomach acid. Typically, a PPI functions by inhibiting the hydrogen/potassium adenosine triphosphatase (H⁺/K⁺ ATPase) enzyme system in the stomach. Protein pump inhibitors include Omeprazole, Lansoprazole, Dexlansoprazole, Esomeprazole, Pantoprazole, Rabeprazole and Ilaprazole.

As used herein, the terms “dipeptidyl peptidase 4 inhibitor” or “DPP-4 inhibitor”, also known as gliptins, refer to a class of compounds that block or down-regulate the activity of the enzyme dipeptidyl peptidase-4 (DPP-4). DPP-4 inhibitors may be used to lower glucose for treatment of type 2 diabetes. Typically, DPP-4 inhibitors inhibit DPP-4 activity in peripheral plasma, thereby preventing inactivation of glucagon-like peptide (GLP)-1 in the peripheral circulation. This may increase circulating GLP-1, resulting in increased insulin secretion and decreased glucagon secretion, thus increasing glucose utilization and diminishing hepatic glucose reduction. Through this mechanism, HbA1c may be reduced. DPP-4 inhibitors include Sitagliptin, Vildagliptin, Saxagliptin, Linagliptin, Gemigliptin, Anagliptin, Teneligliptin, Alogliptin, Trelagliptin, Omarigliptin, Evogliptin, Gosogliptin and Dutogliptin.

Thus, in embodiments, the method further includes administering to the subject a proton pump inhibitor and a DPP-4 inhibitor. In embodiments, the method further includes administering to the subject a proton pump inhibitor or a DPP-4 inhibitor. In embodiments, the method further includes administering to the subject a proton pump inhibitor. In embodiments, the method further includes administering to the subject a DPP-4 inhibitor. In embodiments, the proton pump inhibitor is Esomeprazole. In embodiments, the DPP-4 inhibitor is Sitagliptin.

In embodiments, the PPI is administered two times daily at a dosage of 40 mg. In embodiments, the PPI is administered orally. In embodiments, the DPP-4 is administered two times daily at a dosage of 50 mg twice daily. In embodiments, the DPP-4 is administered orally.

In embodiments, the subject has Type 1 diabetes. In embodiments, the subject has Type 2 diabetes. For the methods provided herein, in embodiments, the subject is rendered insulin-independent (e.g. does not require administration of exogenous insulin).

In an aspect is provided a kit for preparing gastrin-treated islet cells including a gastrin composition and instructions for use. In embodiments, the kit includes infusion media. In embodiments, the kit includes a container for the islets.

### EXAMPLES

### Example 1: Background to Exemplary Studies

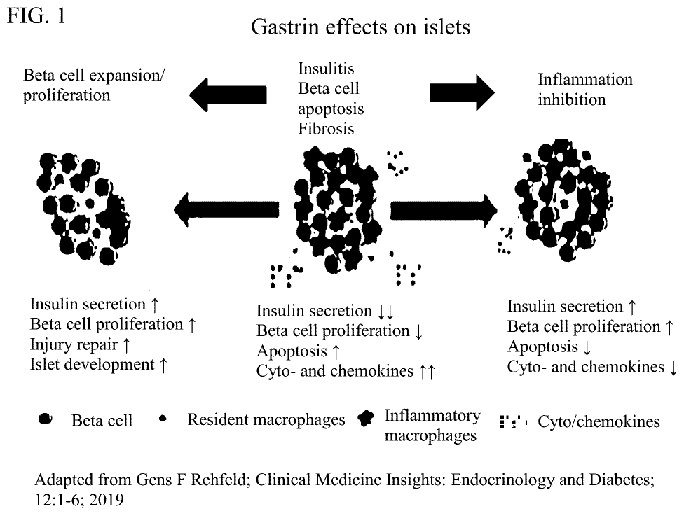

Gastrin is a hormone secreted from fetal pancreatic G cells to regulate beta cell development and from adult stomach G cells to regulate acid secretion. Gastrin is expressed in the insulin+ and somatostatin+ islet cells of people with T2D. It was shown that gastrin promotes beta cell proliferation and possibly differentiation of pancreatic ductal cells into insulin+ cells. It was found that human islets from elevated HbA1c donors treated with gastrin showed increased expression of islet hormones (insulin, glucagon, somatostatin) and beta cell transcription factors (PDX1, MNX1, SMAD9, HHEX, MAFA, SOX5). Also, gastrin stimulated the transformation of delta cells into insulin+/somatostatin+ cells, with increased insulin gene expression correlating positively with donor HbA1c levels. Data also showed that long-term islet exposure to gastrin increased expression of NGN3, nestin, urocortin3, PPY, and MAFB, and increased cell proliferation and numbers of insulin+/somatostatin+ cells, while reducing inflammatory gene expression. Gastrin additionally protected islets from inflammatory cytokines and increased their insulin production in response to glucose stimulation. Thus, gastrin is a promising islet hormone secretagogue, an inhibitor of islet inflammation, and a promotor of cell growth/trans-differentiation. Moreover, the beneficial effects are most evident in individuals with elevated HbA1c who have more beta cell dysfunction (FIG. 1).

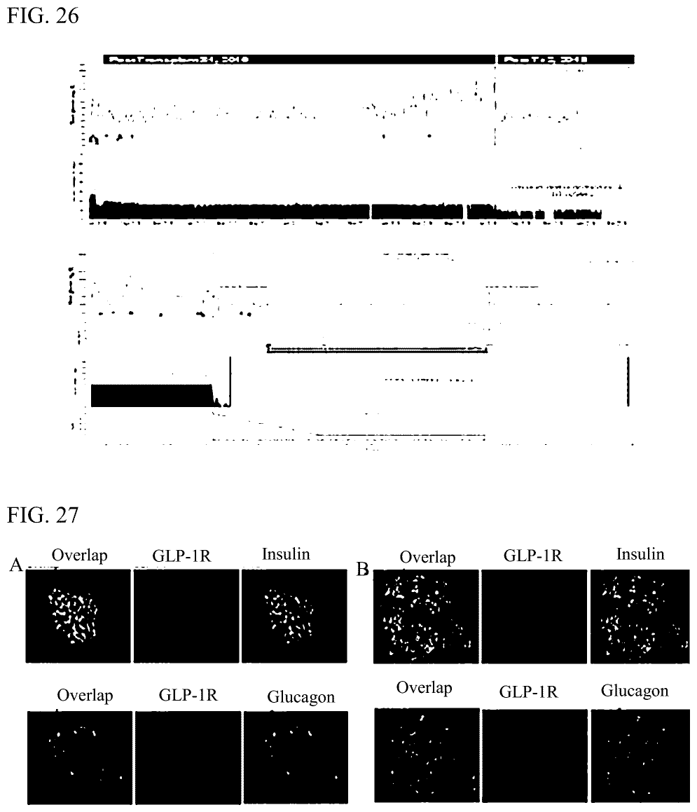

A clinical grade gastrin analogue (GAST-17) was manufactured with FDA approval for an ongoing clinical trial evaluating its use to improve islet function in type 1 diabetic islet transplant recipients. Initial results are promising, with the first two individuals treated with GAST-17 and a single isle transplant achieving insulin independence with half of the islet mass normally required (FIG. 26). These data show that GAST-17 promotes beta cell differentiation/neogenesis, and insulin secretion, while reducing islet and systemic inflammation to improve insulin secretion and sensitivity in individuals with type 1 diabetes (T1D) and type 2 Diabetes (T2D).

A wide array of therapeutic agents for T1D and T2D are available but none simultaneously target islet inflammation and beta cell expansion/neogenesis. Most drugs ignore the ongoing inflammation and diminished islet beta cell mass. Even GLP-1, another gut hormone, and its analogues, do not expand beta cells at clinically approved doses.

### Example 2: Improvement of Islet Engraftment, Induction of Beta Cell Expansion/Function and Enhancement of Islet Cell Transplant Outcomes by Gastrin Treatment

Gastrin is a natural hormone that is secreted by the stomach and is involved in fetal pancreas development. Preclinical and clinical data suggest that gastrin can act as an insulin secretagogue, promote beta cell proliferation/transdifferentiation, and also inhibit inflammation, making it an excellent candidate to address unresolved challenges in IT. Building on these observations, it was tested whether gastrin treatment will improve islet engraftment, induced beta cell expansion/function, and enhanced islet transplant outcomes in T1D individuals. A gastrin analogue (GAST-17) was produced and obtained FDA-IND approval to evaluate its safety and efficacy in a Phase I/II, prospective, single-arm trial to improve outcomes in T1D recipients undergoing a single islet transplant and two 30-day courses for GAST-17. The trial has been initiated with the first two treated individuals achieving insulin independence with roughly half of the islet dose normally required.

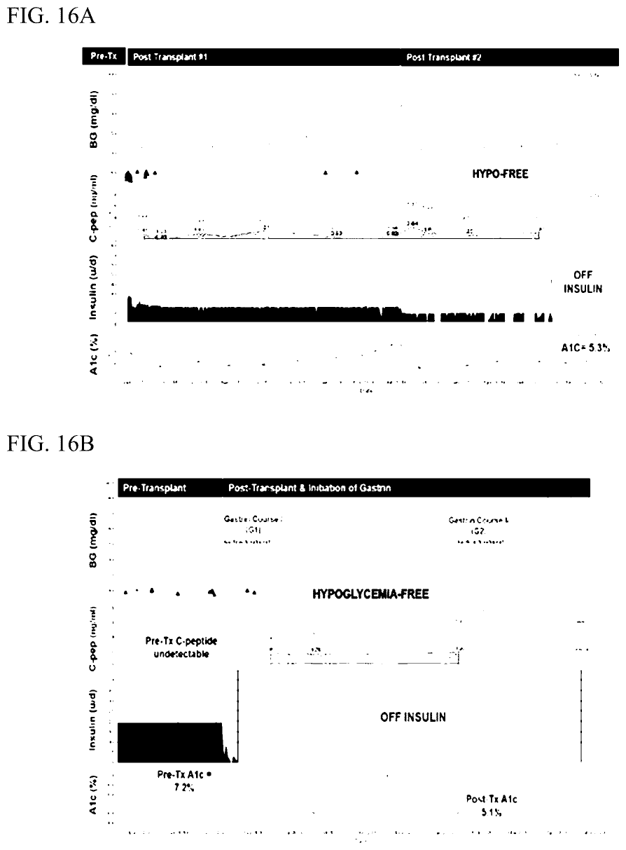

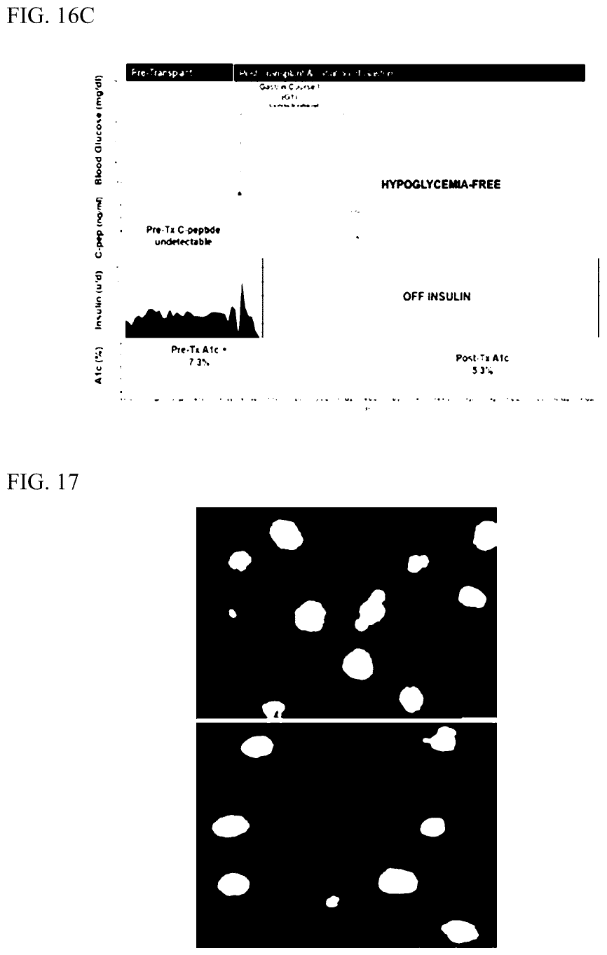

The success of clinical islet transplantation depends on transplantation of adequate islet mass and minimizing graft loss secondary to ischemia and inflammation. After infusion into the portal vein, islets are exposed to damaging inflammatory factors (the so-called instant blood-mediated inflammatory reaction, IBMIR). This involves activation of the complement and coagulation cascades, ultimately resulting in clot formation and infiltration of leukocytes into the islets. The IBMIR may be triggered by islet surface molecules, such as tissue factor and collagen residues that are normally not in direct contact with the blood. Also, ischemic stress during islet isolation results in production of inflammatory mediators by islets. Strategies to mitigate the IBMIR include the treatment of recipients with anticoagulants or pre-conditioning islets with anti-inflammatory agents. As inflammatory reactions are thought to negatively impact islet engraftment in the early post-transplant period, etanercept, a TNFα mitigator, and anakinra, an IL-1 receptor antagonist, are used to limit acute transplant-related inflammation. Preliminary data indicate that etanercept and anakinra, along with T-cell depletion, are safe, well-tolerated and associated with early insulin independence with normal HbA1c levels [32, 33]. Despite the above strategies, most IT recipients require transplantation of >9,000 islet equivalents (IEQ)/kg BW in order to achieve insulin sufficiency (15) and therefore, require multiple islet infusions. In contrast, two patients treated with gastrin analogue, [Leu¹⁵] Gastrin-17 (GAST-17), and a single IT (<6,100 IEQ/kg BW) achieved insulin freedom within two weeks (FIG. 16), possibly due to added protection by anti-inflammatory properties of gastrin.

Lack of adequate immunosuppressive coverage at time of islet transplant may also decrease graft survival. Commencing the immunosuppression immediately prior to the first islet infusion, may not allow time to achieve targeted drug levels. This lack of adequate immunosuppressive may cause graft loss. This is consistent with the observations in patients who had sub-optimal immune suppression with sirolimus and tacrolimus and had minimal reduction in insulin requirements, suggesting poor islet survival. Using a more potent, T lymphocyte depleting induction regimen with recombinant ATG in one small study was associated with insulin independence in all single islet transplant recipients (13), suggesting excellent early graft survival and engraftment. However, all subjects have subsequently returned to insulin intake. In a larger cumulative series collected by the Collaborative Islet Transplant Registry (CITR), 50-60% of islet recipients who received immunosuppressive induction including T-cell depletion were insulin free at 5-years compared to 15-20% not receiving T-cell depletion (16, 17). While these results represent significant improvement in initial islet survival, a decline in islet graft function over time is still evident.

Mechanisms of islet graft functional decline over time.

The mechanisms underlying loss of islet graft function over time are multifactorial. One potential cause of graft failure is immunologic rejection, either acute or chronic. Reactivation of the autoimmune response in T1D individuals is a threat to the survival of transplanted islets. It was believed that immunosuppression was able to block activation of allo- and auto-immune responses (18-20). However, advances in detecting autoreactive T cells suggested that recurrence of islet-autoimmunity occurs. Indeed, significant correlation between cellular autoreactivity, as measured by lymphocyte stimulation tests against autoantigens, and clinical islet transplant outcomes was reported (21).

Drug-induced islet toxicity may also play a role in islet graft dysfunction. Tacrolimus has side effects even at low doses (22, 23). In experience, elevated levels of sirolimus were associated with islet graft dysfunction. Thus, drug-related toxicities may be responsible for the late islet graft “exhaustion” and failure. However, high levels of sirolimus and tacrolimus are necessary to avoid islet injury from alloreactivity. Thus, protection of the islet graft requires drug levels that, in themselves, compromise islet graft function.

Another possible cause of islet graft dysfunction is islet exhaustion due to inadequate islet mass. The innate human pancreas contains approximately 1 million islets (24). However, only about half of the islets are procured with current islet isolation methods. Also, less than 50% of transplanted islets engraft (25-27). Thus, as little as 15% of the normal pancreas islet mass remains functional after islet transplantation (13, 28). This low islet mass, together with chronic exposure to high glucose and toxins in the liver, leads to gradual decline in transplanted islet function. The trend of gradual loss of islet function over time has been demonstrated by all leading transplant groups (29-31), The fact that insulin independence rates decline, while C-peptide secretion persists for years thereafter (29-31), gives credence to the theory that the islet graft is functionally compromised over time (islet exhaustion). Thus, availability of therapies that can expand beta cell number and/or function after IT may result in long-term insulin freedom.

Role of Gastrin to improve IT outcomes

A potential strategy for achieving insulin independence with a smaller islet mass is by introducing factors known to stimulate islet cell neogenesis. There has been a great deal of research interest focused on the use of incretin and other potential beta cell growth factors to expand beta cell mass. Primary among these are gastrin, clustrin, epidermal growth factor (EGF) and glucagon like peptide-1 (GLP-1) (32-35).

Gastrin is a peptide that exists in the G-cells of the pancreas during fetal development. After birth, it disappears from the pancreas, but it continues to be produced by the G-cells of the stomach to regulate acid secretion. Experimental studies showed gastrin can induce beta cell neogenesis from pancreatic exocrine duct cell in rodents (36, 37) and increases homeobox transcription factor PDX-1, a critical factor in beta cell neogenesis (38). Gastrin may also promote beta cell proliferation and neogenesis indirectly through increasing the production of clustrin. Recent data (below) suggests gastrin stimulates pancreatic delta cells to express both insulin and somatostatin, raising the possibility that delta cells may constitute an alternative progenitor cell source within the islets. Combined treatment with gastrin and epidermal growth factor (EGF) (39) ameliorated hyperglycemia in diabetic mice.

There have been limited clinical trials examining these factors for the treatment of diabetes. Transitional Therapeutics, Inc. conducted Phase I and II clinical trials by of gastrin analogue with and without EGF analogue in patients with type 1 and type 2 diabetes and showed a favorable safety profile and a reduction in daily insulin requirements (40). The most common adverse events observed were nausea and headache. Twelve weeks after cessation of gastrin/EGF treatment, the patients' insulin requirements were reduced by ˜40%. This suggests that gastrin treatment may have increased beta cell mass, as the effects persisted after cessation of treatment. Fifty-four percent of T1D subjects responded to gastrin/EGF treatment either with a reduction of average daily intake by >20% or reduction in HbA1c (62).

Proton pump inhibitors (PPI) increase gastrin concentrations (41). Studies showed a positive effect of PPIs on glycemic control in diabetics, presumably through gastrin-stimulation of insulin secretion (42-44). Interestingly, treatment with a PPI reduced HbA1c in T2D individuals with poor glycemic control (45). It was reported that low-dose gastrin and EGF induced ductal cell trans-differentiation into beta cells in mice with moderate hyperglycemia (46). Further, it was found that human islets from donors with an HbA1c >6% demonstrated more robust increases in insulin gene expression in response to gastrin than islets from donors with a normal HbA1c. It was also shown that gastrin expression is reactivated in the islets of diabetic rodents and people with T2D (47).

The REPAIR-T1D trial examined the effects sitagliptin and lansoprazole in patients with recent onset T1D (48). The expected increases in gastrin blood levels were not observed, and there was no significant difference in C-peptide. The lack of response may be due to failure to achieve adequate elevation in serum gastrin levels to induce beta cell expansion. In the absence of immunosuppression therapy, it is also possible that the rate of autoimmune beta cell destruction exceeded the rate of cell neogenesis. Still, PPIs improve glycemic control as observed in islet transplant recipients.

### Significance

This clinical islet transplantation trial utilizes T-cell depleting immunosuppressive induction, double anti-inflammatory blockage peri-transplant with etanercept and anakinra, 3-drug maintenance immunosuppression with tacrolimus, MMF and sirolimus, and islet graft support with the gastrin analogue (GAST-17), oral PPI and DPP-4i. Gastrin+PPI+DPP-4i treatment is expected to induce beta cell expansion/neogenesis and enhance beta cell functional capacity. The data provided herein indicates that GAST-17 may also reduce inflammation within the islet cell microenvironment, which could improve islet engraftment and survival. These combined effects allow for greater improvement in glycemic control and possible insulin independence with islet transplant from a single donor. The trial also seeks to identify factors predictive of islet outcomes and help improve the understanding of mechanisms underlying islet graft dysfunction and rejection. Formal analysis of quality of life (QOL) changes serves to characterize the benefits of islet transplantation stimulation therapy. Findings from this trial has further reaching benefits for T1D management beyond the setting of islet transplant. For example, the regimen can be potentially applied to expand residual beta cell mass in new onset T1D. Finally, identification of new biomarkers predictive of islet/beta cell loss can allow for earlier diagnosis of T1D and/or earlier detection of islet graft loss.

### Islet Preparation and Transplantation.

Islets are prepared using methods approved by the FDA (BB-MF 9986, BB-IND 9988). COH initiated its first islet transplant trial testing the safety and efficacy of islet transplantation alone (ITA) in patients with T1D complicated by hypoglycemia in April 2004. A total of 17 subjects were treated each receiving up to 4 islet infusions in order to achieve an islet mass of >9,000 IEQ/kg BW. Twelve subjects completed their treatment course (Table 1). Results from multiple sources indicate that islet transplantation is able to reduce/eliminate insulin requirements and hypoglycemia and improve overall blood glucose control, in some individuals for over 10 years. Interim results from the ongoing clinical trial exploring the benefit of T-cell depleting immunosuppression induction on IT safety and efficacy, 8 of 8 subjects (100%) who have been followed through at least Day 75 post-IT achieved blood glucose stabilization (HbA1c <7% and no severe hypoglycemia) and 5 of 8 subjects (63%) transplant achieved insulin independence. Four out of 5 of these subjects required >9,000 IEQ/kg BW; the 1 subject who achieved insulin independence with a single transplant had low body mass (52 kg) and low daily insulin requirements (16 u/day) and represents the only transplant recipient to discontinue insulin treatment after a single transplant outside the gastrin-treated subjects.

TABLE 1
Efficacy Summary among ITA Subjects Who Completed Study Treatment (n = 12).
Data used for assessment of efficacy was collected
within 30 days of the study time point date.
Number and Percentage of Evaluable islet transplant alone protocol
(ITA) Subjects Meeting the Study Efficacy Criteria

| | Time (months) Post Last Transplant (all had >1 transplant) | | | |
|---|---|---|---|---|
| | 3 mo | 6 mo | 12 mo | 24 mo |
| Efficacy Parameter | (n = 12) | (n = 12) | (n = 12) | (n = 11) |
| Subjects off insulin | 11/12 | 11/12 | 9/12 | 8/11 |
| | (92%) | (92%) | (75%) | (73%) |
| Stimulated C-peptide >0.6 ng/ml | 12/12 | 12/12 | 11/12 | 10/11 |
| | (100%) | (100%) | (92%) | (91%) |
| Insulin requirements <0.2 | 12/12 | 12/12 | 11/12 | 9/11 |
| units/kg/day | (100%) | (100%) | (92%) | (82%) |
| Hypoglycemia free* | 11/12 | 12/12 | 9/12 | 10/11 |
| | (92%) | (100%) | (75%) | (91%) |
| HbA1c 6.5% | 11/12 | 10/12 | 10/12 | 6/11** |
| | (92%) | (83%) | (83%) | (55%) |

*Hypoglycemia free is defined as no blood glucoses <60 mg/dl during the specified month.

Gastrin increases beta cell mass in rats.

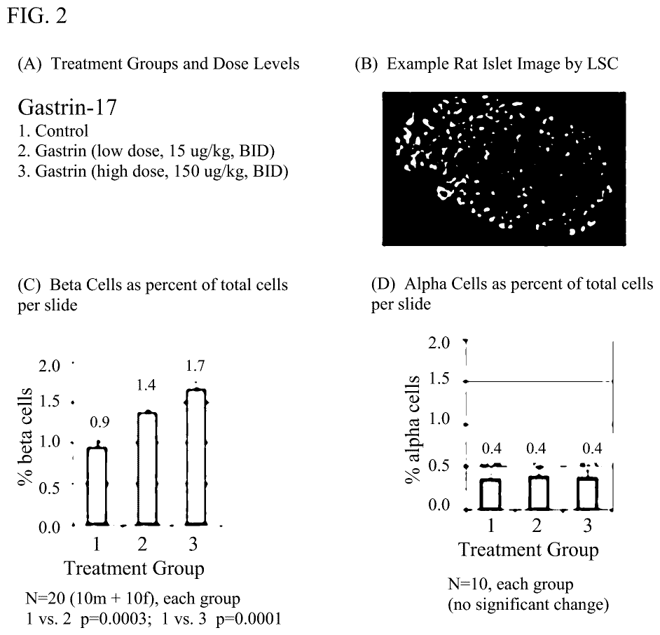

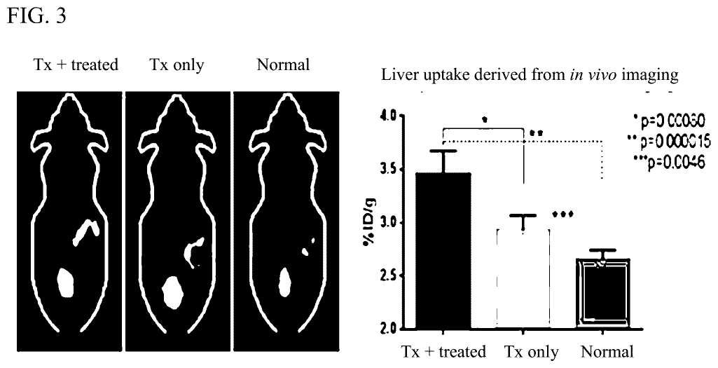

The islet expansion effects of GAST-17 were evaluated in non-diabetic Wistar rats (10 males and 10 females in each group). At the end of 30-day treatment, animals were terminated, pancreata excised and immuno-stained for beta and alpha cell content using laser scanning cytometry (LSC) (FIGS. 2A and 2B). The average percentage of beta cells significantly increased in all gastrin treatment groups compared to controls, while the percentage of alpha cells did not change (FIGS. 2C and 2D).

Gastrin promotes expansion/neogenesis of transplanted human islets.

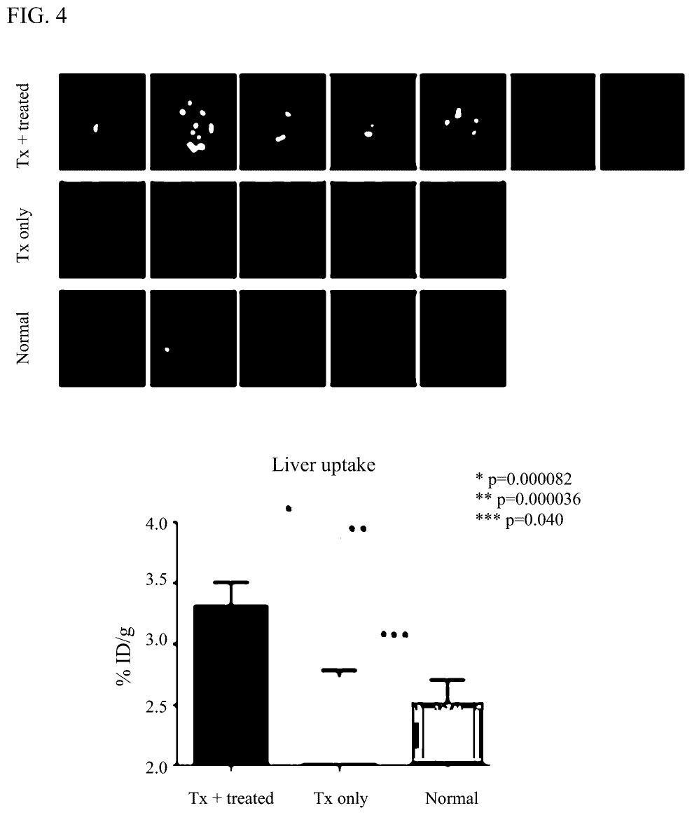

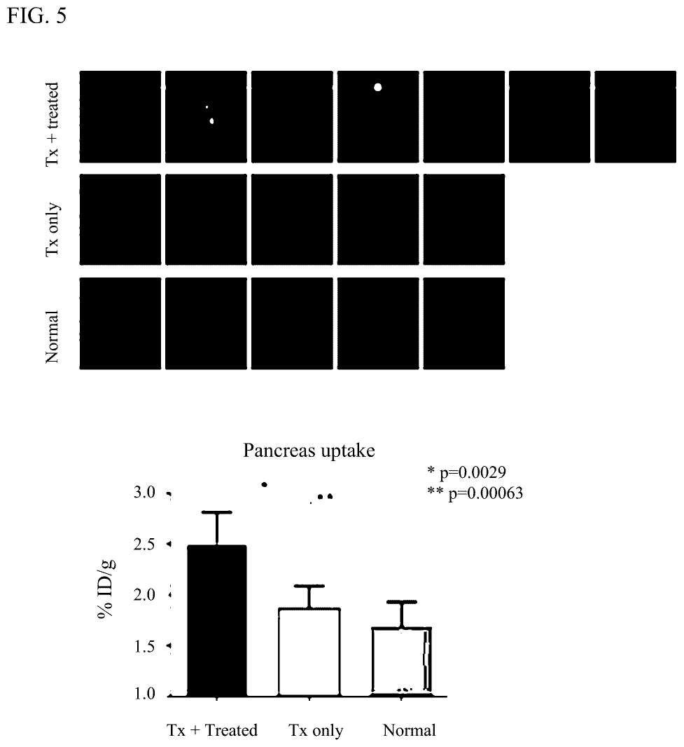

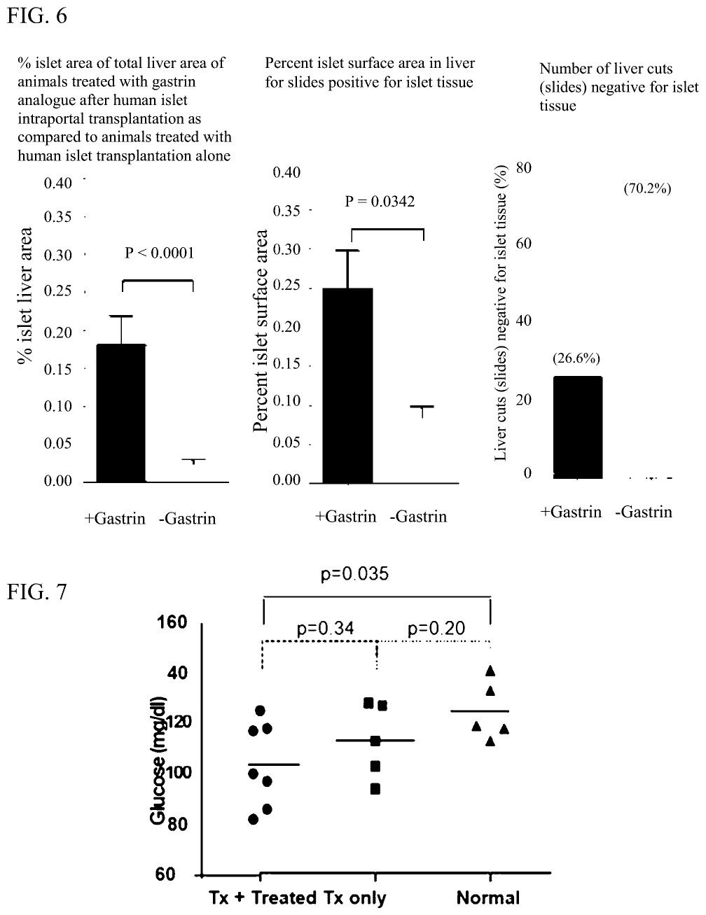

Isolated human islets were transplanted (Tx) to the livers of NOD mice followed by GAST-17 treatment for 30 days (150 μg/kg/dose, injected three times daily) (Tx+Treated, n=7) and compared to mice receiving islet transplant alone (Tx only, n=5) and untreated controls (Normal, n=5). After completion of treatment, whole mice and organs of interest were imaged (in vivo and ex vivo) with ¹⁸F-TC-Exendin-4 (TCE4) using microPET and a high specific activity labeling technique developed at COH for targeting islets (49). Compared with the control group, uptake by islet grafts located in liver of the GAST-17-treated group were significantly increased both in vivo (whole body images) (p=0.000015) (FIG. 3) and ex vivo (excised liver. p=0.000036) (FIG. 4), suggesting beta cell expansion. Also, ex vivo imaging of the pancreas showed significant expansion of native beta cell in GAST-17 treated animals (p=0.000063) (FIG. 5). Beta cell mass was calculated as percent beta cell area of the total cell surface area found on immunolabeled tissue sections. Data from 4 livers from mice treated with islet transplant alone (“−Gastrin”) and five livers from mice treated with islet transplant and gastrin treatment (“+Gastrin”) (˜32 slides per liver) were assessed. Ongoing preliminary analysis of the data suggests gastrin increased transplanted islet beta cell mass (FIG. 6). These data also support the increased in intensity of transplanted islet images shown above in the gastrin treated animals is related to an increase in beta cell mass. Collectively, these data provide evidence that adult human islets are capable of being expanded with gastrin treatment.

Gastrin treatment is associated with lower glucose levels.

Animals treated with human islet transplant and Gastrin-17 had lower blood glucose, as compared to untreated animals, while those treated with islet transplant alone had intermediate values (FIG. 7).

Human islets express the gastrin receptor CCKBR.

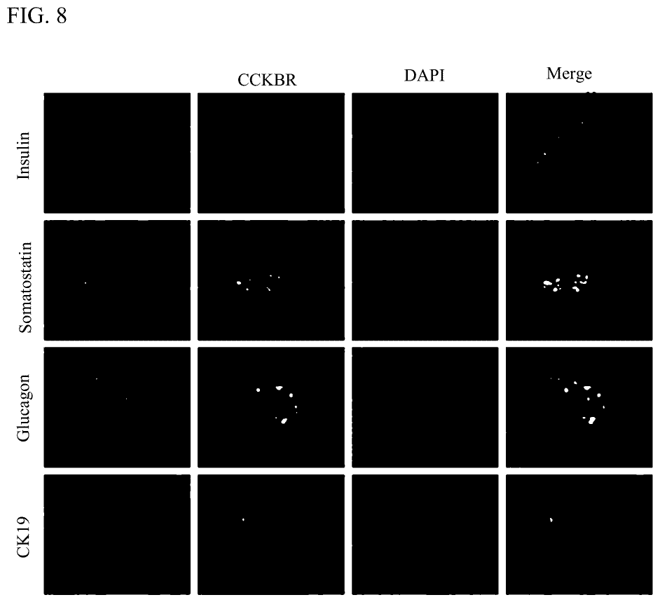

To investigate a possible GAST-17 effect on human islets, the gastrin receptor, CCKBR, was first localized in adult human islets by immunofluorescence staining (FIG. 8). Previous reports indicated that CCKBR is located on both delta and alpha cells. However, in the current study CCKBR co-localized with somatostatin expressing cells in pancreas slices from 10 donors (HbA1c 4.7-10.4), but not with glucagon, indicating that CCKBR is expressed mainly in delta cells.

Gastrin alters gene expression preferentially in islets from individuals with long-standing hyperglycemia.

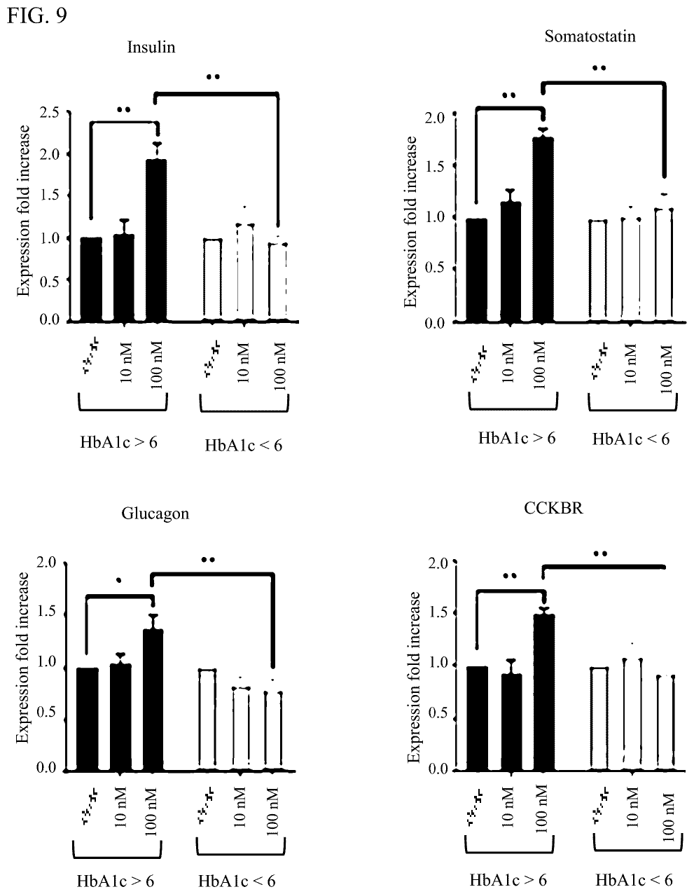

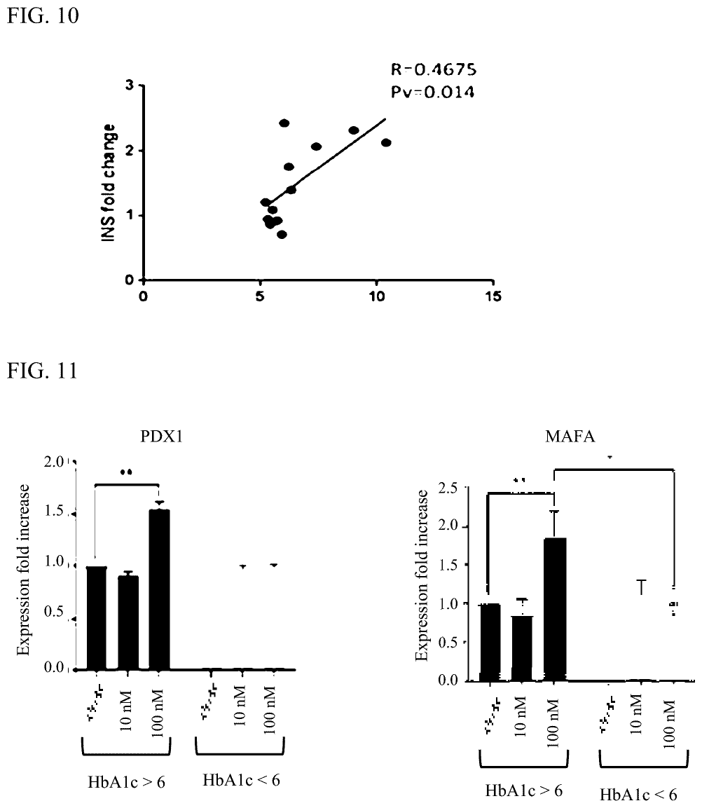

Isolated human islets from 11 donors with varied glycemic control (HbA1c 5.2-10.4) were treated with GAST-17 for 48 hours and gene expression was analyzed by qPCR. The results showed that the effect of gastrin was dependent on the HbA1c level of the islet donor, with HbA1c >6.0 (n=5) showing a significant increase in insulin (P<0.0001), somatostatin (P<0.0001) and glucagon (P<0.02) transcripts. Gene expression levels were not increased in islets from donors with an HbA1c <6.0 (FIG. 9). The increase in insulin mRNA correlated with the HbA1c levels, with higher insulin transcripts seen in islets from donors with more elevated HbA1c (FIG. 10). In addition, there were significant increases in the mRNA levels of known beta and delta cell transcription factors MAFA, MNX1, NKX2.2, NKX6.1, PDX1 and HHEX only in the HbA1c >6.0 group (FIG. 11).

Blockade of the gastrin receptor mitigates gene expression changes in human islets.

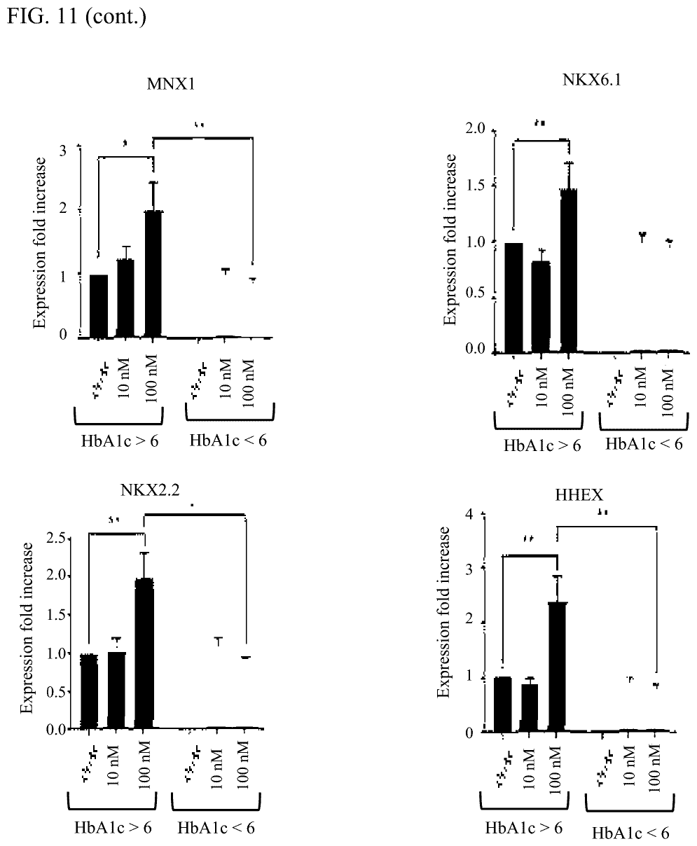

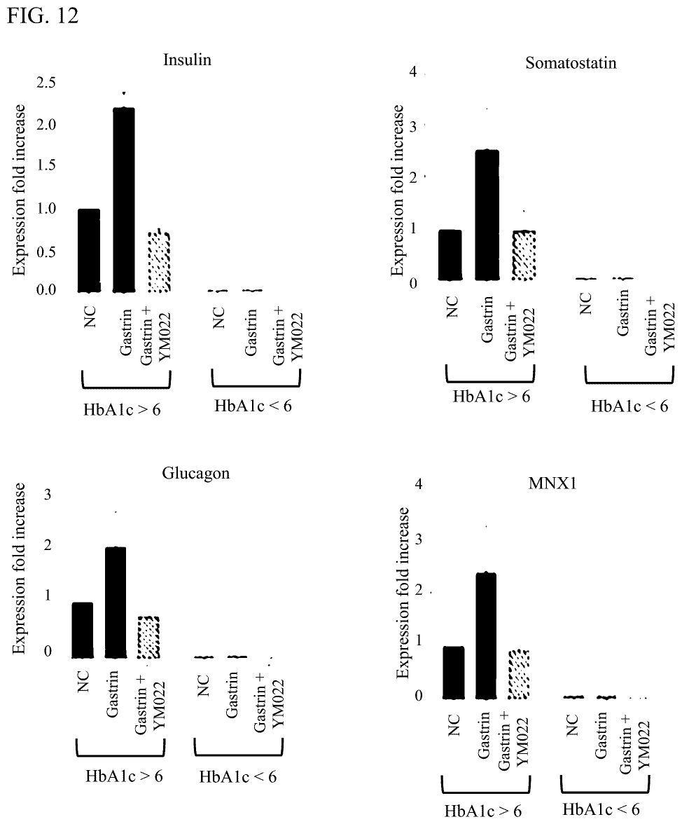

To establish that gastrin effect is mediated through activation of the gastrin receptor CCKBR, islets were treated with either 100 nM gastrin or 100 nM gastrin together with the CCKBR antagonist YM022. In islets from donors with high HbA1c levels, gastrin treatment again increased insulin mRNA by more than 2 folds, and somatostatin and glucagon mRNA by 2.5-fold and 1.8-fold, respectively. However, in islets treated with gastrin and YM022, insulin, somatostatin and glucagon mRNA levels remained un-changed. Additionally, in islets from healthy donors, gastrin±YM022 did not have any effect on mRNA levels of target genes (FIG. 12). Taken together, these data show that gastrin acts via CCKBR. These data add support to the idea that gastrin beneficially modifies islets post-transplantation.

Gastrin decreases inflammatory gene expression in hypoxic human islets.

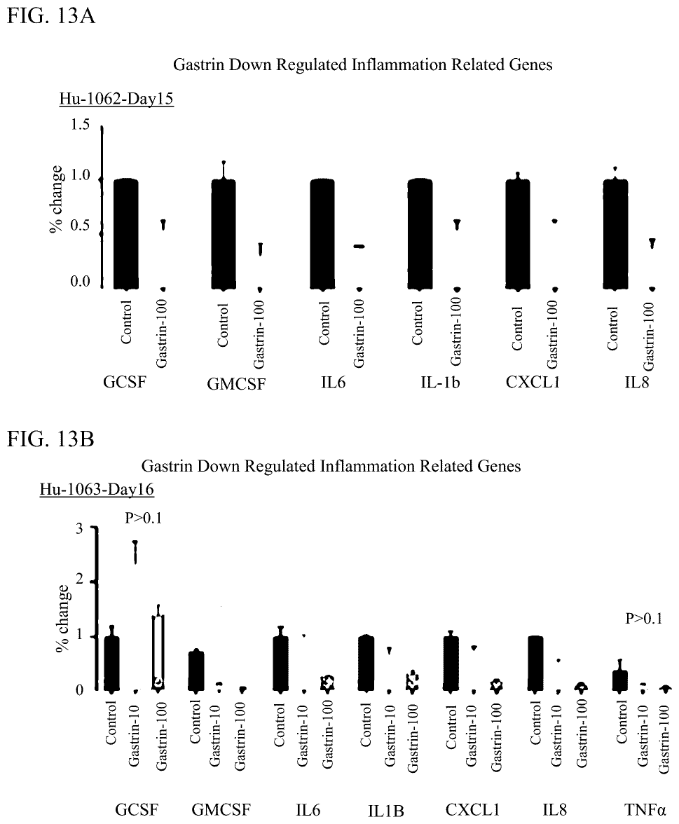

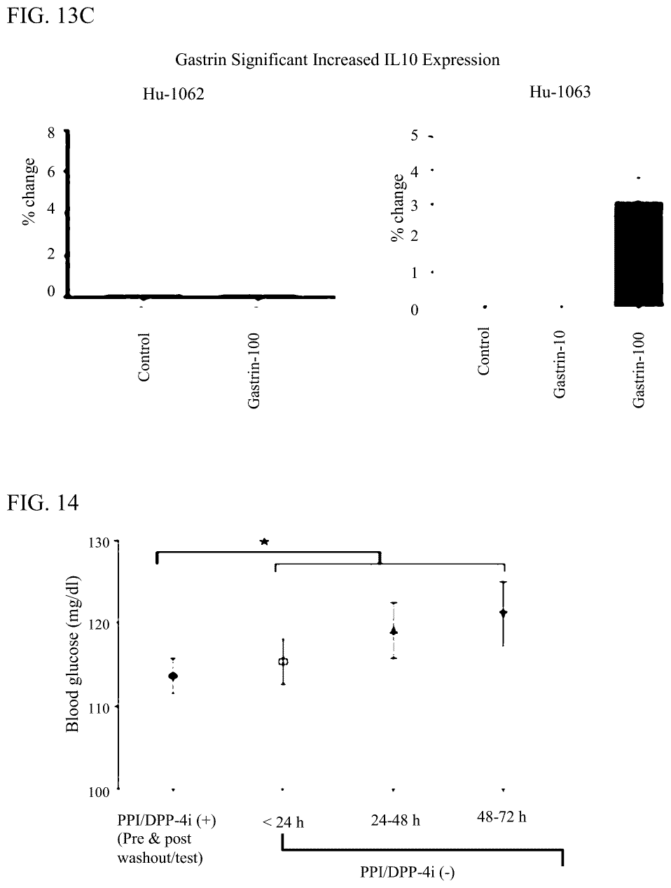

Long-term islet culture is associated with expression of inflammatory cytokines and islet stress/damage. Human islets from non-diabetic donors were cultured with and without GAST-17 long-term (1516 days) at normal oxygen concentrations (21%). GAST-17 treatment reduced islet expression of inflammatory genes (FIGS. 13A and 13B, below) and increased expression of IL-10 (FIG. 13C, below), suggesting a potential immune regulatory effect of GAST-17.

Proton pump and DPP-4 inhibitors support human islet function.

Treatment with GAST-17 may cause hypergastrinemia. Chronic hypergastrinemia is associated with a variety of clinical conditions, such as gastrinomas and atrophic gastritis. However, hypergastrinemia is generally well-tolerated in humans for many years if gastric acid secretion is inhibited using agents such as proton pump inhibitors (PPI). Interestingly, proton pump inhibitors have also been shown to increase plasma gastrin concentrations (41). Another oral class of medications, dipeptidyl peptidase-4 inhibitors (DPP-4i) are used for treatment of T2D. DPP-4 inhibitors increase active GLP, as well as gastric inhibitory polypeptide in the circulation, which in turn slows gastric emptying, reduces food intake and glucagon secretion, increases insulin secretion and may have beta cell protective effects (50). Combined treatment with PPI/DPP-4i has also been shown to induce beta cell expansion/neogenesis in NOD mice (51). For these reasons, PPIs and/or DPP-4 were proposed as adjunct treatments of patients with T1D (52-63). In fact, the current Islet Cell Transplant Program routinely uses PPI (esomeprazole) and DPP-4i (sitagliptin), for functional islet graft support. These agents are held for 3-7 days prior to metabolic studies to avoid drug-related confounding effects. Comparing self-monitored blood glucose readings during and off treatment with these agents demonstrates better glycemic control when these agents are used (FIG. 14).

Gastrin promotes multiple salutary effects on human islet beta cells.

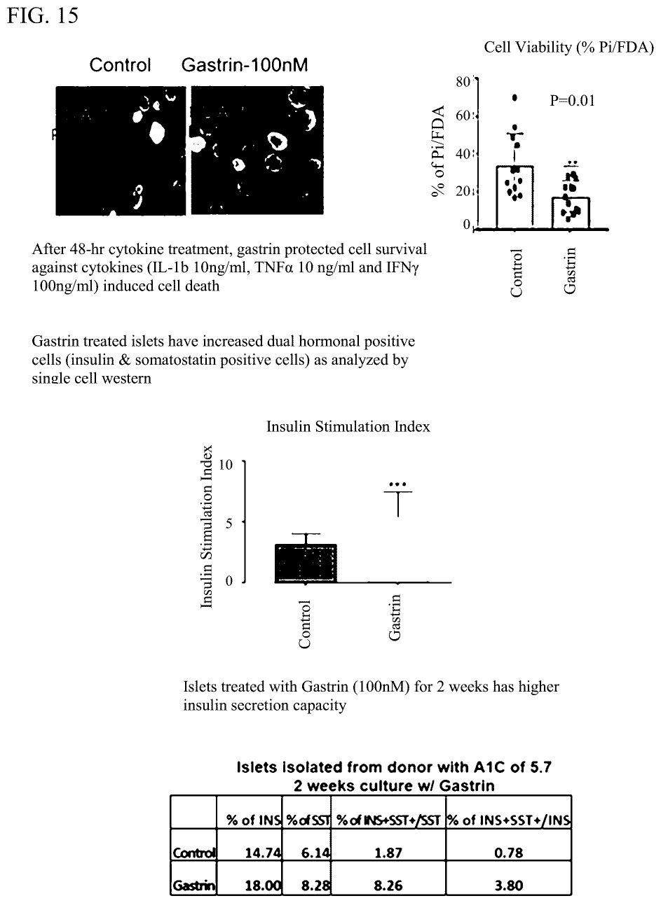

Gastrin is expressed in insulin⁺ and somatostatin⁺ cells in islets from people with T2D. As noted, gastrin increased insulin/somatostatin in delta cells, and this correlated positively with islet donor A1c levels (64). Extending these published and new findings, human islets were challenged with inflammatory cytokines (to mimic the harsh environment of transplantation) in the presence and absence of GAST-17 and glucose-mediated insulin secretion was determined. Interestingly, Gastrin reduced human islet damage from inflammatory cytokines, enhanced insulin secretion, and increased insulin+/somatostatin+ cell numbers (FIG. 15).

Gastrin improves islet transplant outcomes in individuals with T1D.

Initial evidence in 2 patients who underwent IT with gastrin found that insulin independence was achieved with a single islet transplant of <6,100 IEQ/kg (FIG. 16). In contrast, patients historically require multiple transplants and significantly more islets to achieve insulin independence. Average reduction of blood glucose per transplanted 1000 islet equivalents/kg in these two patients was 11.43 mg/dl vs. 4.88 mg/dl in 8 T1D recipients transplanted under identical T-cell depleting protocol without gastrin (Mean, P<0.001) over up to 1 year followed up period. Expansion of this trial will further confirm the highly encouraging preliminary studies with gastrin.

### Data Summary in Relation to Trial Design

The instant preliminary studies suggest that GAST-17 treatment of non-diabetic animals induces beta cell expansion/neogenesis. In vitro data with human islets also suggests that GAST-17 effects are mediated through the gastrin receptor CCKBR on the somatostatin+ cells which indicate that beta cell expansion/neogenesis may, in part, arise from delta cell trans-differentiation. Data with islets from diabetic/prediabetic donors also shows that this effect may depend on overall glycemic control and involve reprogramming of delta cells to insulin expressing cells. This provides a rationale for the clinical protocol wherein first 30-day course of GAST-17 is initiated shortly after islet transplantation when patients are more likely to have continuing hyperglycemia. A second 30-day course of GAST-17 treatment is repeated after 6 months. The 2^{nd} course of GAST-17 aims to evaluate islet responsiveness after full islet engraftment.

Aim: To test that GAST-17 treatment is safe, will improve islet engraftment, induce beta cell expansion/function, and enhance islet transplant outcomes in T1D individuals.

### Objectives:

1) Determine the safety of GAST-17 therapy in islet transplant recipients. Safety is evaluated by monitoring adverse events over a one-year follow-up period.
2) Determine that GAST-17 treatment improves islet transplant outcomes. The primary composite efficacy endpoint is the proportion of subjects who are insulin independent, severe hypoglycemia-free and have a HbA1c <6.5% at one-year post islet transplant (“complete response”). Secondary efficacy endpoints include the proportion of subjects who are severe hypoglycemia free and have a HbA1c <7.0% (“partial response”); reduction in hypoglycemic episodes, reduction in daily insulin use, and others. The trial also assesses changes in quality of life (QOL) after transplant to characterize the benefits of islet transplantation/gastrin therapy.
3) Determine that GAST-17 treatment induces beta cell expansion/neogenesis and/or enhances beta cell functional capacity in islet transplant recipients. Since clinical methods for measuring beta cell mass in people directly are not available, beta cell expansion/neogenesis and durability of GAST-17 effects are evaluated indirectly by comparing beta cell functional responses (C-peptide/insulin secretion in response to metabolic stimulation such as intravenous glucose/arginine infusion) within subjects at multiple time points before and through one-year post islet transplant. Since gastrin treatment is given for two courses of one month each (the first month and at month 6 post islet transplantation), continuing improvement in islet function between day 75 and 6 months after islet transplantation and between month 6 before initiating the second gastrin treatment course and at 75 days and 6 months afterwards (12 months post islet transplantation) supports the possibility of beta cell expansion/neogenesis as a result of gastrin administration. Days from transplant to discontinuation of insulin intake while maintain blood glucose at target are determined and compared to the same parameter in other protocols. Also, glucose variability in this and a similar protocol without use of gastrin are determined and compared.
4) Identify novel biomarkers that predict islet function after transplantation. The trial also collects blood samples to identify circulating islet related genomic material, prevailing immune milieu and other novel biomarkers that may help predict islet transplant outcome and/or islet graft dysfunction/rejection.

### Study Design

This is a Phase I/II, prospective, single arm, single site trial to assess the safety and efficacy of islet transplantation using T-cell depleting immunosuppression induction and two 30-day courses of GAST-17 with long-term PPI and DPP-4i oral therapy in T1D subjects with unstable glycemic control. A total of twenty T1D individuals with unstable blood glucose control who meet the inclusion/exclusion criteria (below) are included.

This trial seeks to establish the safety and efficacy of islet transplantation in combination with gastrin treatment to enhance insulin producing capacity of the islet graft, and thereby induce metabolic stability and allow achievement of insulin sufficiency with smaller transplanted islet mass. Detailed metabolic studies allow characterization of islet graft functional changes over time and immunologic/biomarker studies facilitate a better understanding of the interplay between immune and other mechanisms contributing to islet graft dysfunction/rejection. Finally, QOL is assessed over the course of the study to characterize the benefits of islet transplantation/gastrin therapy.

Target Study Population: This trial recruits adults with type 1 diabetes complicated by frequent hypoglycemia and/or hypoglycemia unawareness or otherwise unstable blood glucose control that satisfy the following study eligibility criteria.

### Inclusion Criteria:

Age 18-68 years;
Type 1 diabetes mellitus (documented with fasting C-peptide level of <0.2 ng/ml before and <0.3 ng/ml after IV administration of 1 mg of glucagon) for at least 5 years;
Unstable blood glucose control characterized by: Frequent hypoglycemia (blood glucose ≤54 mg/dl more than once per week), and/or—Hypoglycemia unawareness (Clarke score of 4 or more), and/or—One or more severe hypoglycemic episodes in 12 months preceding enrollment. Severe hypoglycemia is defined as an event with one or more of the following symptoms: memory loss, confusion, uncontrollable behavior, irrational behavior, unusual difficulty awakening, suspected seizure, seizure, loss of consciousness, or visual symptoms, in which the subject was unable to treat him/herself and which was associated with either a blood glucose level <54 mg/dl or prompt recovery after oral carbohydrate, IV glucose, or glucagon administration (67), and/or—Erratic blood glucose levels that interfere with daily activities, defined as one or more of the following: Glucose Variability Percentage >50 from continuous glucose monitoring, Patient self-report on ICT Candidate Application or Symptom Checklist that diabetes/blood glucose limits daily activities or employment, Diabetes Distress Scale—score of 3 or more in two or more of the following domains: Emotional Burden, Regimen-Related Distress, and/or Interpersonal Distress, and/or—One or more hospital visits for diabetic ketoacidosis in the 12 months preceding enrollment

Ability and willingness to comply with post-transplant regimen, including immunosuppression, use of reliable contraception, frequent clinic visits, testing and maintaining detailed logs of blood glucose levels, insulin doses and medications, and completing detailed follow-up studies.

### Exclusion Criteria:

BMI >33;
Insulin requirements >1.0 units/kg/day;
Significant kidney disease (estimated GFR from serum creatinine measurement <65 ml/min, random spot urine microalbumin to creatinine ratio >300 mg albumin/g creatinine);
Significant hepatobiliary disease, including elevation of liver enzymes >twice the upper limit of normal for each of ALT and AST, bilirubin not within normal limits, albumin <3.5 g/dl, liver masses, portal vein thrombosis, evidence of portal hypertension, or significant, untreated gallbladder disease (i.e. gallstones);
Significant cardiovascular disease, including non-correctable coronary artery disease with ejection fraction <50% and/or recent myocardial infarction (within last 12 months); or extensive peripheral vascular disease not correctable by surgery;
Evidence of active proliferative retinopathy;
Hypertension (>140/90) despite appropriate treatment;
Hyperlipidemia (total cholesterol >260 mg/dl, LDL >160 mg/dl, and/or triglycerides >300 mg/dl) despite appropriate treatment;
Anemia (Hgb <11 g/dl) or other hematologic disorders that require medical attention;
WBC <3,000/μl;
Increased risk of bleeding (platelet count <120,000 cells/μl; INR >1.5), other chronic hemostasis disorders, or treatment with chronic anticoagulant therapy (i.e. heparin or warfarin);
Recent unresolved acute infection (except for mild skin infection or nail fungal infection), or chronic infection, including tuberculosis, HIV, HBV, HCV, CMV or syphilis (RPR);
EBV IgG negative;
Any history of malignancy, except completely resected squamous or basal cell skin cancer or in situ cancer of the cervix;
Evidence of active peptic ulcer disease;
History of gastric bypass;
Recent history of non-adherence to recommended medical therapy;
Psychiatric illness that is untreated, or likely to interfere significantly with study compliance despite treatment;
Previous organ/tissue transplant;
Presence of preformed antibodies on panel reactive antibody screening >20%;
Administration of live attenuated vaccines within 60 days of enrollment;
Presence of a chronic disease that must be chronically treated with one or more of the following medications: glucocorticoids (unless for adrenal replacement), aspirin, non-steroidal anti-inflammatory agents (NSAIDs), diazoxide, haloperidol, chlorpromazine, desipramine, doxepin, imipramine, isoproterenol, levodopa, morphine, L-asparaginase, cyclophosphamide, isoniazid, heparin, nalidixic acid, or any other agents that may adversely influence glycemic control or confound the interpretation of study results. In order to reduce risk for bleeding, aspirin should not be given when subject is active on the wait list until transplant completed;
Use of investigational agents within four weeks of enrollment;
Active alcohol or substance abuse, including cigarette smoking (must be abstinent for >3 months);
Pregnant women, women intending future pregnancy, women of reproductive potential who are unable or unwilling to follow effective contraceptive measures (i.e., tubal ligation, two barrier methods, abstinence) for the duration of study treatment and for as long as they are on immunosuppressive medication, and women presently breast feeding are ineligible due to the unknown risks of study drugs on the fetus and nursing infant;
Individuals without health insurance covering the cost of immuno suppression and clinical and laboratory follow-up after completion of the study;
Or, any medical condition that in the opinion of the investigator will interfere with safe participation in the trial.

### Treatment:

A single allogenic islet transplant, infused intraportally and two-30 day courses of GAST-17, administered as subcutaneous injections twice daily. Overview of the two treatment courses are provided below.

Treatment course 1. Subjects receive a single islet infusion with T-cell depleting immunosuppressive induction (rATG or alemtuzumab), double anti-inflammatory blockage (etanercept and anakinra), and long-term immunosuppression (tacrolimus/MMF, with sirolimus added at 8 wks), and a 30-day course of subcutaneous GAST-17 starting approximately 2 days after islet transplant. Oral administration of DPP-4i (sitagliptin) and PPI (esomeprazole) is started with the first course of GAST-17. Subjects are monitored for adverse effects and assessed for preliminary efficacy at 1, 2.5, and 6 months after starting the first course of GAST-17.

Treatment course 2. Although IT and GAST-17 treatment can lead to insulin independence shortly after initiation of treatment, a second 30-day course of GAST-17 is initiated at 6 months post transplantation, in order to achieve and maintain glycemic stability and insulin independence. Subjects continue oral DPP-4i and PPI treatment throughout. The second course of treatment is not be given until 6 months after the transplant to allow time for islet engraftment and the assessment of maximum benefits from the first course of GAST-17 treatment. Outcomes are assessed at Months 1, 2.5, and 6 from the beginning of GAST-17 Treatment Course 2, as described above.

Follow-up: Subjects are followed for 1 year from the transplant (6 months following initiation of the second course of GAST-17) to evaluate the safety and efficacy of study treatment. Treatment efficacy is evaluated based on changes in daily insulin requirements and glycemic control, as well as through metabolic studies to quantify the insulin secretory capacity of the islet graft based on the endpoints described below (see Statistics and Data Analysis).

Follow-up Assessments: Subjects are followed for 1-year post islet transplant and the first course of GAST-17 treatment as described below.

### Assessment of Safety

Subjects are closely monitored for adverse events related to islet transplantation, immunosuppression, and gastrin treatment. Immunosuppressive induction, intraportal islet transplant and the initiation of the first course of gastrin are conducted during the hospital admission and under close monitoring. Subjects continue to be assessed in the outpatient clinic weekly for the first month and at days 75, Month 4 and Month 6 post the first gastrin course and at Month 1, Month 2.5 and Month 6 post the second gastrin course. Outpatient visits include review of symptoms, vitals/weight/BMI, review of blood/glucose and insulin logs, physical exam, lab assessments (CBC, biochemical, viral and other parameters), and assessment for changes in diabetes complications (urine protein excretion, neuropathy, retinopathy by fundoscopic exam).

Adverse event collection: All adverse events reported or observed since the time of the last clinic visit are recorded and graded according to the Clinical Islet Transplantation Consortium Terminology Criteria for Adverse Events (CIT-TCAE Version 5, Aug. 3, 2011). Safety stopping criteria are in place if Grade 3 or higher adverse events associated with gastrin therapy are observed (see Statistics, below).

### Assessment of Graft Function

Evaluations to assess islet graft function and changes in the insulin secretory response of the islet graft following each GAST-17 course include the following.

### Rate and Duration of Insulin Independence.

Insulin data is obtained from insulin pump downloads (if available) or from data self-reported by the subject at each Safety Monitoring visit (see Section 5.7.6). Pre-transplant daily insulin requirements is calculated as the average total units of insulin per day the subject used during the two weeks prior to islet transplant. If for any reason, data during this period is incomplete, data collected closest to the time of the first transplant is used. Official analyses to measure reduction in daily insulin requirements from baseline is done at Month 1 (Day +30±5), Month 2.5 (Day +75±14), and Month 6 (Day +180±14), post each GAST-17 course. Average insulin requirements is calculated as the average daily insulin requirement over the two weeks preceding the study time point.

### Glucose-Potentiated Arginine Stimulation Test (IVGTT+AST).

Insulin secretory capacity of transplant islets is assessed by IVGTT+AST at Month 2.5 (Day +75±14), and Month 6 (Day +180±14) post each GAST-17 course. The study begins with the COH ICT Program's standard intravenous glucose tolerance test (IVGTT). Briefly, two baseline samples are drawn for glucose, insulin, C-peptide and glucagon, levels over 10 min. Then 50% dextrose (300 mg/kg) is given intravenously over 1 min. Nine samples are drawn during the following 30 min for glucose, insulin, C-peptide and glucagon determinations at 3, 4, 5, 7, 10, 15, 20, 25, and 30 min, with 0 time being defined as the beginning of the infusion. The arginine stimulation test (AST) is initiated immediately following the 30 min IVGTT blood draw. Briefly, within 5 min post the 30 min IVGTT blood draw, 5 g intravenous bolus of arginine (L-arginine HCl 10%) is administered over 30-60 seconds. Zero time for the AST is defined as the beginning of arginine infusion. During the following 30 minutes, ten samples is drawn at 2, 3, 4, 5, 7 10, 15, 20, 25, and 30 min for measurement of glucose, insulin, C-peptide and glucagon concentrations. IVGTT+AST data is analyzed for acute insulin response to glucose (AIR_{g}), glucose disposal (KG), and area under the curve (AUC) for glucose (AUCg), insulin (AUCi), C-peptide (AUC_{C-p}) and glucagon (AUC_{G}) is assessed. The AUCg, AUCi, AUC_{C-p}, and AUCg is calculated over the full study and represents the area above the baseline. Insulin sensitivity is assessed using the homeostasis model assessment (HOMA) as an estimate of insulin sensitivity based on fasting glucose and insulin levels (68). Maximal stimulation of insulin secretion after arginine administration is examined.

### HbA1c.

HbA1c is measured pre and at Month 2.5 (Day +75±14) and Month +6 (Day +180±14) post the start of each GAST-17 treatment course to track improvements in glycemic control. A HbA1c of 6.5% is targeted.

### Glucagon Stimulation.

An intravenous glucagon stimulation test is done at Month 1 (Day +30+5), Month 2.5 (Day +75±14) and Month 6 (Day +180±14) post the start of each GAST-17 treatment course. Briefly, after an overnight fast, a baseline blood sample is drawn to measure fasting C-peptide, glucose, insulin and proinsulin levels as well as serum creatinine (alternatively, serum creatinine measurement can be taken from CMP report if drawn same day). Glucagon (1 mg) is administered intravenously and a post-stimulation blood sample is drawn at six minutes to measure glucagon-stimulated C-peptide, glucose, insulin and proinsulin levels. The C-peptide to glucose, creatinine ratio (CPGCR) is calculated from the fasting sample. This measure accounts for both the dependence of C-peptide secretion on the ambient glucose concentration and the dependence of C-peptide clearance on kidney function. The CPGCR is calculated as [C-peptide (ng/ml)×100]/[glucose (mg/dl)×creatinine (mg/dl)]. This study has been adopted from the metabolic follow-up studies being performed by the NIH-supported Collaborative Islet Transplantation Consortium. The ratio of insulin to proinsulin is assessed as an indicator of islet stress (69-74).

### Modified Oral Glucose Tolerance Test (OGTT).

A modified OGTT is done at Month 1 (Day +30+5), Month 2.5 (Day +75±14) and Month 6 (Day +180±14) post the start of each GAST-17 treatment course to monitor plasma glucose, insulin, and c-peptide levels before and at 120 minutes after ingestion of a glucose beverage according to ICT SOPs. Subjects report to clinical after an overnight fast. Basal glucose, insulin, and c-peptide levels are drawn. Immediately after, the subject receives a glucose solution (Glucola® drink or equivalent substitute: 75 g of glucose dissolved in 225 ml of water) to consume in 5 minutes starting at time=0. Then, at time=120 minutes, stimulated glucose, insulin, and c-peptide levels is drawn. OGTT may be done at additional time points at PI discretion, if islet graft dysfunction is suspected.

### Continuous Glucose Monitoring (CGM).

Continuous glucose monitoring is performed for 3 or more consecutive days once prior to islet transplant, and at Month 1 (Day +30+5), Month 2.5 (Day +75±14) and Month 6 (Day +180±14) post each GAST-17 treatment course, and at additional time points as needed if islet graft dysfunction is suspected, using a commercially available subcutaneous continue glucose sensor. These sensors measure interstitial fluid glucose levels continuously, both pre and post-prandially. These readings have a good correlation with capillary glucose measurements and are useful as a basis for measuring shifts in tissue glucose levels. The data from the sensors is downloaded into a computer program, where an integrated interpretation of daylong glucose levels can be calculated. The Glycemic Variability Percentage (GyP) can be calculated as described by Peyser et al (75). If a subject uses a CGM as part of their normal diabetes management plan, data from the subject's personal CGM device may be used instead of connecting a separate CGM device.

### Glycemic Control Surveys/Assessments.

The following validated glycemic control surveys and assessments is analyzed prior to treatment and on Month 1 (Day +30+5), Month 2.5 (Day +75±14), Month 6 (Day +180±14) post start of each GAST-17 treatment course.

Ryan Hypo Score. Composite indices of hypoglycemia frequency, severity, and symptom recognition is assessed by the HYPO score (76). The HYPO score involves subject recording of BG readings and hypoglycemic events (BG<54 mg/dL) over a 4-week period and recall of all severe hypoglycemic episodes in the previous 12 months. A HYPO scores greater than or equal to the 90th percentile (1047) of values derived from an unselected group of T1D subjects indicates severe problems with hypoglycemia.

Glycemic Lability Index. The Glycemic Lability Index (LI)(76) requires 4 or more daily capillary BG measurements over a 4 week period and is calculated as the sum of all the squared differences in consecutive glucose readings divided by the hours apart the readings were determined (range 1 to 12 hours) in (mmol/12) hr⁻¹·wk⁻¹. A LI greater than or equal to the 90th percentile (433 mm2 hr⁻¹ wk⁻¹) of values derived from an unselected group of T1D subjects is evidence of severe glycemic lability.

Clarke Survey. Composite indices of hypoglycemia frequency, severity, and symptom recognition are assessed by the Clarke Survey (77). The Clarke survey involves subject completion of 8 questions scored according to an answer key that gives a total score between 0 and 7 (most severe), where scores of 4 or more indicated reduced awareness of hypoglycemia and increased risk for severe hypoglycemic events.

Mean Amplitude of Glycemic Excursions. The extent of glycemic lability is assessed using MAGE (78). The MAGE requires 14-16 capillary BG measurements over two consecutive days taken before and 2 hours after breakfast, lunch, and dinner, and at bedtime with an optional measurement at 3 a.m. A glycemic excursion is calculated as the absolute difference in peak and subsequent nadir (or vice versa) glucose values, with the direction (peak to nadir versus nadir to peak) determined by the first quantifiable excursion in the two-day period. All excursions >1 S.D. of the 7-8 glucose readings for the day in which they occurred qualify for the analysis, where they are summed and divided by the number of qualified excursions to give the MAGE in mg/dl glucose. A MAGE >200 mg/dl is indicative of marked glycemic lability.

Composite assessments of islet graft function. The following composite assessments of islet graft function is analyzed at Month 1 (Day +30+5), Month 2.5 (Day +75±14), Month 6 (Day +180±14) post start of each GAST-17 course.

Ryan Beta Score. The Beta-score is determined using HbA1c, insulin requirements, fasting glucose and basal or stimulated c-peptide per Ryan et al (79). The score may range from 0 (no function) to 8, with all subjects reported with a score of 8 also having 90-minute glucose levels during MMTT ≤180 mg/dl, indicative of excellent graft function.

City of Hope Model for Islet Therapy and Islet Scoring (MITRIS). Data collected were also evaluated using a computer-based algorithm developed at COH, which is specially designed to analyze multiple pre- and post-transplant subject parameters to predict insulin requirements post-islet transplantation (80). The algorithm is used as a supplement to help guide post-transplant blood glucose management and assess for islet graft dysfunction.

### Assessment of Quality of Life

Islet transplant has been shown to positively impact the QOL of patients with T1D (81, 82). This effect appears to be the result of improved glycemic stability and reduction in anxiety related to hypoglycemia. QOL is assessed at the time of study qualification, on Day 0 (unless done within preceding 3 mo), at Month 2.5 (Day +75±14) and Month 6 (Day +180±14) post the start of GAST-17 Course I and Month 6 (Day +180±14) post the start of GAST-17 Course II to determine short- and long-term changes in physical, emotional, and social wellbeing. Four fully validated QOL assessment tools designed specifically for subjects with diabetes or assessment of general health-related QOL are used. These QOL assessments are the same utilized by the NIH-sponsored Clinical Islet Transplant Consortium (CIT) and the Clinical Islet Transplant Registry (CITR) (83).

Diabetes Distress Scale (DDS): This is a 17-item self-administered questionnaire [163]. The DDS measures four diabetes-related distress domains: emotional burden (EB), physician-related interpersonal distress (PD), regimen-related distress (RD), and diabetes-related interpersonal distress (ID). Per the developers, a mean item score of 3 or higher in any one domain is considered “moderate distress” and is interpreted as evidence of that glycemic control is interfering with daily activities.

EQ-5D (EuroQoL): The EQ-5D is a public domain instrument (see World Wide Web site at euroqol.org) that generates a descriptive profile and single index value for health status. The descriptive portion addresses five health dimensions (mobility, self care, usual activities, pain/discomfort, and anxiety/depression) with respondents indicating one of three possible responses for each dimension. Summary data can be reported as the proportion of respondents with problems in each dimension. Additionally, the multidimensional “health state” can be converted to a single weighted health status index that reflects the valuation of various possible health states from general population samples, including one that has been developed in a nationally representative US sample. The second portion of the EQ-5D is a (0-100) visual analogue scale that is used to report overall health status. Advantages of this instrument include its brevity and particular application in cost-effectiveness research.

Hypoglycemic Fear Survey II (HFS-II): The HFS-II [164] is a 33-item scale designed to quantify patient fear related hypoglycemia. The scale consists of 15 items to evaluate the subject's use of hypoglycemia avoidance behaviors (e.g., eat large snack) and 18 items to evaluate the subject's level of worry about hypoglycemia (e.g., frequency at which the subject worries about having a hypoglycemic episode while driving). Subjects respond on a 5-point Likert scale (Never, Rarely, Sometimes, Often and Always). A response of “Never” indicates that the subject “Never” uses the specified avoidance behavior or “Never” worries about the specified worry parameter. A response of “Always” indicates that the behavior/worry is experienced “Always.”

RAND SF-36v2™ Health Survey: This Health Survey is a 36-item instrument for measuring general health status and outcomes from the subject's point of view. The SF-36v2™ measures eight health concepts, including, 1) limitations in physical activities due to health problems; 2) limitations in usual role activities due to physical health problems; 3) bodily pain; 4) general health perceptions; 5) vitality (energy and fatigue); 6) limitations in social activities because of physical or emotional problems; 7) limitations in usual role activities because of emotional problems; and 8) mental health (psychological distress and well-being). The SF-36 uses a variety of question types, including rankings according to a 5-6 point scale and simple Yes or No answers. Responses for each item are assigned a score ranging from 1-100. Scores represent percentage of total possible score achieved. The scores under each of the 8 health concept areas are averaged together to create 8 scale scores. A high score represents a more favorable health state.

### Immune Monitoring

Immune activation is investigated to increase understanding of the immunologic causes of islet graft rejection and for immunomodulating effects of GAST-17 treatment. Unless otherwise specified, the allo- and autoimmune studies described below are performed in sequential blood samples taken from islet recipients pre-transplant and at Month 2.5 (Day +75±14) and Month +6 (Day +180±14) post start of GAST-17 Couse I, at Month 6 (Day +180±14) post start of GAST-17 Course II, and if/when islet graft dysfunction/rejection is suspected. Results from these assays are related to immunosuppression and other immunological and clinical parameters (e.g., graft rejection, rate/duration of insulin independence and endpoints of graft function including insulin requirements, C-peptide levels, HbA1c, IVGTT/OGTT results, etc).

Cytokine Analysis. Changes in the following serum cytokines associated with Th1, Th2 and inflammatory cells are monitored by fluorochrome technology (Luminex) pre-transplant, at Month 1 (Day +30+5), Month 2.5 (Day +75±14) and Month 6 (Day +180±14) post start of GAST-17 Course I, at Month 1 (Day +30+5) and Month 6 (Day +180±14) post start of GAST-17 Course II, and if/when islet graft dysfunction is suspected: GCSF, GMCSF, TNF-α, TGF-β1, PDGF, IL-1f3, IL-5, IL-6, IL-7, IL-8, IL-10, IL-12, IL-13, IL-15, IL-17, IL-33, IFN-α, CXCL10, CCL4, and CCL5. The cytokines to be monitored can vary based on availability of reagents.

Immune Cell Panels. Peripheral blood mononuclear cells (PBMCs) are analyzed by flow cytometry to track changes in immune cell populations before and after islet transplant with GAST-17 treatment. Composition (percent and absolute counts) of B-cell, monocyte, natural killer (NK) cell, T-cell subsets are determined.

ImmuKnow Immune Cell Function Assay. Monitoring the patient's global immune response has the potential to provide important information on the patient's individual response to drugs and allows a mechanism for the tapering of drugs and monitoring efficacy of interventional therapies. ImmuKnow® assay is a simple whole blood assay that has FDA clearance to measure global T cell immune responses in immunosuppressed individuals. The assay detects cell-mediated immune responses in whole blood after a 1518 hours incubation with phytohemagglutinin (PHA). Data produced by the UCLA Immunogenetics Center [74] show that the ImmuKnow assay has predictive value and provides a target immunological response zone for minimizing risk and managing subjects to stability. It is to be determined if a longitudinal study of the transplant recipient's global immune response using the ImmuKnow assay is a valuable tool to directly assess the “net state” of immune function of the islet transplant recipient for better individualizing therapy (84).

Flow Cytometry Cytokine Secretion (FCCS) assay for donor-specific lymphocyte activation. Previous studies have shown that the presence of T cells activated via the direct and indirect pathway in the peripheral circulation of allograft recipients correlates significantly with rejection (85-93). A study by Roep et al analyzed cytolytic T lymphocyte (CTL) and T helper cell precursor frequency to graft specific alloantigens in recipients of human islet grafts implanted in the liver of immunosuppressed T1D patients (94). The results of the study showed that rapid failure of islet allografts was accompanied by an increased frequency of donor specific alloreactive T cells. In contrast, the patients who remained C-peptide positive for >1 year did not exhibit signs of alloreactivity. To monitor alloreactive T cells in the circulation of transplant recipients, T cell reactivity to donor cells and donor cell free membrane antigen preparations in sequential samples of blood were evaluated using the flow cytometry cytokine secretion assay. The flow cytometry cytokine secretion assay (FCCS) represents a sensitive assay to enumerate antigen-specific responses of memory T cells (85-93). The method is based on the detection of cytokines produced by a single cell within a polyclonal population using cell surface affinity matrix technology. This assay is used to determine the frequency of direct and indirect donor-specific alloreactive T cells and to correlate this information with other immunological, metabolic and other clinical parameters and with recipient genomic profile.

Anti-HLA antibody ID for assessment of humoral immune response to donor. Anti-HLA class I and/or class II antibodies are determined by assessing reactivity against a panel of single recombinant HLA antigen preparations with flow PRA testing (when indicated). Results are compared to islet cell transplant outcomes to evaluate whether alloantibody production precedes, accompanies, or follows episodes of rejection. Correlation between antibody production and impact on long term islet graft survival are assessed.

Detection of Autoimmune Reactivation. Reactivation of autoimmune disease is another potential immunologic pathway that may lead to islet graft rejection. Reactivation as measured by insulin and islet cell autoantibodies have been noted to a limited extent by other islet transplant groups (13, 29, 30). Results are correlated with data from the alloimmunity studies, as well as with other metabolic and clinical parameters.

Serum autoantibodies: Islet rejection due to recurrence of autoimmunity is monitored by detecting the presence/levels of antibodies directed against insulin (insulin autoantibody; IAA), islet cells (IA-2), glutamic acid decarboxylase (GAD65), and a zinc transporter involved in insulin maturation and storage in the pancreatic beta cells (ZnT8). These antibodies are considered markers for autoimmune islet destruction in patients with type 1 diabetes (95, 96). Thus, the time course of any increase in levels autoantibodies relative to islet transplantation and islet function is examined to determine the recurrence of anti-islet autoimmunity.

Autoreactive memory T cells: Detection of autoreactivity recurrence in islet transplant recipients is also monitored at baseline (before treatment) and at Month 6 post start of second course of GAST-II (Day +180±14 days) and as deemed appropriate if islet graft dysfunction is suspected. Assays are performed to examine memory T cells specific for T1D autoantigens.

### Assessment of Gene Expression and Other Biomarkers

Recent advances in genetic and epigenetic profiling have made it possible to characterize mechanisms underlying diabetic changes and islet function, such as those noted by Weir et al in response to glucose toxicity (97, 98). The following studies are conducted to characterize changes in gene expression and other biomarkers before and after treatment.

Recipient genomics. To assess the effect of the proposed combination therapy, changes in gene expression are monitored before and after treatment in peripheral blood samples. Whole blood samples (11.5 ml) are collected pre-transplant, at Week 1 (Day +7±3) and at Month 1 (Day +30+5), Month 2.5 (Day +75±14), and Month 6 (Day +180±14) post start of GAST-17 course I, at Month 6 (Day +180±14) post start of GAST-17 course II, and when islet graft dysfunction/rejection is suspected. Gene expression is analyzed. The time course of sampling has been chosen to monitor the pre-transplant profile, immediate post-transplant period, including the effect of immune induction with rATG, transitions in maintenance immunosuppression, and GAST-17 treatment. Finally, genomic assessment at times of islet graft dysfunction can identify which genes if any, become activated during periods of islet graft dysfunction/rejection. (99-103)

BI-PAP-A assay for measurement of circulating islet DNA. A major difficulty in islet transplantation is monitoring graft health. Typically, this is done by following changes in metabolic parameters (blood glucose and C-peptide levels and insulin requirements). However, such evidence may not appear until significant damage to the graft has already taken place. The transplant group in Geneva has developed a method to measure loss of cells from the islet graft directly using reverse-transcription polymerase chain reaction (RTPCR) to measure insulin messenger RNA (mRNA) in the circulation of islet recipients as an indicator of islet graft damage (99). One concern with this approach, however, is the lability of RNA in blood, which may compromise the ability to detect cell loss. To address this problem, a new method is employed to measure donor DNA using a sensitive assay developed at COH, as DNA has a much longer resident time in the circulation (several weeks to over Imo) (100-102).

Bidirectional Pyrophosphorolysis-Activated Polymerization Allele-Specific Amplification (BI-PAP-A) assays is done pre-transplant, on Days +1 and +2 post islet transplant, at Week 1 (Day 7±3), Week 2 (Day +14±3), Month 1 (Day +30+5), Month 2.5 (Day +75±14), and Month 6 (Day +180±14) post start of GAST-17 Course I, at Month 6 (Day +180±14) post start of GAST-17 Course II, and if islet graft dysfunction is suspected. Results are compared and correlated with clinical outcomes to determine their ability to meaningfully predict islet graft loss. Briefly, peripheral blood samples are collected from islet recipients at the time points listed above. DNA is isolated independently from the cell and plasma fractions, and BI-PAP-A assays are performed as previously described (104) using reagents determined to be specific for donor tissues by previous analysis of donor samples. Preliminary data using this method in islet recipients was able to detect donor-specific gene snips in the peripheral blood in the early post-transplant period (reflecting expected islet graft loss due to immediate blood mediated reactions) and when islet injury as occurred as a result of allo/auto-islet rejection (data in preparation).

Quantitative Methylation-Specific Polymerase Chain Reaction (qMSP) assay for circulating beta cell DNA. There has been rising interest in use of demethylated insulin gene as a marker of in vivo islet destruction. Investigators have successfully developed a quantitative methylation-specific polymerase chain reaction (qMSP) assay for circulating beta cell DNA to monitor the loss of beta cells. The assay was based on the premise that the insulin gene while present in all tissues, is unmethylated in insulin-producing cells (islets) but methylated in other tissues. Therefore, presence of the demethylated insulin genes in the peripheral circulation may be useful in identifying beta cell destruction from either the native pancreas in new onset diabetes or from transplanted islets. It was recently reported that this assay detects the rise in circulating beta cell DNA in the early post-transplantation period in islet recipients (105, 106). Thus, the utility of the demethylated insulin gene signature assay as tool for detecting islet graft injury in islet transplant recipients is to be evaluated. The qMSP assay is performed at the time points listed above for the BI-PAP-A assay and can be run from the same sample. Results are analyzed against clinical outcomes and other results from immune and gene expression studies.

Doc2b as a potential biomarker of beta cell function. Blood drawn from islet transplant recipients is monitored for the presence of Doc2b protein before and after transplant. Doc2b is a ubiquitously expressed soluble 45 kDa protein that serves as a scaffold for SNARE regulatory exocytosis proteins near the plasma membrane. SNARE proteins ‘pin’ insulin granules to the cell surface to promote insulin release from the beta cell. A primary rate-limiting feature of beta cell function is the abundance of exocytosis proteins per beta cell; deficiencies in exocytosis proteins are considered an underlying cause of beta cell dysfunction. Doc2b is known to have an essential role in the beta cells, although it is expressed in multiple cell types. Pilot studies have demonstrated a significant association between attenuated Doc2b levels in pre-T1D NOD mouse platelets and the islets, supporting the concept that attenuated Doc2b levels in beta cells may be ‘reported’ by blood-borne platelets and could be useful as a potential biomarker for early detection of T1D and/or changes in functional beta cell status. Samples are drawn at least once pre-transplant, at Month 1 (Day +30+5) and Month 6 (Day +180±14) post start of EACH GAST-17 Treatment Course, and if/when islet graft dysfunction/rejection is suspected. A role for Doc2b as peripheral marker of beta cell function is supported by initial reported observations in islet transplant recipients that showed that Doc2b is deficient in insulin-dependent patients prior to transplant, but becomes detectable in the peripheral circulation post transplant (107).

### Potential Limitations

Since direct means of quantifying beta cell mass following intraportal islet transplantation are not yet clinically available/possible, mechanisms of GAST-17 effects post islet transplant are estimated indirectly based on metabolic outcomes. Evidence suggests a role for gastrin in beta cell expansion/neogenesis, which may increase the insulin secretory capacity of the islet graft and enhance transplant outcomes. It is also possible that gastrin augments beta cell function through other mechanisms. The instant statistical plan describes how data is analyzed to help evaluate for these effects.

### Statistical Plan, Data Analysis and Endpoints

Sample size calculation: 20 individuals are enrolled and followed for one-year after islet transplantation. Using data available through CITR on 347 islet transplant recipients, FDA investigators suggested a model for calculating sample size for a single arm islet transplant study using a range of hypothetical control rates of treatment effectiveness and corresponding number of subjects required to show superiority of islet transplantation using a composite endpoint that include freedom from severe hypoglycemia, insulin independence and HbA1c<6.5% in a single arm study (power=80% and alpha=0.05, one sided) (108). FDA recommendations were extended to include greater hypothetical reference treatment control rates to represent outcomes of a single islet transplant and the expected rate improvements of islet transplant with gastrin treatment and generated the corresponding required sample sizes for the current single arm islet transplant study. Data were provided by CITR on 125 subjects who were recipients of a single islet transplant using T-cell depleting immune suppression and TNF-alpha inhibitor at induction, a similar regimen to that proposed in the present protocol [CITR data in Insulin Independence and Composite Endpoint at Pre-Transplant, Day 75, and 1, 2, and 5-years post FIRST infusion data export]. This CITR data showed that 30 of 59 subjects who were evaluated at 1-year post transplant were insulin independent (50.8%). However, when the FDA-suggested composite endpoint (freedom of severe hypoglycemia, A1c <6.5%, and insulin independence) was assessed; only 10 out of 40 evaluated subjects satisfied this criterion (25%). Therefore, if the proposed T-cell depleting immune suppression induction regimen without gastrin use can achieve a composite response rate of 25% at one year (similar to the existing CITR data), and the gastrin regimen is able to achieve a 55% success rate (30% higher than the no gastrin-containing regimen), then a sample size of 17 subjects would be needed, per the extended FDA recommendations. However, in order to accommodate the possibility of early withdrawals, or a slightly lesser gastrin effect, this single-arm trial targets accrual of 20 subjects.

### Endpoints:

Safety endpoint: The safety of islet transplantation and GAST-17 treatment is evaluated by monitoring and summarizing adverse events throughout study follow-up. Key adverse events associated with IT, immunosuppression and gastrin and incidence of change or early discontinuation of gastrin treatment is summarized in regular reports to the Islet Cell DSMB. The IC-DSMB may place subject accrual on hold based on the following safety stopping criteria:

One Subject experiences grade 4 toxicity, where the toxicity or adverse event is serious and at least possibly related to GAST-17.

Two out of any three consecutive subjects experience grade 3 toxicities, where the toxicity or adverse event is serious, unexpected, and at least possibly related to GAST-17.

The primary efficacy endpoint is the proportion of subjects achieving a composite endpoint of insulin independence, freedom from severe hypoglycemia and HbA1c <6.5% (“complete response”) at 1 year post transplant/6 months post start of GAST-17 course II, in line with what has been previously suggested by the FDA (108) and compared to hypothetical controls, the data from a comparable protocol without the use of GAST-17, as well as to international data reported to the Collaborative Islet Transplant Registry (CITR) among islet transplant recipients who received a single islet transplant using a similar T-cell depleting immunosuppressive induction regimen without GAST-17 treatment.

Secondary efficacy endpoints: As concluded by the multi-center, Phase III CIT trial (14), islet transplantation may provide benefits even when insulin independence is not achieved (e.g. elimination of severe hypoglycemia and stabilization of glucose as reflected by HbA1c). Therefore, the following secondary endpoints are assessed at Month 1, Month 2.5 and Month 6 post start of each GAST-17 course:

Proportion of subjects who are free of severe hypoglycemic episodes (SHE) and have an HbA1c <7.0% (“partial response”). This is the primary efficacy endpoint used to define islet transplant success under the CIT-07, Phase III, multicenter islet transplant alone trial (14). Analysis of this endpoint allows to compare rates of success achieved under this trial with those published for the CIT study.

### Reduction/Elimination of Hypoglycemia

### Reduction in Daily Insulin Use

### Reduction of Daily Insulin Use Per 100,000 IEQ Transplanted

C-peptide/insulin secretion response to glucose potentiated arginine stimulation and other metabolic studies post the start of each 30-day GAST-17 course. This testing allows for evaluation of cumulative effects of the two 30-day courses of GAST-17.

Monitoring for efficacy: The proportion of subjects meeting the primary and secondary efficacy endpoints (defined above) is analyzed by the Kaplan-Meier method, with confidence bounds, and also as specified below following each treatment course. In addition, measures of allo- and autoimmunity and other biomarkers and their use as time-dependent variables possibly predicting changes in primary and secondary endpoints are assessed. Quality of life (QOL) measures before and after treatment are analyzed and compared by standard paired and longitudinal data methods.

Outcome measures and statistical analyses of Treatment Course 1. Preliminary outcomes of islet transplantation with GAST-17 treatment are assessed at Month 1, Month 2.5 and Month 6 post-islet transplant and initiation of the first course of GAST-17. During this early period of engraftment, it is not be possible to clearly distinguish the effects of the first course of GAST-17 from the functional improvements induced by the islet graft itself. However, a preliminary assessment of the effects GAST-17 on islet transplant outcomes is coarsely examined by comparing transplant efficacy outcomes from this trial to those reported internationally to CITR among islet recipients treated with a similar immunosuppressive induction regimen without GAST-17.

Outcome measures and statistical analyses of Treatment Course 2. Subjects receive a second 30-day course of GAST-17 after completing the Course 1, starting after month 6 visit. Critical assessment of GAST-17 effects are done by comparing insulin secretion in response to glucose-potentiated arginine stimulation, oral glucose tolerance testing (OGTT) and glucagon stimulation before and after the second GAST-17 course. The durability of GAST-17 effects are assessed by comparing the functional results at Month 1 and 2.5-month post initiation of GAST-17 Course 2 with that of Month 6 post initiation of the same GAST-17 course.

Monitoring for futility. Futility is assessed by tracking the number of subjects who achieve and maintain the primary complete response (insulin independent, hypoglycemia free, AND with HbA1c <6.5%) or partial response (SHE-free and HbA1c <7.0%) at 1 year post last transplant. Subjects are counted for this purpose when either of the following occurs:

1) The subject meets the complete or partial response definitions with adequate duration, or 2) The subject has failed to meet the complete or partial response definitions.

Monitoring for adequate efficacy is based on the data from CITR that 25% of single islet transplant recipients not receiving gastrin treatment achieved the primary endpoint [CITR, unpublished data, Insulin Independence and Composite Endpoint at Pre-Transplant, Day 75, and 1, 2, and 5-years post FIRST infusion data export, Jun. 20, 2016]. Monitoring is used to assure that the underlying one-year rate of insulin independence, with freedom from hypoglycemia and HbA1c <6.5%, is at least 25%. The study is stopped for futility if rate of meeting the composite endpoint at 1 year falls below 25% of treated participants.

Monitoring for quality. The incidence of primary islet graft failure, defined as negative C-peptide or no change from baseline (pre-transplant) daily insulin requirements within 30 days (±7 days) after transplant, is monitored to ensure that the islet isolation and transplantation process remains stable. Any single incidence of primary graft failure triggers the study team to investigate informally. Any three primary graft failure events within a sequence of 6 consecutive transplants is considered a formal alarm, requiring temporary closure, and a decision by the Islet Cell DSMB.

Other analyses: In addition, measures of allo- and autoimmunity and other biomarkers and their use as time-dependent variables possibly predicting changes in primary and secondary endpoints is assessed. Changes in quality of life (QOL) before and after treatment involves summary of changes in scores from the QOL instruments by standard paired and longitudinal data methods.

### Example 3: Gast-17 Improves Insulin Secretion and Sensitivity in Individuals with Type II Diabetes

Gastrin is expressed in the insulin+ and somatostatin+ islet cells of people with T2D, likely to promote beta cell recovery and expansion. It was shown that gastrin promotes beta cell proliferation and possibly differentiation of pancreatic ductal cells into insulin+ cells. It was found that human islets from elevated HbA1c donors treated with gastrin showed increased expression of islet hormones (insulin, glucagon, somatostatin) and beta cell transcription factors (PDX1, MNX1, SMAD9, HHEX, MAFA, SOX5). Also, gastrin stimulated the transformation of delta cells into insulin+/somatostatin+ cells, with increased insulin gene expression correlating positively with donor HbA1c level. Pilot data also showed that long-term islet exposure to gastrin increased expression of NGN3, nestin, urocortin3, PPY, and MAFB, and increased cell proliferation and numbers of insulin+/somatostatin+ cells, while reducing inflammatory gene expression. Gastrin also protected islets from inflammatory cytokines and increased their insulin production to glucose. Thus, gastrin is a promising islet hormone secretagogue, an inhibitor of islet inflammation, and a promotor of cell growth/trans-differentiation. Moreover, the beneficial effects are most evident in individuals with elevated HbA1c who have more beta cell dysfunction (FIG. 1).

A clinical grade gastrin analogue (GAST-17) was manufactured with FDA approval for an ongoing clinical trial evaluating its use to improve islet function in type 1 diabetic islet transplant recipients. Initial results are promising, with the first two individuals treated with GAST-17 and a single islet transplant achieving insulin independence with half of the islet mass normally required. These data inform the current hypothesis that GAST-17 promotes beta cell differentiation/neogenesis, and insulin secretion, while reducing islet and systemic inflammation to improve insulin secretion and sensitivity in individuals with T2D. To test this hypothesis, state-of-the-art PET/MRI technology was use with a novel PET tracer, [⁶⁸Ga]-DO3A-VS-Cys40 Exendin-4, to image native pancreatic islets, and a PET ¹⁸fluorodeoxyglucose (FDG or ¹⁸F-glucose) tracer to image whole body insulin sensitivity responses to GAST-17. Results of these imaging studies are correlated with advanced metabolic testing and immune profiling. The hypothesis is tested with 3 aims:
1) To establish the safety, tolerability and dosing of GAST-17 treatment in patients with T2D. This is achieved through obtaining all regulatory approvals. The clinical trial includes two treatment phases; a GAST-17 dose escalation phase and a randomivzed GAST-17 treatment versus standard of care phase.
2) To assess the therapeutic efficacy of GAST-17 treatment at 3, 6 and 12 months of follow up by determining metabolic responsiveness as compared to standard of care controls (HbA1c, antidiabetic drug use, and in GAST-17 treated subjects compared to pretreatment (maximum insulin secretion in response to hyperglycemic clamp followed by arginine stimulation, and models of insulin sensitivity).
3) To characterize GAST-17 mechanisms of action, including: a) biomarkers of inflammation and their relationship to changes in insulin secretion and sensitivity as measured by metabolic parameters and imaging technologies, and b) functional beta cell mass and whole body insulin resistance imaging using novel PET/MRI technologies and the new [⁶⁸Ga]DO3A-VS-Cys40 Exendin-4 and a standard ¹⁸F-glucose PET probes.

Results described herein establish GAST-17 as the first T2D pathophysiologic-directed therapy to improve glycemic control, resolve inflammation AND promote beta cell function/expansion. It also advances islet/metabolic imaging technologies critically needed in the diabetes field for direct monitoring of pancreatic islet mass and whole-body insulin sensitivity.

### Improvements and Innovation

Inflammation is important in the pathophysiology of T2D. In contrast to certain anti-inflammatory strategies, gastrin broadly inhibits expression of multiple inflammatory genes and cytokine production by islets, in addition to promoting expansion/proliferation of pancreatic beta cells, as well as favoring M2 over M1 macrophages. Thus, gastrin is better positioned to resolve T2D-associated islet inflammation, beta cell dysfunction, in addition to potentially reducing systemic inflammation, and consequently improving insulin sensitivity.

Mechanistic studies evaluating gastrin effects on inflammatory cells, cytokine levels and circulating extracellular vesicle (EVs) may yield novel insight into the pathophysiology of T2D, as well as gastrin treatment effects. Studies performed before and after gastrin-treatment on monocyte-induced macrophage polarization into M1 and M2 phenotypes and their transcriptional signatures, and studies on circulating blood EVs from treated patient and normal non-diabetic control on monocyte-derived macrophage polarization, and on healthy non-diabetic human islet function in vitro, provide additional valuable pathophysiologic information.

State-of-the-art PET/MRI is used to non-invasively visualize human beta cell expansion and whole-body insulin resistance in people and correlate imaging data with advanced biochemical metabolic parameters. This brings on-line safe methods for tracking islet survival, proliferation and function as well as changes in insulin resistance, and can be applied to assessing the effects of a variety of new drugs in development.

A wide array of therapeutic agents for T2D are available but none simultaneously target islet inflammation and beta cell expansion/neogenesis. Most drugs ignore the ongoing inflammation and diminished islet beta cell mass. Even GLP-1, another gut hormone, and its analogues, do not expand beta cells at clinically approved doses.

Further described herein are methods for moving the field of T2D therapy forward. The instant results also have implications for individuals with T1D, where islet inflammation is the major part of the pathophysiology. For example, Applicant's studies address: the maximum safe dose of the gastrin analogue GAST-17; whether GAST-17 increase islet beta cell mass or merely improve beta cell function; whether GAST-17 suppression of T2D-associated islet inflammation reflected in changes in macrophages, T-cells or circulating cytokine profiles; if GAST-17 reduce the adverse effects of T2D EVs on beta cell function; how long after gastrin therapy do effects upon islets, inflammation, and beta cell growth last; and whether PET/MRI imaging is sensitive enough to show gastrin-induced changes in beta cell mass.

None of the existing diabetes medications, including GLP-1 analogues, address islet inflammation or can truly expand beta cell mass at approved clinical doses. In contrast, gastrin at the current clinically tested doses can limit islet inflammation and can induce beta cell expansion/neogenesis and enhance beta cell functional capacity.

Standard anti-glycemic agents do not directly reduceT2D islet inflammation and injury and therefore cannot reverse disease. The instant preliminary studies showed that gastrin can promote beta cell proliferation and function and limit inflammation, leading potentially to reversal of islet dysfunction of T2D.

### Data

Gastrin prevents in vitro death of human islets. Extended cell culture promotes islet death. Human islets from individuals without diabetes were treated with gastrin (100 nM). After 2 weeks, islets were incubated with propidium iodide (PI) to stain dead cells. Interestingly, gastrin treated islets showed fewer PI+ cells (FIG. 17, lower image) compared to control islets (FIG. 17, upper image). Thus, treatment with exogenous gastrin is sufficient to enhance islet health during extended culture.

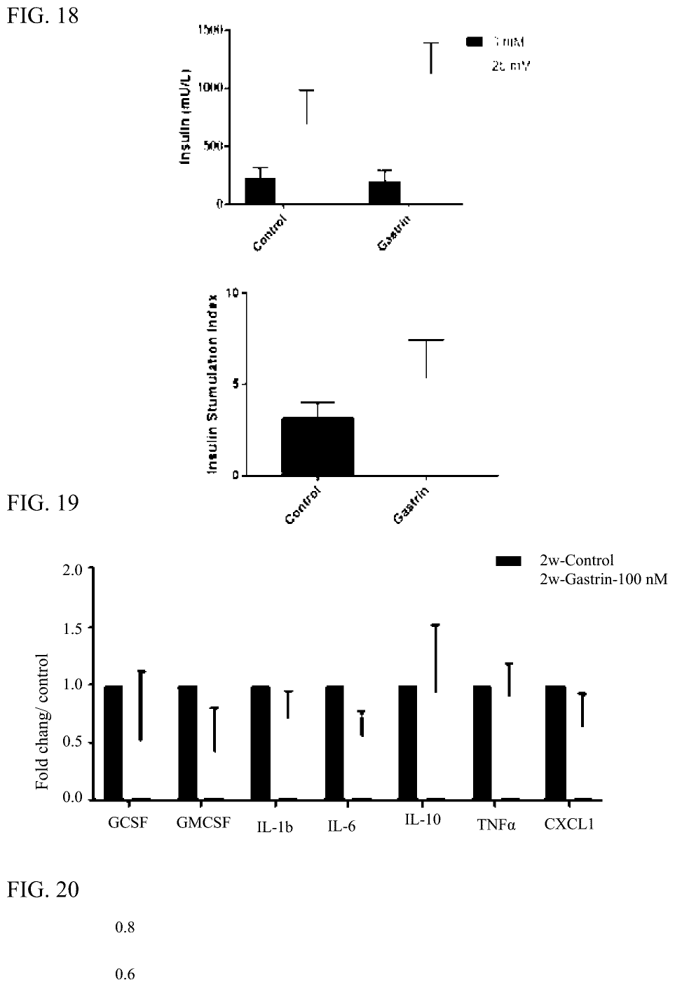

Gastrin improves human islet function. While enhanced survival was seen in islets cultured with gastrin at 2 weeks, it is possible this did not translate into functional responses. To test this, human islets from individuals without diabetes (500 IEQ) were cultured in standard islet medium ±exogenous gastrin (100 nM) for 2 weeks and then challenged the islets with glucose (25 mmol/l). Both, control, and gastrin-treated islets showed increased insulin release (FIG. 18, upper graph). However, the gastrin-treated islets showed a higher insulin stimulation index (calculated by insulin concentration in response to high glucose divided by that to low glucose) compared to the control islets) (FIG. 18, lower graph).

In islets, culture-related induction of inflammatory genes was decreased by gastrin. T2D is characterized by chronic inflammation in general and islet inflammation in specific and this contributes to dysregulation of glucose metabolism. Immune cells and islets secrete inflammatory cytokines (33). Human islets were cultured for 2 weeks in standard media ±gastrin and changes in mRNA levels determined. Gastrin-treated islets displayed decreased mRNA levels of multiple pro-inflammatory genes compared to untreated islets (FIG. 19), especially in islets from individuals with a history of poor glycemic control.

Gastrin limits soluble cytokines secretion by cultured islets. While lower transcript levels of inflammatory cytokines suggest less signaling, they may not parallel soluble cytokine levels. Human islets were cultured for 2 weeks ±gastrin and protein levels of inflammatory cytokines determined in the conditioned medium. Secreted cytokine IL-1 levels were markedly less in medium from islets treated with gastrin (FIG. 20). In treated islet condition medium multiple cytokines (interleukins 4, 6, 7, 8, 10 and 21) were also decreased (data not shown). Thus, exogenous gastrin suppresses inflammatory cytokines at the gene and protein level.

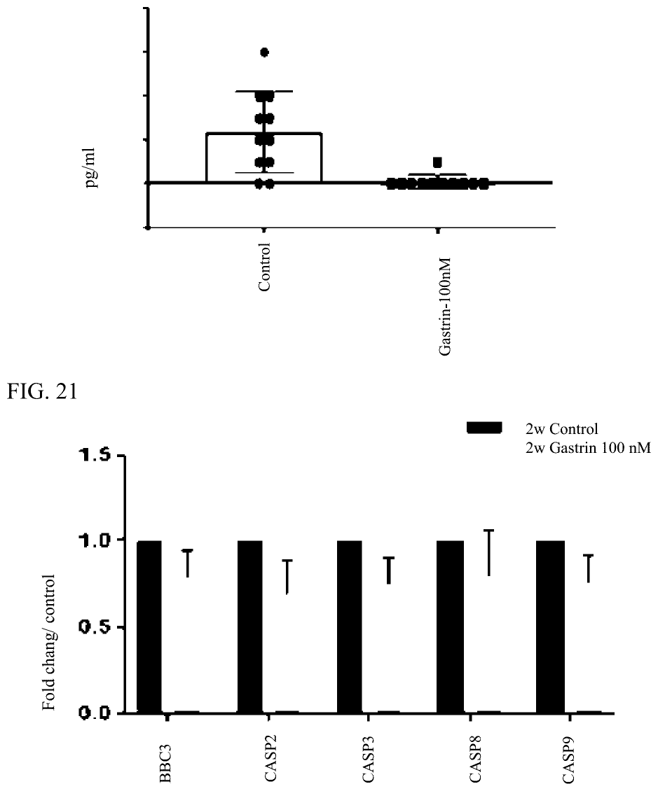

Long-termed cultured human islets treated with gastrin showed lowered mRNA levels of several apoptotic genes. In non-cancer cells, gastrin deceased apoptosis (34). Similarly, in long-term cultured islets, gastrin treatment decreased cell death and mRNA levels of genes that promote apoptosis (FIG. 21).

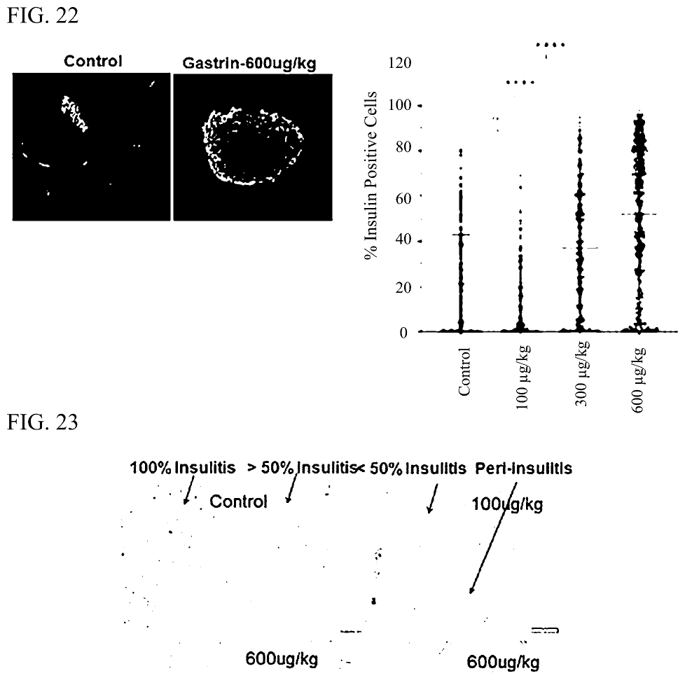

Exogenous gastrin promotes human beta cell expansion. The above data indicated that human islets damaged by long-term culture could be salvaged, inflammation decreased, and function improved by gastrin treatment. Particularly encouraging was the finding that the more severe the injury environment that the islets were exposed to (as signified by higher HbA1c levels in the organ donor) the more effective gastrin was. T2D is characterized by a loss of beta cell mass (35). NOD mice, that develop insulitis and beta cell death, were treated with various doses of gastrin (100, 300 [equivalent to lowest suggested clinical dose] and 600 μg/kg) and assessed for insulin positive cells. Interestingly, animals given gastrin showed increased numbers of insulin+ cells in a dose-dependent manner (FIG. 22). Thus, in an inflammatory microenvironment, gastrin limits loss of insulin+ cells.

Exogenous gastrin deceases insulitis in diabetic rodents. Insulitis is defined as invasion of inflammatory cells into the pancreatic islets. Diabetes, both type 2 (36) and 1, are characterized by islet inflammation, termed insulitis. Rodents known to develop hyperglycemia and insulitis (NOD mice) were given gastrin and the amount of islet immune cell invasion characterized. As noted, the control animals displayed increased inflammatory cell invasion in pancreatic islets and this was less in the islets from animals treated with gastrin (FIG. 23). These data support the hypothesis that exogenous gastrin is both an immune cell suppressant and expands beta cell mass.

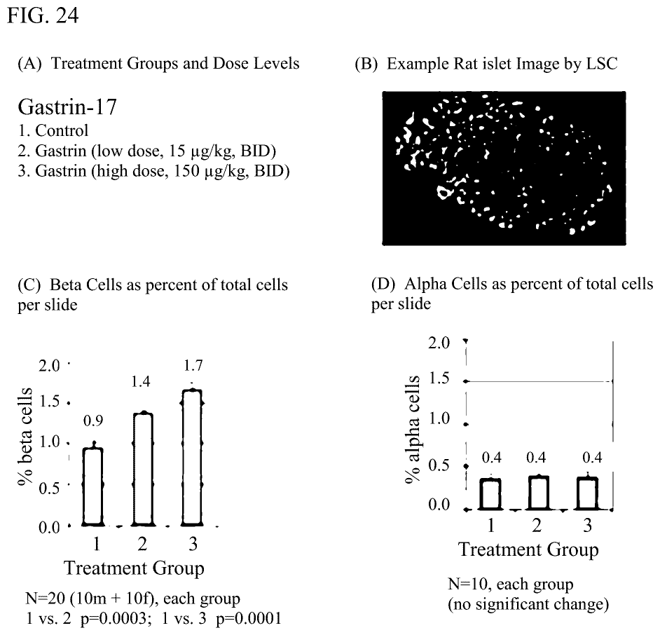

Gastrin analogue, GAST-17, stimulates beta cell expansion. T2D individuals display a loss of beta cell numbers and function. Increasing beta cell mass is considered a possible therapy for T2D (37). The islet expansion effects of GAST-17 were evaluated in non-diabetic Wistar rats (10 males and 10 females in each group). At the end of 30-day treatment, pancreata were excised and stained for beta and alpha cell content counting using laser scanning cytometry (FIGS. 24A and 24B). The average percentage of beta cells of total cells per slide significantly increased after gastrin treatment, while the percentage of alpha cells did not change (FIGS. 24C and 24D, below).

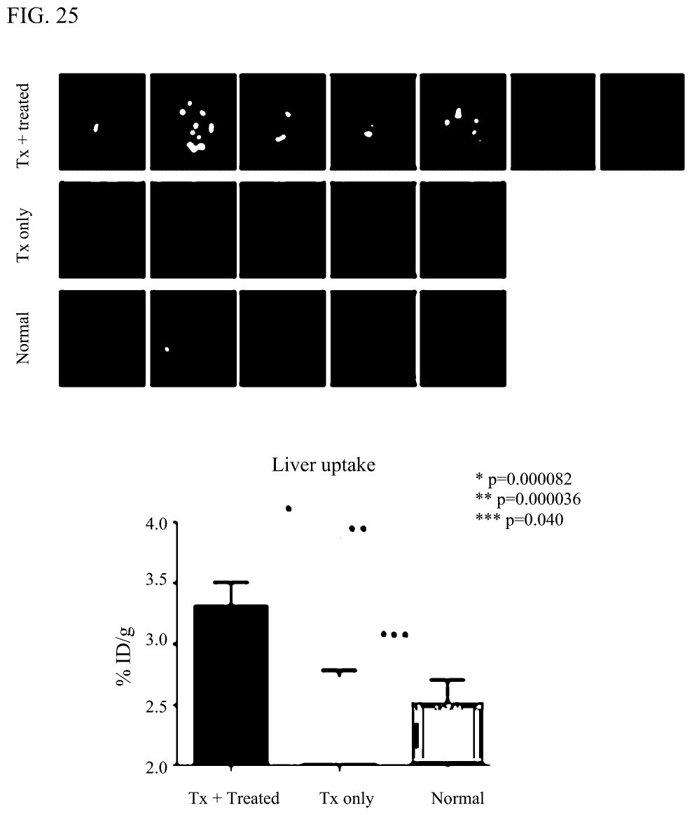

Gastrin analogue, GATS-17, promotes expansion/neogenesis of transplanted human islets. Isolated human islets were transplanted (Tx) into livers of NOD/SCID mice followed by GAST-17 treatment for 30 days (150 μg/kg/dose, injected three times daily) (Tx+Treated, n=7) and compared to mice receiving islet transplant alone (Tx only, n=5) and untreated controls (Normal, n=5). After completion of treatment, whole mice and organs of interest were imaged (in vivo and ex vivo) with ¹⁸F-TC-Exendin-4 (TCE4) using microPET (a high specific activity labeling technique developed at COH for targeting islets). Compared with the control group, uptake by islet grafts in liver of the GAST-17-treated group were significantly higher by both in vivo and in excised livers ex vivo imaging (FIG. 25).

Type 1 diabetics treated with GAST-17 and islet transplant reversed diabetes with smaller islet mass and had no treatment-related adverse side effects. Poorly controlled T1D individuals with severe hypoglycemia can be rescued with islet transplantation (IT) to the liver which restores normoglycemia. However, such results require a large number of islets be given (usually more than one transplant). IT imparts a severe ischemic and inflammatory stress on islets, and many islets do not survive the process. The safety and islet-protective properties of gastrin were tested. Two T1D individuals underwent (IT) and followed with two courses, one month each (month 1 and 7) of gastrin therapy (15 μg/kg twice daily), They showed rapid engraftment and total normalization of blood glucose with near half the usual number of islets (<6,100 as compared to >10,000 IEQ/kg). (FIG. 26). As well, both individuals reported no adverse effects related to gastrin.

Taken together, these data provide evidence that gastrin, and the gastrin analogue GAST-17, are anti-inflammatory, anti-apoptotic and pro-growth for human and rodent islets. As well, GAST-17 expanded islets in the native pancreata of animals and human islets (FIGS. 22, 24 and 25).

GLP-1R is an islet-specific cell membrane protein and the target for a novel islet radiolabel probe. An ideal imaging probe should be highly specific to the intended target. GLP-1R is restricted in its expression and is the target of Exenden-4 and of the current probe. NOD SCID mice received, via the portal vein, 1000 human islet equivalents. Livers were harvested 12 days post-transplantation. Immuno-fluorescent staining showed no significant difference in GLP-1R expression between the islets transplanted to the liver and native islets in the human pancreas (FIG. 27). The presence of GLP-1R immunostaining in the livers (ordinarily GLP-1R-negative) is consistent with engraftment of islets. Staining for insulin confirmed the presence of human islets. Insulin+ cells are also GLP-1R+ in islets engrafted in mouse livers. These data show that GLP-1R is confined to islets.

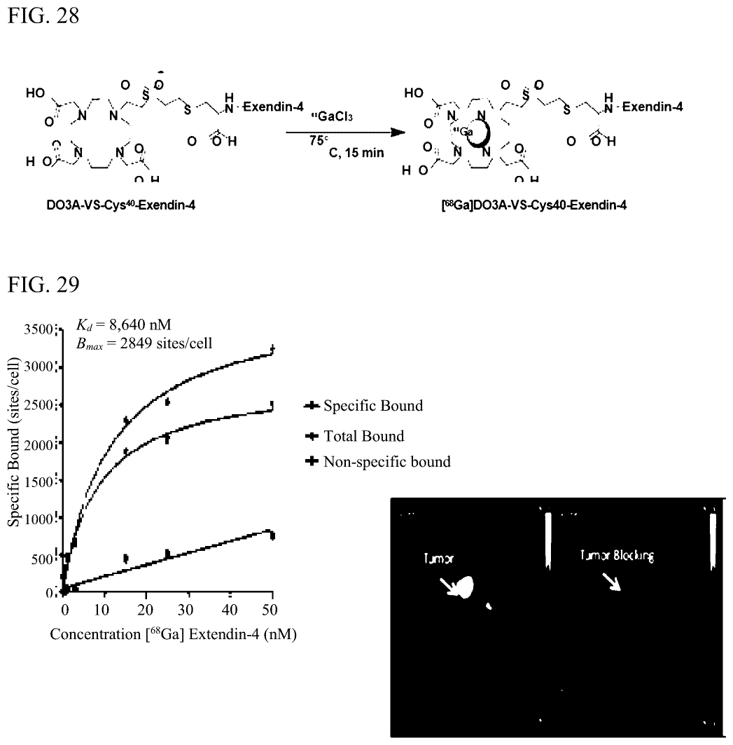

The islet-specific radiolabel [⁶⁸Ga]-DO3A-VS-Cys40-labeled Exendin-4 can be synthesized under cGMP conditions. ⁶⁸Ga was obtained from a bench-top ⁶⁸Ge/⁶⁸Ga generator system (1850 MBq, Eckert & Ziegler, IGG 100), and eluted with 0.1 M HCl. The first 1.5 mL fraction was discarded and the next 3.0 mL fraction was collected in a glass vial containing 10.5 nmol DO3A-Exendin-4 buffered with 2 M sodium acetate and radical scavengers. The mixture was incubated at 75° C. for 15 minutes. The final product showed high radiochemical purity (95%) (FIG. 28) [0348] [⁶⁸Ga]-DO3A-Exendin-4 has a high binding affinity for, and specificity to, GLP-1R. Saturable [⁶⁸Ga]-DO3A-Exendin-4 binding was observed in INS-1 cells, that express GLP-1R, with a Kd of 8.60 nM (FIG. 29, left graph). The radiolabel had a high binding affinity for INS-1 cells in vitro and in vivo. Biodistribution and microPET imaging of [⁶⁸Ga]-tracer were performed on mice with INS-1 tumors. Data demonstrated that [⁶⁸Ga]-DO3A-Exendin-4 can specifically bind to GLP-1R in vivo (FIG. 29, radiographs). The binding of the radiolabeled probe was significantly reduced in the presence of excess unlabeled Exendin-4. The probe had much lower liver uptake (˜2% ID/gram 90 minutes post-injection) indicating that probe uptake by the liver does not confound islet detection.

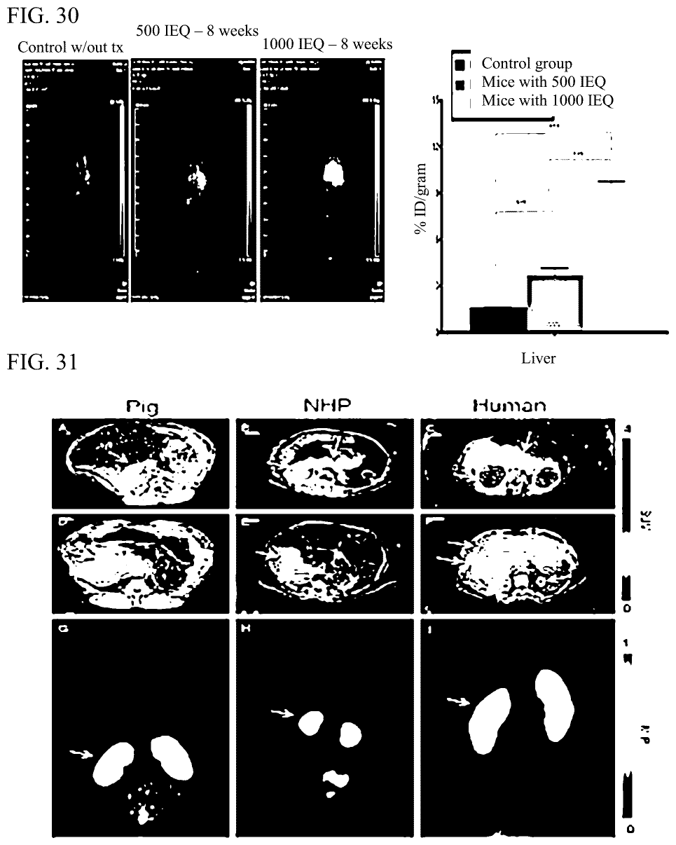

Human pancreatic islets are imaged with PET. T2D and T1D science remains stymied by a lack of non-invasive methods to detect pancreatic islet changes after clinical interventions and in relation to disease progression. Thus, conclusions on treatment effectiveness are based upon indirect tests such as blood glucose levels and markers of hyperglycemia like HbA1c. To date, correlation with real-time pancreatic islet mass and function has not been possible. Human islets (500 and 1000 IEQ) were transplanted into NOD SCID mice via portal vein injection. MicroPET scanning was performed with [⁶⁸Ga]-DO3A-Exendin-4 at 8 weeks post-transplantation (FIG. 30, below). Mice with 1000 IEQ had significantly higher probe uptake, demonstrating GLP-1R-enriched islets in the liver. Uptake values for the liver were 1.60±0.02% ID/g for the controls, and 3.67±0.46 and 9.36±0.39% ID/g for mice given 500 IEQ and 1000 IEQ (FIG. 30, below). The hepatic uptake of tracer in mice that received 1000 IEQ was 6-fold higher than controls, confirming the probe targets GLP-1R-positive transplanted human islets.

[⁶⁸Ga]-DO3A-Exendin-4-PET imaging is safe and specific in pigs, non-human primates and one patient with malignant insulinomas. [⁶⁸Ga]-DO3A-Exendin-4 was employed to image insulin-producing islets in pigs, non-human primates, and in one patient with an insulinoma. A high degree of contrast between normal pancreatic islet uptake and metastatic insulinoma, compared to hepatic uptake, was achieved. Insulinoma metastases in the patient's liver were clearly visible (FIG. 31, below). The kidney dose was 0.34±0.06 (rats), 0.28±0.05 (pigs), 0.65±0.1 (non-human primates), and 0.28 mGy/MBq (human). The estimated maximum dose that can be administered annually to human is 150 mGy. Thus, these data indicate that [⁶⁸Ga]-DO3A-Exendin-4 can be safely administered repeatedly for PET imaging studies of pancreatic islets in humans.

Gastrin protects against myocardial ischemia reperfusion injury (IRI). A recent study in rats showed gastrin improved myocardial function and reduce myocardial injury markers, infarct size, and cardiomyocyte apoptosis induced by IRI (38). Gastrin increased the phosphorylation levels of ERK1/2, AKT, and STAT3 indicating its ability to activate the RISK (reperfusion injury salvage kinase) and SAFE (survivor activating factor enhancement) pathways. Inhibitors of ERK1/2, AKT, or STAT3 abrogated the gastrin-mediated cardiac protection.

Data Summary in Relation to Clinical Trial Design. The instant preliminary studies, together with others (38), suggest that gastrin and GAST-17 treatment of non-diabetic animals induces beta cell expansion/neogenesis. The preliminary in vitro data with human islets from diabetic/prediabetic donors shows that this effect may depend on overall glycemic control and involve reprogramming of pancreatic and islet cells. Gastrin may also have protective effects in other situations of inflammation such as cardiac IRI.

Thus, Applicants determined whether gastrin analogue GAST-17 promotes beta cell differentiation/neogenesis, and insulin secretion while reducing islet and systemic inflammation resulting in improved insulin secretion and sensitivity in individuals with T2D, and therefore, represents a first-in-class, pathophysiology-targeting, beta cell mass recovery and protective agent.

### Research Design and Methods

Objectives of the studies are described below.
1) Determine the safety of GAST-17 therapy in TD2 recipients. Safety is evaluated by monitoring adverse events over a one-year follow-up period.
2) Determine if GAST-17 treatment improves T2D outcomes, through assessing if the primary efficacy endpoint is improved glycemic control as reflected by a reduction of HbA1c by >1% at the end of gastrin treatment course (12 weeks). A secondary endpoint is a reduction in daily diabetic medication use by >25% at 6 months from the beginning of 12 weeks GAST-17 therapy, without adding new anti-hyperglycemic therapeutic agents or new behavior modification interventions. Another efficacy endpoint is if GAST-17 exerts effects on beta cell expansion/neogenesis and/or enhances beta cell functional capacity in T2D individuals. Clinical methods for measuring beta cell mass in people directly are not available. Beta cell expansion/neogenesis and durability of GAST-17 effects is evaluated by comparing beta cell functional responses (C-peptide/insulin secretion in response to metabolic stimulation such as hyperglycemic glucose clamp/arginine infusion) before and at 3, 6 and 12 months after the start of GAST-17 treatment, and by measuring circulating levels of Doc2b, a novel biomarker of beta cell function, as compared to standard of care controls. The hyperglycemic glucose clamp/arginine test is done only twice in controls, at baseline and 6 months. Also, changes in insulin sensitivity can be determined using models of insulin sensitivity and whole-body ¹⁸F-glucose uptake imaging in both groups (see below).
3) Perform mechanistic studies to characterize GAST-17 actions including: a) effects on markers of inflammation including cellular (immune/inflammatory), circulating biomarker (EVs), and molecular (cytokines), and their relationship to changes in insulin secretion and sensitivity as measured by metabolic parameters and imaging technologies, and b) changes in novel PET/MRI imaging parameters using a newly developed [⁶⁸Ga] DO3A-VS-Cys40 Exendin-4 and a standard ¹⁸F-glucose PET probe to image GAST-17-mediated changes in the native pancreas islet mass and body insulin resistance, and to correlate image parameters with metabolic testing results and changes in inflammatory cell and cytokine profiles.

### Study Design

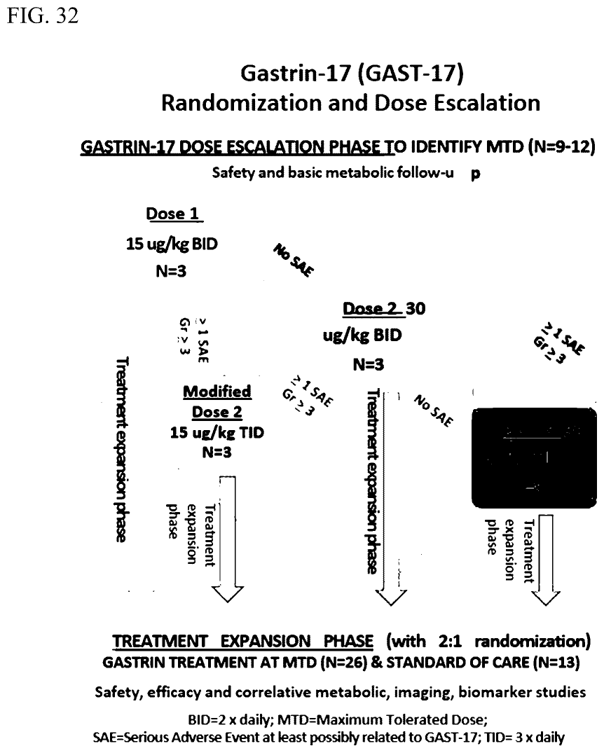

This is a Phase I/Ib, prospective, single arm, single site trial to assess the safety and efficacy of GAST-17 in T2D subjects. The Dose Escalation Phase is to determine the MTD of GAST-17. The Treatment Expansion Phase expands the number of subjects within the MTD cohort by adding an additional 26 subjects to evaluate safety and efficacy of GAST-17 during a one-year of post-treatment follow-up (FIG. 32), in addition to 13 controls followed up by standard of care measures. This trial establishes the safety and efficacy of gastrin treatment to enhance the insulin producing capacity of native islets, and thereby induce metabolic stability, achieve glucose homeostasis and restore beta cell function while suppressing T2D-associated inflammation. Detailed metabolic studies characterize islet functional changes and proposed immunologic and biomarker studies chart the interplay between immune/inflammatory and other mechanisms contributing to islet dysfunction in T2D. Controls are evaluated by all described parameters at all study time-points except as indicated in the relevant section below.

### Dose Escalation Phase

GAST-17 is administered at different doses to evaluate its safety. To this end, 3 dose levels set at 15 μg/kg BID, 30 μg/kg BID, and 30 μg/kg TID are explored. (Table 2). The 3 patients studied at the highest possible dose with no SAE is considered the MTD.

TABLE 2
GAST-17 Dose Escalation

| Dose Level | Dose |
|---|---|
| Dose Level 1 | 15 μg/kg Twice Daily (BID) |
| Dose Level 2 | 30 μg/kg Twice Daily (BID) |
| Modified Dose Level 2 | 15 μg/kg Three Times Daily (TID) |
| Dose Level 3 | 30 μg/kg Three Times Daily (TID) |

### Treatment Expansion Phase

The Treatment Expansion Phase uses 2:1 randomization to the GAST-17 versus standard of care (26 new GAST-17 treated subjects at MTD and 13 standard of care subjects). These 39 randomized subjects are monitored over one year for safety, efficacy and correlative studies. Expansion subjects who drop out before Month 6 are considered unevalauble and replaced.

### Study Population

Up to 57 T2D adults (age 18-70 yrs), who are not on insulin, GLP-1 agonist, DPP-4i, Symlin treatment and have HbA1c of 7 to 9.5% and no exclusion factors participate in the study. These include up to 12 subjects treated with gastrin during the Dose Escalation and 26 gastrin treated subjects during the Treatment Expansion Phase (26), together with 13 comparable adults with T2D who do not receive gastrin therapy.

### Enrollment and Retention Plan

Initially, all accrued subjects are allocated to the dose escalation phase, but once the MTD has been determined, the Treatment Expansion Phase begins with randomization of subjects to Gastrin treatment or control arms at a ratio of 2:1 respectively, based on HbA1c and number of oral diabetes medications at enrollment.

### Safety Assessment of Gast-17

Subjects are monitored for adverse events related to GAST-17 treatment. Subjects continue to be assessed for safety in the outpatient clinic every 4 weeks during GAST-17 therapy, and at months 3, 6, 9 and 12 from the beginning of treatment. Outpatient visits include review of symptoms, vitals/weight/BMI, review of blood glucose logs, physical exam, lab assessments (CBC, biochemical, and other parameters), and assessment for changes in diabetes complications (urine protein excretion, neuropathy, retinopathy).

Adverse event collection. All adverse events reported or observed since the time of the last clinic visit are recorded and graded per the Common Terminology Criteria for Adverse Events Version 5 (CTCAE v 5.0). Safety stopping criteria are in place if Grade 3 or higher adverse events associated with gastrin therapy are observed (see Statistics, below).

### GAST-17 Treatment Efficacy on Glycemic Control

Efficacy is assessed in terms of glycemic control. HbA1c is measured before and at month 3, 6, 9, and 12 from the start of each GAST-17 treatment course to track improvements in glycemic control. The primary endpoint for assessing efficacy in the trial is the proportion of GAST-17-treated subjects achieving a reduction of HbA1c by >1%. A secondary efficacy endpoint is reduction in daily diabetic medication use by >25% at 6 months from the beginning of the 12-week GAST-17 therapy, without adding new anti-hyperglycemic therapeutic agents or new behavior modification interventions. For comparison of functional trends between those receiving GAST-17 treatment versus standard of care, T2D controls are evaluated by HbA1c measurement and all metabolic parameters at all time points except for the MSIS and the imaging studies which are done only twice at baseline and at 6 months.

### GAST-17 on Beta Cell Function and Insulin Resistance

Changes in beta cell function and insulin resistance induced by GAST-17 treatment is determined and compared with standard of care treated subjects, before and serially after 3, 6 and 12 months of the start of GAST-17 treatment. On each of these time points, body mass index (BMI), fasting plasma glucose, C-peptide, insulin and proinsulin, amylin and leptin levels are determined at two time points, 10 minutes apart, and the relevant parameters of these are used to estimate beta cell function using HOMA2-% beta and insulin sensitivity using HOMA2-% sensitivity. Both calculations are driven from the online software of the Diabetes Trial Unit, Oxford, UK (Diabetes Trial Unit, HOMA calculator, Version 2.2.3, 2014). Hepatic insulin resistance is calculated according to the Matthews et al. formula, HOMA-IR (39). Insulin sensitivity is calculated using QUICKI, another surrogate index of insulin sensitivity that correlates well with glucose clamp results in human including T2D patients (40). GAST-17 treatment effects on beta cell mass/function are assessed using maximal stimulated insulin secretion (MSIS) during hyperglycemic glucose clamp with added arginine administration (control subjects have the MSIS done only twice at baseline and at 6 months). The study was used to monitor beta cell survival and functional beta cell mass in IT recipients (41). MSIS tests are performed on the day after imaging, with minor modifications. Briefly, after 20 minutes of acclimatization to the i.v. catheters, blood samples are taken. A hyperglycemic clamp is performed at time t=0, using a variable rate infusion of 20% glucose to achieve a plasma glucose concentration of approximately 340 mg/dl, which is maintained for 45 minutes, followed by i.v. administration of 5 mg of arginine. Blood samples are collected at 2, 3, 4, 5, and 30 minutes, centrifuged, and used for measurement of glucose and insulin. MSIS is correlated with PET results (SUV). Insulin resistance is evaluated with ¹⁸F-glucose whole body PET/MR, as described below.

### GAST-17 Effects on a Novel Circulating Islet Function Biomarker

Doc2b is a potential biomarker of beta cell function. Doc2b serves as a scaffold for SNARE regulatory exocytosis proteins near the plasma membrane to promote insulin release from beta cells. Deficiencies in exocytosis proteins are an underlying cause of beta cell dysfunction. Dr. Thurmond's group at COH have demonstrated a significant association between attenuated Doc2b levels in NOD mouse blood and the islets (unpublished data). These findings support the concept that attenuated Doc2b levels in beta cells may be ‘reported’ in circulating blood and could be useful as a biomarker of degraded islet capacity. Doc2b levels in circulating blood of study subjects are characterized before and after GAST-17 treatment.

### Mechanistic and Correlative Studies

Several state-of-the-art correlative studies are conducted to unravel and/or confirm the mechanistic aspects of GAST-17 actions.

### Inflammatory and Immune Profiles

Peripheral blood samples are drawn from control subjects and Gast-17 treated subjects at baseline prior to GAST-17 treatment and at 3, 6 and 12 months after the start of treatment to evaluate anti-inflammatory and immunologic effects of treatment. To compare immunologic trends between those receiving GAST-17 treatment versus standard of care, T2D controls are evaluated at enrollment and all time points listed above. To avoid handling variation, all samples are processed into PBMC and plasma fractions, freezer-stored and batch analyzed at the completion of the clinical trial. Immune markers associated with Th1 and Th2 phenotypes and inflammatory milieu are determined by fluorochrome technology (Luminex) including TNF-α, TGF-131, IL-113, IL-6, IL-10, IL-13, IL-17, IFN-7, DCS, XCL5/ENA78, CXCL6/GCP2, CXCL10/IP10, CXCL12/SDF1a CCL2/MCP1, CCL4, CCL5, CCL13/MCP4, CCL19/MIP3b, and sTNFR11 (42). These cytokines are monitored before starting GAST-17 treatment, and at the other time points specified above after initiation of the GAST-17 treatment course. Peripheral blood mononuclear cells (PBMCs) are analyzed by flow cytometry to track changes in immune cell populations before and after GAST-17 treatment. Composition (percent and absolute counts) of B-cell, monocyte, natural killer (NK) cell, and T-cell subsets are determined. In addition, since macrophages are tissue resident cells and not present in circulating blood in significant numbers, GAST-17 treated subjects, but not controls, leukapheresis is performed at pretreatment and at conclusion of GAST-17 treatment for assessment of monocyte-induced macrophages polarization into M1 and M2 phenotypes as well as their transcriptional signatures (43, 44). In addition, circulating blood EVs of gastrin-treated and control subjects are isolated before and at conclusion of treatment and their effects on patient monocyte-derived, and on normal non-diabetic control monocyte-derived macrophages (obtained from the COH blood bank) and on healthy non-diabetic human islets (from the COH human islet distribution program) is assessed.

### Functional Imaging of Beta Cell Mass and Insulin Sensitivity

Imaging of functional beta cell mass in native pancreas and insulin sensitivity using a novel PET/MRI technology and the newly developed ⁶⁸Ga-DO3A-VS-Cys40 Exendin-4 radiolabel and a standard ¹⁸F-glucose PET probe provides precise real-time evidence for the expansion of beta cell mass through enhanced uptake of ⁶⁸Ga-DO3A-VS-Cys40 Exendin-4 by the pancreatic islets, particularly at the 6 month's timepoint post-treatment when any effect of GAST-17 on islet function would have dissipated. Simultaneous use of MRI aids in improving image quality while allowing MRI imaging of pancreas and liver fat infiltration. In addition, ¹⁸F-glucose PET imaging provides a novel tool for illustrating treatment-induced changes in total body insulin sensitivity. These imaging studies are done in T2D subjects who are treated with the selected dose of GAST-17 for the Treatment Expansion Phase prior to treatment and at 3, 6 and 12 months of follow up. Control subjects are done only at baseline and 6 months since changes in parameters are not expected in this group.

PET imaging sequences acquisition and analysis. Prior to PET/MR imaging, blood glucose levels are controlled for at least 48 hours to avoid effects of hyperglycemia on GLP-1R expression. Patients fast 6-8 hours prior to the study. An MRI transmission scan is obtained first to identify the region of the pancreas. Then [⁶⁸Ga]-DO3A-Exendin-4 (1.35-2.70±10% mCi) is given i.v. and a 60-minute dynamic PET scan performed over the pancreas region, followed by three whole body scans at 70, 120 and 240 minutes after probe administration. Blood samples are drawn before and at 5, 30, and 60 minutes after probe infusion to determine metabolic stability and glucose levels. Vital signs are taken at 5, 10, 20, 30, 45, and 60 minutes after the initiation of the PET study, and a final set of vital signs and an ECG are repeated before discharge. A urine specimen for HPLC metabolite analysis is collected at the end of the whole-body scan. PET scan data are analyzed (including volumetric region of interest [ROI] analysis and extraction of tissue time activity course [TAC]s and steady-state standard uptake value [SUV]s) and quantitative analysis of plasma TACs and HPLC data used to determine TACs for circulating [⁶⁸Ga]-DO3A-Exendin-4 and its metabolites as a function of time and to calculate cumulative activities for normal organs/tissues. Estimated total administered radiation dose for 1 year equals 4.3-10.8 mCi (160-400 MBq) depending on study arm and age of the ⁶⁸Ga generator.

To evaluate the efficacy of GAST-17 treatment on insulin resistance, whole-body PET-MRI imaging is performed using ¹⁸-fluorodeoxyglucose (FDG; ¹⁸F-glucose). FDG is fluorinated glucose molecule and has been shown to be provide noninvasive assessment of metabolic activity in liver, muscles and adipose tissue (45). There is differential metabolism of FDG in the liver, muscles, visceral adipose tissue (VAT) and subcutaneous adipose tissue (SAT) (45, 46). Furthermore, FDG PET has been shown to be an effective tool to assess for insulin resistance (45, 46). Whole body FDG PET MRI is performed in GAST-17 treated subjects before and at 3, 6, and 12 months, and in controls at pre- and 6 months of study only as in this group are not expected. The differential pattern of FDG uptake and metabolism in liver, muscle, VAT and SAT serves as noninvasive markers of insulin resistance. Patient is scanned from skull base to mid-thighs. Quantitative measures of FDG metabolism are calculated as Standard Uptake Value (SUV)=(MBq/g)/(body weight[g]/injected dose [Mbq]). The region of interests (ROI) are 2 cm in diameter and include metabolic activity in the liver (right hepatic lobe, left hepatic lobe, caudate); SAT (L3 level), VAT (omentum), and rectus muscle (45, 47).

### Statistical Analysis

The Dose Escalation Phase of the study uses a 3+3 design with 3 dose levels 15 μg/kg BID, 30 μg/kg BID and 30 μg/kg TID of gastrin. When de-escalation occurs at 30 μg/kg BID, it reduces to a modified dose level 2 at 15 μg/kg TID before further reduced to dose 1. The Treatment Expansion Phase randomizes patients to the treatment and control arm with a 2:1 ratio. Based on the previous research reported by Bokvist el al, the changes of HbA1c from baseline to the end of the 4-week treatment phase was −0.8+/−0.1% in the LY+TT223 (3 mg) group and −0.2+/−0.2% in the placebo group.

### Limitations and Alternative Approaches.

Subjects intolerance to higher gastrin doses.
Inadequate duration of therapy.
Side effects from gastrin intake.
Smaller magnitude of response due to inappropriate patient selection.
Negative imaging results.

### Example 4: Methods for Islet Culture

Day of Isolation (Day 0) Culture Procedure. BSC Preparation: Perform all islet culture procedures in the BSC that has been designated for islet culture and prior to starting procedure, cover the top of the BSC with a sterile drape. Aseptically place the following items into the BSC: T-175 flasks, T-75 flasks, serological pipets, 250 mL conical tubes, and 250 mL conical tube rack. Spray pipet-aids, culture medium and marker with 70% IPA before placing in the BSC. Transfer the 250 mL conical tubes containing the islet fractions onto the 250 mL conical tub rack in the BSC.

Islets: Record Culture Medium Batch # and Expiration date. Indicate if islets will be cultured in flasks or bags. Record the total IEQ for each fraction and the Grand Total IEQ. Record the total IEQ sampled for QC assessment DO. Optionally, additional Fr. 1 islets may be cultured in one T-75 or T-175 flask for non-GMP use. The flask is collected as per the standard Harvesting and Packaging procedure. The Total IEG Cultured is obtained by subtracting the total IEQ for QC Assessment taken from the Grand Total IEQ and recorded.

Islets cultured in Culture Flasks: It is preferred to culture islets in flasks, however, if the culture flasks are not available, bags are an alternative.

TABLE A

| Purity | Number of Flasks | IEQ/Flask | Volume per flask (mL) |
|---|---|---|---|
| High | Total IEQ Cultured ÷ (20,000 | Total IEQ | [IEQ per Flask High × Total |
| ≥70% | IEQ per flask × (% purity ÷ | Cultured ÷ | suspension volume] ÷ Total |
| | 100) | Number of flasks | IEQ Cultured= |
| | | (rounded up) | |
| Medium | Total IEQ Cultured ÷ (10,000 | Total IEQ | [IEQ per Flask Med × Total |
| 40-69% | IEQ per flask × (% purity ÷ | Cultured ÷ | suspension volume] ÷ Total |
| | 100) | Number of flasks | IEQ Cultured= |
| | | (rounded up) | |
| Low | Total IEQ Cultured ÷ (5,000 | Total IEQ | [IEQ per Flask Low × Total |
| <40% | IEQ per flask × (% purity ÷ | Cultured ÷ | suspension volume] ÷ Total |
| | 100) | Number of flasks | IEQ Cultured= |
| | | (rounded up) | |

Calculate the number of T-175 culture flasks required and the volume of tissue to be added to each culture flask, using Table A below. Record calculations and totals on Table 5 of WS-1398.

Record purity, culture temperature, IEQ in culture per fraction, IEQ per flask/bag, number of flask/bag, total mLs per flask/bag, islet appearance, clumping, time islets placed in incubator, and incubator BIS # for each fraction.

Place the required number of flasks into the BSC and inspect them for any damage. Discard any damaged flask.

Transferring the calculated volume of islets to the labeled culture flasks: Add 10 mL of culture medium to the empty flasks. Add the calculated tissue volume (Table 3) from the 250 mL conical tube into the flask and enough Culture Medium for a final volume of 30 mL per flask. Note: Leave the last flask empty. Rinse the 250 mL conical thoroughly with additional culture media to collect the remaining islets and transfer into the last flask. The final volume should be 30 mL. Cap the flasks tightly and gently mix to distribute islet cells evenly. Avoid leaving cells on the neck and sides of the flasks.

Place flasks in 5% CO2 incubator, temperature 22 C to 30 C with 95% humidity. Place the flasks with caps facing towards the incubator door. Record culture flask start time (time islets placed in incubator) and incubator BIS # for each fraction.

Islets Cultured in Bags: Calculate the number of bags needed by dividing the total packed tissue volume of each fraction by 0.2 mL or the desired packed tissue volume. Note: Each bag will contain a final 0.2 mL of packed tissue volume re-suspended in 160 mL of Culture Medium. Place the following items inside the BSC: 1000 mL platelet storage bags, coupler(s), 60 mL syringe, ring stand with rod, 3-prong clamp and serological pipet. Spray the pipet-aid with 70% isopropyl alcohol. Assemble ring stand and attach a 3-prong claim. Secure a 60 mL syringe without plunger to the 3-prong clamp. Remove extraneous tubing from the 1000 mL platelet storage bag using a heat sealer. Insert coupler into the middle port of the bag. Ensure that coupler pinch is open. Disconnect coupler cap and attach to the 60 mL syringe on ring stand. Add 50 mL of Culture Medium into the bag. Add the tissue and remaining Culture Medium for a total volume of 160 mL. Close coupler pinch clamp, disconnect coupler from 60 mL syringe and recap. Prepare label tag(s) with Hu #, Fraction # and date. Attach tag to bag with cable tie and record required information. Place 1-2 bags per tra (no not overlap bags) in 5% C)2 incubator, temperature 22 C to 30 C with 95% humidity.

### Media Change Procedure (Optional):

For clinical use preparations; if islets are to be cultured for >72 hours, the Culture Medium is changed within 12-30 hours of initial culture. Prepare (if necessary) and equilibrate Culture Medium at room temperature before use. Set up the pH meter. Remove flasks or bags from the tissue culture incubator and record date and time. Record the # of minutes the BSC is run before use and the Culture Medium Batch # and expiration date. Record the temperature islets were cultured. Examine each flask or bag for signs of contamination, appearance and clumping. Sings of contamination must be reported immediately for further investigation.

BSC set up for media change: Run the BSC for at least 15 min prior to use. Prior to start of the media change procedure, cover the top of the BSC with a sterile drape. Place the following inside the BSC: Flasks or bags containing the cultured islets, serological pipets, Culture Medium, 250 mL and 50 mL conical tube(s), pipet aid, serological pipettes, 250 mL tube rack(s), marker, and, if needed, culture flasks (T-175 or T-75). If islets are cultured in bags, also place inside the BSC a ring stand and rod, 3-prong clamp, and 60 mL syringe.

Media change: Flasks (Skip if bags are used instead of flasks). Tilt culture flasks at an angle approximately 45 degrees on a tube rack and allow the islets to settle for 10-15 minutes. Remove 20 mL supernatant media from each flask without disturbing the settled islets and pool the supernatant into 250 mL conical tube(s)/Use a marker to label the conical with: Hu #, Fraction # and “Supernatant”. Observe the supernatant to examine the presence of tissue or islet particles. If tissue is detected, centrifuge the supernatant and combine the pooled tissue pellet in a flask. Label the flask with the designated supernatant fraction. Label a 50 mL conical tube for each fraction and take 15-20 mL sample from a supernatant of each fraction into the conical. Measure the pH of the supernatant from each fraction and record. Replenish each flask with 20 mL Culture Meium. Cap the flasks tightly and gently rock to distribute islets cells evenly. Place the culture flasks in a tissue culture incubator at 22-30 C in 95% humidity and 5% CO2 until ready for connection. Record the date and time, re-culture temperature and incubator BIS

Media Change: Bags (Refer to above if flasks are used). To change the media from bags, let tissues settle for 10 minutes by hanging the bag vertically from a right stand. Attach a 60 mL syringe to the coupler that is connected to the bag. Without disturbing the islets, use the syringe to remove 100 mL of the supernatant from each back and fraction separately and place it into 250 conical tube(s). Label the conical with Hu #, Fraction #, and “Supernatant”. Centrifuge the supernatant. After centrifugation, take 15-20 mL sample from the supernatant of each fraction into a 50 mL conical tube and measure the pH. Record. Combine the pellets of the same fractions, re-suspend it in 20 mL of culture media and infuse into the same bag of the corresponding fraction using the coupler and syringe attached. Rinse the same conical with 80 mL of culture media and infuse into the same bag of the corresponding fraction using the coupler and syringe attached. Tightly cap the coupler attached to the bag and mix gently to distribute islet cells evenly. Record the volume of culture media replenished. Replace all the culture bags in a tissue culture incubator at 30 C in 95% humidity and 5% CO2 until ready for collection. Record the date, time, and incubator BIS #.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### REFERENCES

1. Sapra A, Bhandari P. Diabetes Mellitus. StatPearls. Treasure Island (FL)2020.
2. Economic Costs of Diabetes in the U.S. in 2017. Diabetes Care. 2018.
3. Liu Y, Sayam S, Shao X, Wang K, Zheng S, Li Y, Wang L. Prevalence of and Trends in Diabetes Among Veterans, United States, 2005-2014. Prev Chronic Dis. 2017; 14:E135. doi: 10.5888/pcd14.170230. PubMedPMID: 29240552; PMCID: PMC5737977.
4. Ames G M, Duke M R, Moore R S, Cunradi C B. The Impact of Occupational Culture on Drinking Behavior of Young Adults in the U.S. Navy. Journal of Mixed Methods Research. 2009; 3(2):129-50. doi: 10.1177/1558689808328534.
5. Morden E, Oster M, O'Brien C P. Substance use disorders in the U S Armed Forces: National Academies Press; 2013.
6. Seal K H, Cohen G, Waldrop A, Cohen B E, Maguen S, Ren L. Substance use disorders in Iraq and Afghanistan veterans in V A healthcare, 2001-2010: Implications for screening, diagnosis and treatment. Drug Alcohol Depend. 2011; 116(1-3):93-101. doi: 10.1016/j.drugalcdep.2010.11.027. PubMed PMID: 21277712.
7. Wagner T H, Harris K M, Federman B, Dai L, Luna Y, Humphreys K. Prevalence of substance use disorders among veterans and comparable nonveterans from the National Survey on Drug Use and Health. Psychological Services. 2007; 4(3):149.
8. Yang A L, Vadhavkar S, Singh G, Omary M B. Epidemiology of alcohol-related liver and pancreatic disease in the United States. Archives of internal medicine. 2008; 168(6):649-56.
9. Zasimowicz E, Wolszczak B, Zasimowicz B. [Influence of ethyl alcohol on diabetes pathogenesis type]. Pol Merkur Lekarski. 2014; 36(213):212-4. PubMed PMID: 24779223.
10. Wolszczak B, Zasimowicz E, Juchniewicz J. [The effect of ethanol on the endocrynic system]. Pol Merkur Lekarski. 2014; 36(211):45-7. PubMed PMID: 24645579.
11. Livingstone S J, Levin D, Looker H C, Lindsay R S, Wild S H, Joss N, Leese G, Leslie P, McCrimmon R J, Metcalfe W, McKnight J A, Morris A D, Pearson D W, Petrie J R, Philip S, Sattar N A, Traynor J P, Colhoun H M. Estimated life expectancy in a Scottish cohort with type 1 diabetes, 2008-2010. Jama. 2015; 313(1):3744. Epub 2015/01/07. doi: 10.1001/jama.2014.16425. PubMed PMID: 25562264; PMCID: PMC4426486.
12. Foster N C, Beck R W, Miller K M, Clements M A, Rickels M R, DiMeglio L A, Maahs D M, Tamborlane W V, Bergenstal R, Smith E, Olson B A, Garg S K. State of Type 1 Diabetes Management and Outcomes from the T1D Exchange in 2016-2018. Diabetes Technol Ther. 2019; 21(2):66-72. doi: 10.1089/dia.2018.0384. PubMed PMID: 30657336; PMCID: PMC7061293.
13. Hering B J, Kandaswamy R, Ansite J D, Eckman P M, Nakano M, Sawada T, Matsumoto I, Ihm S H, Zhang H J, Parkey J, Hunter D W, Sutherland D E. Single-donor, marginal-dose islet transplantation in patients with type 1 diabetes. Jama. 2005:830-5. PubMed PMID: 15713772.
14. Hering B J, Clarke W R, Bridges N D, Eggerman T L, Alejandro R, Bellin M D, Chaloner K, Czarniecki C W, Goldstein J S, Hunsicker L G, Kaufman D B, Korsgren O, Larsen C P, Luo X, Markmann J F, Naji A, Oberholzer J, Posselt A M, Rickels M R, Ricordi C, Robien M A, Senior P A, Shapiro A M, Stock P G, Turgeon N A, Clinical Islet Transplantation C. Phase 3 Trial of Transplantation of Human Islets in Type 1 Diabetes Complicated by Severe Hypoglycemia. Diabetes Care. 2016; 39(7):1230-40. doi: 10.2337/dc15-1988. PubMed PMID: 27208344; PMCID: PMC5317236.
15. Narimiya M. [Symptomatology of diabetes mellitus]. Nihon Rinsho. 1990; 48 Suppl:635-42. PubMed PMID: 2086945.
16. Barton F B. Update from the Collaborative Islet Transplant Registry (CITR) for the CITR Investigators. 2010 Rachmiel Levine Diabetes and Obesity Symposium; Mar. 14-17, 2010; Las Vegas, NV 2010.
17. Barton F B, Rickels M R, Alejandro R, Hering B J, Wease S, Naziruddin B, Oberholzer J, Odorico J S, Garfinkel M R, Levy M, Pattou F, Berney T, Secchi A, Messinger S, Senior P A, Maffi P, Posselt A, Stock P G, Kaufman D B, Luo X, Kandeel F, Cagliero E, Turgeon N A, Witkowski P, Naji A, O'Connell P J, Greenbaum C, Kudva Y C, Brayman K L, Aull M J, Larsen C, Kay T W, Fernandez L A, Vantyghem M C, Bellin M, Shapiro A M. Improvement in outcomes of clinical islet transplantation: 1999-2010. Diabetes Care. 2012; 35(7):1436-45. doi: 10.2337/dc12-0063. PubMed PMID: 22723582; PMCID: PMC3379615.
18. Braghi S, Maffi P, Bertuzzi F, Monti L D, Secchi A, Pozza G, Bonifacio E, Bosi E, editors. Islet autoantibody response induced by islet allotransplantation in patients with type 1 diabetes. The ADA 60th Scientific Sessions; 2000 Jun. 9-13, 2000; San Antonia, Texas.
19. Bartlett S T, Chin T, Dirden B, Quereshi A, Hadley G. Inclusion of peripancreatic lymph node cells prevents recurrent autoimmune destruction of islet transplants: evidence of donor chimerism. Surgery. 1995; 118:3927. PubMed PMID: 7638756.
20. Uchikoshi F, Yang Z D, Rostami S, Yokoi Y, Capocci P, Barker C F, Naji A. Prevention of autoimmune recurrence and rejection by adenovirus-mediated CTLA4Ig gene transfer to the pancreatic graft in B B rat. Diabetes. 1999; 48:652-7. PubMed PMID: 10078573.
21. Huurman V A, Hilbrands R, Pinkse G G, Gillard P, Duinkerken G, van de Linde P, van der Meer-Prins P M, Versteeg-van der Voort Maarschalk M F, Verbeeck K, Alizadeh B Z, Mathieu C, Gorus F K, Roelen D L, Claas F H, Keymeulen B, Pipeleers D G, Roep B O. Cellular islet autoimmunity associates with clinical outcome of islet cell transplantation. PLoS One. 2008; 3(6):e2435. Epub 2008/06/19. doi: 10.1371/journal.pone.0002435. PubMed PMID: 18560516; PMCID: 2426735.
22. Tamura K, Fujimura T, Tsutsumi T, Nakamura K, Ogawa T, Atumaru C, Hirano Y, Ohara K, Ohtsuka K, Shimomura K, et al. Transcriptional inhibition of insulin by FK506 and possible involvement of FK506 binding protein-12 in pancreatic beta-cell. Transplantation. 1995; 59:1606-13. PubMed PMID: 7539960.
23. Noguchi N, Takasawa S, Nata K, Tohgo A, Kato I, Ikehata F, Yonekura H, Okamoto H. Cyclic ADP-ribose binds to FK506-binding protein 12.6 to release Ca2+ from islet microsomes. J Biol Chem. 1997; 272:31336.
24. Masharani U, Karam J H, German M S. Pancreatic hormones and diabetes mellitus. In: Greenspan F S, Gardner D G, editors. Basic and Clinical Endocrinology, 7th edition. New York: McGraw-Hill Companies; 2004. p. 658-746.
25. Montana E, Bonner-Weir S, Weir G C. Beta cell mass and growth after syngeneic islet cell transplantation in normal and streptozocin diabetic C57BL/6 mice. J Clin Invest. 1993; 91(3):780-7. doi: 10.1172/JCI116297. PubMed PMID: 8450059; PMCID: PMC288028.
26. Davalli A M, Scaglia L, Zangen D H, Hollister J, Bonner-Weir S, Weir G C. Vulnerability of islets in the immediate posttransplantation period. Dynamic changes in structure and function. Diabetes. 1996; 45(9):1161-7. PubMed PMID: 8772716.
27. Eich T, Eriksson O, Sundin A, Estrada S, Brandhorst D, Brandhorst H, Langstrom B, Nilsson B, Korsgren O, Lundgren T. Positron emission tomography: a real-time tool to quantify early islet engraftment in a preclinical large anima model. Transplantation. 2007; 84(7):893-8. doi: 10.1097/01.tp.0000284730.86567.9f. PubMed PMID: 17984843.
28. Farney A C, Xenos E, Sutherland D E, Widmer M, Stephanian E, Field M J, Kaufman D B, Stevens R B, Blazar B, Platt J, et al. Inhibition of pancreatic islet beta cell function by tumor necrosis factor is blocked by a soluble tumor necrosis factor receptor. Transplant Proc. 1993; 25(1 Pt 2):865-6. PubMed PMID: 8382881.
29. Ryan E A, Paty B W, Senior P A, Bigam D, Alfadhli E, Kneteman N M, Lakey J R, Shapiro A M. Five-year follow-up after clinical islet transplantation. Diabetes. 2005; 54(7):2060-9. PubMed PMID: 15983207.
30. Froud T, Ricordi C, Baidal D A, Hafiz M M, Ponte G, Cure P, Pileggi A, Poggioli R, Ichii H, Khan A, Ferreira J V, Pugliese A, Esquenazi V V, Kenyon N S, Alejandro R. Islet transplantation in type 1 diabetes mellitus using cultured islets and steroid-free immunosuppression: Miami experience. Am J Transplant. 2005; 5(8):2037-46. PubMed PMID: 15996257.
31. CITR Annual Report 2005: Draft
32. Song S Y, Gannon M, Washington M K, Scoggins C R, Meszoely I M, Goldenring J R, Marino C R, Sandgren E P, Coffey R J, Jr., Wright C V, Leach S D. Expansion of Pdx1-expressing pancreatic epithelium and islet neogenesis in transgenic mice overexpressing transforming growth factor alpha. Gastroenterology. 1999; 117:1416-26.
33. Cras-Meneur C, Elghazi L, Czernichow P, Scharfmann R. Epidermal growth factor increases undifferentiated pancreatic embryonic cells in vitro: a balance between proliferation and differentiation. Diabetes. 2001; 50:1571-9.
34. Xu G, Stoffers D A, Habener J F, Bonner-Weir S. Exendin-4 stimulates both beta-cell replication and neogenesis, resulting in increased beta-cell mass and improved glucose tolerance in diabetic rats. Diabetes. 1999; 48:2270-6.
35. Tourrel C, Bailbe D, Meile M J, Kergoat M, Portha B. Glucagon-like peptide-1 and exendin-4 stimulate beta-cell neogenesis in streptozotocin-treated newborn rats resulting in persistently improved glucose homeostasis at adult age. Diabetes. 2001; 50:1562-70.
36. Wang T C, Bonner-Weir S, Oates P S, Chulak M, Simon B, Merlino G T, Schmidt E V, Brand S J. Pancreatic gastrin stimulates islet differentiation of transforming growth factor alpha-induced ductular precursor cells. J Clin Invest. 1993; 92:1349-56.
37. Rooman I, Lardon J, Bouwens L. Gastrin stimulates beta-cell neogenesis and increases islet mass from transdifferentiated but not from normal exocrine pancreas tissue. Diabetes. 2002; 51:686-90.
38. Suarez-Pinzon W L, Lakey J R, Brand S J, Rabinovitch A. Combination therapy with epidermal growth factor and gastrin induces neogenesis of human islet {beta}-cells from pancreatic duct cells and an increase in functional {beta}-cell mass. J Clin Endocrinol Metab. 2005; 90:3401-9. PubMed PMID: 15769977.
39. Suarez-Pinzon W L, Yan Y, Power R, Brand S J, Rabinovitch A. Combination therapy with epidermal growth factor and gastrin increases beta-cell mass and reverses hyperglycemia in diabetic NOD mice. Diabetes. 2005; 54:2596-601. PubMed PMID: 16123347.
40. TransitionTherapeuticsInc. News Release—Transition Therapeutics provides update on lead diabetes regenerative therapy E1-I.N.T. clinical trials. 2005.
41. Bardhan K D, Bishop A E, Polak J M, Romanska H M, Rowland A, Thompson M, Morris P, Schaefer-Preuss S, Luehmann R, McCaldin B. Pantoprazole in severe acid-peptic disease: the effectiveness and safety of 5 years' continuous treatment. Dig Liver Dis. 2005; 37(1):10-22. PubMed PMID: 15702854.
42. Hove K D, Faerch K, Bodvarsdottir T B, Karlsen A E, Petersen J S, Vaag A. Treatment with a proton pump inhibitor improves glycaemic control in type 2 diabetic patients—a retrospective analysis. Diabetes Res Clin Pract. 2010; 90(3):e72-4. doi: 10.1016/j.diabres.2010.09.007. PubMed PMID: 20888658.
43. Boj-Carceller D. Proton pump inhibitors: impact on glucose metabolism. Endocrine. 2013; 43(1):22-32. doi: 10.1007/s12020-012-9755-3. PubMed PMID: 22886351.
44. Gomez-Izquierdo J C, Yu O H Y. The Influence of Proton-Pump Inhibitors on Glycemic Control: A Systematic Review of the Literature and a Meta-Analysis. Can J Diabetes. 2017; 41(4):351-61. doi: 10.1016/j.jcjd.2016.11.004. PubMed PMID: 28373033.
45. Hove K D, Brons C, Faerch K, Lund S S, Petersen J S, Karlsen A E, Rossing P, Rehfeld J F, Vaag A. Effects of 12 weeks' treatment with a proton pump inhibitor on insulin secretion, glucose metabolism and markers of cardiovascular risk in patients with type 2 diabetes: a randomised double-blind prospective placebo-controlled study. Diabetologia. 2013; 56(1):22-30. doi: 10.1007/s00125-012-2714-y. PubMed PMID: 23011351.
46. Zhang M, Lin Q, Qi T, Wang T, Chen C C, Riggs A D, Zeng D. Growth factors and medium hyperglycemia induce Sox9+ ductal cell differentiation into beta cells in mice with reversal of diabetes. Proc Natl Acad Sci USA. 2016; 113(3):650-5. doi: 10.1073/pnas.1524200113. PubMed PMID: 26733677; PMCID: PMC4725504.
47. Dahan T, Ziv O, Horwitz E, Zemmour H, Lavi J, Swisa A, Leibowitz G, Ashcroft F M, In't Veld P, Glaser B, Dor Y. Pancreatic beta-Cells Express the Fetal Islet Hormone Gastrin in Rodent and Human Diabetes. Diabetes. 2017; 66(2):426-36. doi: 10.2337/db16-0641. PubMed PMID: 27864307; PMCID: PMC5248995.
48. Griffin K J, Thompson P A, Gottschalk M, Kyllo J H, Rabinovitch A. Combination therapy with sitagliptin and lansoprazole in patients with recent-onset type 1 diabetes (REPAIR-T1D): 12-month results of a multicentre, randomised, placebo-controlled, phase 2 trial. The lancet Diabetes & endocrinology. 2014. Epub 2014/07/07. doi: 10.1016/S2213-8587(14)70115-9. PubMed PMID: 24997559.
49. Wu Z, Liu S, Hassink M, Nair I, Park R, Li L, Todorov I, Fox J M, Li Z, Shively J E, Conti P S, Kandeel F. Development and evaluation of 18F-TTCO-Cys40-Exendin-4: a PET probe for imaging transplanted islets. Journal of nuclear medicine: official publication, Society of Nuclear Medicine. 2013; 54(2):244-51. Epub 2013/01/09. doi: 10.2967/jnumed.112.109694. PubMed PMID: 23297075.
50. Drucker D J. Enhancing incretin action for the treatment of type 2 diabetes. Diabetes Care. 2003; 26(10):2929-40. PubMed PMID: 14514604.
51. Suarez-Pinzon W L, Rabinovitch A. Combination therapy with a dipeptidyl peptidase-4 inhibitor and a proton pump inhibitor induces beta-cell neogenesis from adult human pancreatic duct cells implanted in immunodeficient mice. Cell transplantation. 2011; 20(9):1343-9. Epub 2011/03/15. doi: 10.3727/096368910X557263. PubMed PMID: 21396168.
52. Pettus J, Hirsch I, Edelman S. GLP-1 agonists in type 1 diabetes. Clin Immunol. 2013; 149(3):317-23. Epub 2013/05/07. doi: 10.1016/j.clim.2013.04.006. PubMed PMID: 23643354.
53. Unger J. Rationale use of GLP-1 receptor agonists in patients with type 1 diabetes. Current diabetes reports. 2013; 13(5):663-8. Epub 2013/08/21. doi: 10.1007/s11892-013-0404-x. PubMed PMID: 23955813.
54. Xu G, Kaneto H, Lopez-Avalos M D, Weir G C, Bonner-Weir S. GLP-1/exendin-4 facilitates beta-cell neogenesis in rat and human pancreatic ducts. Diabetes Res Clin Pract. 2006; 73(1):107-10. Epub 2006/01/13. doi: 10.1016/j.diabres.2005.11.007. PubMed PMID: 16406191.
55. Levetan C, Pozzilli P, Jovanovic L, Schatz D. Proposal for generating new beta cells in a muted immune environment for type 1 diabetes. Diabetes/metabolism research and reviews. 2013; 29(8):604-6. Epub 2013/07/16. doi: 10.1002/dmrr.2435. PubMed PMID: 23853103.
56. Ellis S L, Moser E G, Snell-Bergeon J K, Rodionova A S, Hazenfield R M, Garg S K. Effect of sitagliptin on glucose control in adult patients with Type 1 diabetes: a pilot, double-blind, randomized, crossover trial. Diabetic medicine: a journal of the British Diabetic Association. 2011; 28(10):1176-81. Epub 2011/09/20. doi: 10.1111/j.1464-5491.2011.03331.x. PubMed PMID: 21923696.
57. Lind M, Jendle J, Torffvit O, Lager I. Glucagon-like peptide 1 (GLP-1) analogue combined with insulin reduces HbA1c and weight with low risk of hypoglycemia and high treatment satisfaction. Primary care diabetes. 2012; 6(1):41-6. Epub 2011/10/22. doi: 10.1016/j.pcd.2011.09.002. PubMed PMID: 22015237.
58. Ludvigsson J. The latest pharmacotherapy options for type 1 diabetes. Expert opinion on pharmacotherapy. 2014; 15(1):37-49. Epub 2013/12/03. doi: 10.1517/14656566.2014.855197. PubMed PMID: 24289822.
59. Price J D, Linder G, Li W P, Zimmermann B, Rother K I, Malek R, Alattar M, Tarbell K V. Effects of short-term sitagliptin treatment on immune parameters in healthy individuals, a randomized placebo-controlled study. Clinical and experimental immunology. 2013; 174(1):120-8. Epub 2013/05/29. doi: 10.1111/cei.12144. PubMed PMID: 23711188; PMCID: 3784219.
60. Patel V, Joharapurkar A, Dhanesha N, Kshirsagar S, Detroja J, Patel K, Gandhi T, Bahekar R, Jain M. Combination of omeprazole with GLP-1 agonist therapy improves insulin sensitivity and antioxidant activity in liver in type 1 diabetic mice. Pharmacological reports: P R. 2013; 65(4):927-36. Epub 2013/10/23. PubMed PMID: 24145087.
61. Garg S K, Moser E G, Bode B W, Klaff L J, Hiatt W R, Beatson C, Snell-Bergeon J K. Effect of sitagliptin on post-prandial glucagon and GLP-1 levels in patients with type 1 diabetes: investigator-initiated, double-blind, randomized, placebo-controlled trial. Endocrine practice: official journal of the American College of Endocrinology and the American Association of Clinical Endocrinologists. 2013; 19(1):19-28. Epub 2012/11/29. doi: 10.4158/EP12100.OR. PubMed PMID: 23186950.
62. Ghazi T, Rink L, Sherr J L, Herold K C. Acute metabolic effects of exenatide in patients with type 1 diabetes with and without residual insulin to oral and intravenous glucose challenges. Diabetes Care. 2014; 37(1):2106. Epub 2013/08/14. doi: 10.2337/dc13-1169. PubMed PMID: 23939544.
63. de Koning E J, Bonner-Weir S, Rabelink T J. Preservation of beta-cell function by targeting beta-cell mass. Trends in pharmacological sciences. 2008; 29(4):218-27. Epub 2008/03/25. doi: 10.1016/j.tips.2008.02.001. PubMed PMID: 18359095.
64. Lenz A, Lenz G, Ku H T, Ferreri K, Kandeel F. Islets from human donors with higher but not lower hemoglobin A1c levels respond to gastrin treatment in vitro. PLoS One. 2019; 14(8):e0221456. doi: 10.1371/journal.pone.0221456. PubMed PMID: 31430329; PMCID: PMC6701795.
65. Suarez-Pinzon W L, Power R F, Yan Y, Wasserfall C, Atkinson M, Rabinovitch A. Combination Therapy with Glucagon-Like Peptide-1 and Gastrin Restores Normoglycemia in Diabetic NOD Mice. Diabetes. 2008. PubMed PMID: 18835930.
66. Suarez-Pinzon W L, Lakey J R, Rabinovitch A. Combination therapy with glucagon-like peptide-1 and gastrin induces beta-cell neogenesis from pancreatic duct cells in human islets transplanted in immunodeficient diabetic mice. Cell transplantation. 2008; 17(6):631-40. PubMed PMID: 18819251.
67. DCCT. Epidemiology of severe hypoglycemia in the diabetes control and complications trial. The DCCT Research Group. Am J Med. 1991; 90(4):450-9. Epub 1991/04/01. PubMed PMID: 2012085.
68. Levy J, Matthews D, Hermans M. Correct homeostasis model assessment (HOMA) evaluation uses the computer program. Diabetes Care. 1998; 21:2191-2.
69. Hostens K, Ling Z, Van Schravendijk C, Pipeleers D. Prolonged exposure of human beta-cells to high glucose increases their release of proinsulin during acute stimulation with glucose or arginine. J Clin Endocrinol Metab. 1999; 84(4):1386-90. Epub 1999/04/13. PubMed PMID: 10199783.
70. Hostens K, Pavlovic D, Zambre Y, Ling Z, Van Schravendijk C, Eizirik D L, Pipeleers D G. Exposure of human islets to cytokines can result in disproportionately elevated proinsulin release. J Clin Invest. 1999; 104(1):67-72. Epub 1999/07/07. doi: 10.1172/JC16438. PubMed PMID: 10393700; PMCID: 408405.
71. Klimek A M, Soukhatcheva G, Thompson D M, Warnock G L, Salehi M, Rilo H, D′Alessio D, Meneilly G S, Panagiotopoulos C, Verchere C B. Impaired proinsulin processing is a characteristic of transplanted islets. Am J Transplant. 2009; 9(9):2119-25. Epub 2009/08/27. doi: AJT2740 10.1111/j.1600-6143.2009.02740.x. PubMed PMID: 19706025.
72. McDonald C G, Ryan E A, Paty B W, Senior P A, Marshall S M, Lakey J R, Shapiro A M. Cross-sectional and prospective association between proinsulin secretion and graft function after clinical islet transplantation. Transplantation. 2004; 78(6):934-7. Epub 2004/09/24. doi: 00007890-200409270-00023 [pii]. PubMed PMID: 15385817.
73. Rickels M R, Mueller R, Markmann J F, Naji A. Effect of glucagon-like peptide-1 on beta- and alpha-cell function in isolated islet and whole pancreas transplant recipients. J Clin Endocrinol Metab. 2009; 94(1):1819. Epub 2008/10/30. doi: jc.2008-1806 [pii]10.1210/jc.2008-1806. PubMed PMID: 18957498; PMCID: 2630873.
74. Rickels M R, Naji A. Proinsulin processing and transplanted islets. Am J Transplant. 10(6):1495. Epub 2010/03/30. doi: AJT3069 [pii]10.1111/j.1600-6143.2010.03069.x. PubMed PMID: 20346066.
75. Peyser T A, Balo A K, Buckingham B A, Hirsch I B, Garcia A. Glycemic Variability Percentage: A Novel Method for Assessing Glycemic Variability from Continuous Glucose Monitor Data. Diabetes Technol Ther. 2018; 20(1):6-16. doi: 10.1089/dia.2017.0187. PubMed PMID: 29227755; PMCID: PMC5846572.
76. Ryan E A, Shandro T, Green K, Paty B W, Senior P A, Bigam D, Shapiro A M, Vantyghem M C. Assessment of the severity of hypoglycemia and glycemic lability in type 1 diabetic subjects undergoing islet transplantation. Diabetes. 2004; 53(4):955-62. PubMed PMID: 15047610.
77. Clarke W L, Cox D J, Gonder-Frederick L A, Julian D, Schlundt D, Polonsky W. Reduced awareness of hypoglycemia in adults with IDDM. A prospective study of hypoglycemic frequency and associated symptoms. Diabetes Care. 1995; 18(4):517-22. Epub 1995/04/01. PubMed PMID: 7497862.
78. Service F J, Molnar G D, Rosevear J W, Ackerman E, Gatewood L C, Taylor W F. Mean amplitude of glycemic excursions, a measure of diabetic instability. Diabetes. 1970; 19(9):644-55. Epub 1970/09/01. PubMed PMID: 5469118.
79. Ryan E A, Paty B W, Senior P A, Lakey J R, Bigam D, Shapiro A M. Beta-score: an assessment of beta-cell function after islet transplantation. Diabetes Care. 2005; 28:343-7. PubMed PMID: 15677790.
80. Orr C, Stratton J, Rao I, Al-Sayed M, Smith C, El-Shahawy M, Dafoe D, Mullen Y, Al-Abdullah I, Kandeel F. Quantifying Insulin Therapy Requirements to Preserve Islet Graft Function Following Islet Transplantation. Cell Transplant. 2016; 25(1):83-95. doi: 10.3727/096368915X687958. PubMed PMID: 25853639.
81. Poggioli R, Faradji R N, Ponte G, Betancourt A, Messinger S, Baidal D A, Froud T, Ricordi C, Alejandro R. Quality of life after islet transplantation. Am J Transplant. 2006; 6:371-8. PubMed PMID: 16426323.
82. Barshes N R, Vanatta J M, Mote A, Lee T C, Schock A P, Balkrishnan R, Brunicardi F C, Goss J A. Health-related quality of life after pancreatic islet transplantation: a longitudinal study. Transplantation. 2005; 79:1727-30. PubMed PMID: 15973176.
83. Foster E D, Bridges N D, Feurer I D, Eggerman T L, Hunsicker L G, Alejandro R, Clinical Islet Transplantation C. Improved Health-Related Quality of Life in a Phase 3 Islet Transplantation Trial in Type 1 Diabetes Complicated by Severe Hypoglycemia. Diabetes Care. 2018; 41(5):1001-8. doi: 10.2337/dc17-1779. PubMed PMID: 29563196; PMCID: PMC5911786.
84. Kowalski R, Post D, Schneider M C, Britz J, Thomas J, Deierhoi M, Lobashevsky A, Redfield R, Schweitzer E, Heredia A, Reardon E, Davis C, Bentlejewski C, Fung J, Shapiro R, Zeevi A. Immune cell function testing: an adjunct to therapeutic drug monitoring in transplant patient management. Clin Transplant. 2003; 17:7788. PubMed PMID: 12709071.
85. Liu Z, Sun Y K, Xi Y P, Maffei A, Reed E, Harris P, Suciu-Foca N. Contribution of direct and indirect recognition pathways to T cell alloreactivity. J Exp Med. 1993; 177:1643-50. PubMed PMID: 7684431.
86. Liu Z, Colovai A I, Tugulea S, Reed E F, Fisher P E, Mancini D, Rose E A, Cortesini R, Michler R E, Suciu-Foca N. Indirect recognition of donor HLA-D R peptides in organ allograft rejection. J Clin Invest. 1996; 98:1150-7.
87. Liu Z, Harris P E, Colovai A I, Reed E F, Maffei A, Suciu-Foca N. Indirect recognition of donor MHC Class II antigens in human transplantation. Clin Immunol Immunopathol. 1996; 78:228-35. PubMed PMID: 8605698.
88. Molajoni E R, Cinti P, Orlandini A, Molajoni J, Tugulea S, Ho E, Liu Z, Suciu-Foca N, Cortesini R. Mechanism of liver allograft rejection: the indirect recognition pathway. Hum Immunol. 1997; 53:57-63. PubMed PMID: 9127148.
89. Ciubotariu R, Liu Z, Colovai A I, Ho E, Itescu S, Ravalli S, Hardy M A, Cortesini R, Rose E A, Suciu-Foca N. Persistent allopeptide reactivity and epitope spreading in chronic rejection of organ allografts. J Clin Invest. 1998; 101:398-405. PubMed PMID: 9435312.
90. Vella J P, Spadafora-Ferreira M, Murphy B, Alexander S I, Harmon W, Carpenter C B, Sayegh M H. Indirect allorecognition of major histocompatibility complex allopeptides in human renal transplant recipients with chronic graft dysfunction. Transplantation. 1997; 64:795-800.
91. Hornick P I, Mason P D, Baker R J, Hernandez-Fuentes M, Frasca L, Lombardi G, Taylor K, Weng L, Rose M L, Yacoub M H, Batchelor R, Lechler R I. Significant frequencies of T cells with indirect anti-donor specificity in heart graft recipients with chronic rejection. Circulation. 2000; 101:2405.
92. SivaSai K S, Smith M A, Poindexter N J, Sundaresan S R, Trulock E P, Lynch J P, Cooper J D, Patterson G A, Mohanakumar T. Indirect recognition of donor HLA class I peptides in lung transplant recipients with bronchiolitis obliterans syndrome. Transplantation. 1999; 67:1094.
93. Korin Y D, Lee C, Gjertson D W, Wilkinson A H, Pham T P, Danovitch G M, Gritsch H A, Reed E F. A novel flow assay for the detection of cytokine secreting alloreactive T cells: application to immune monitoring. Hum Immunol. 2005; 66(11):1110-24. PubMed PMID: 16571412.
94. Roep B O, Stobbe I, Duinkerken G, van Rood J J, Lernmark A, Keymeulen B, Pipeleers D, Claas F H, de Vries R R. Auto- and allo immune reactivity to human islet allografts transplanted into type 1 diabetic patients. Diabetes. 1999; 48:484-90.
95. Jaeger C, Hering B J, Dyrberg T, Federlin K, Bretzel R G. Islet cell antibodies and glutamic acid decarboxylase antibodies in patients with insulin-dependent diabetes mellitus undergoing kidney and islet-after-kidney transplantation. Transplantation. 1996; 62:424-6.
96. Jaeger C, Brendel M, Hering B, Eckhard M, Bretzel R. Progressive islet graft failure occurs significantly earlier in autoantibody-positive than in autoantibody-negative IDDM recipients of intrahepatic islet allografts. Diabetes. 1997; 46:1907-10.
97. Laybutt D R, Glandt M, Xu G, Ahn Y B, Trivedi N, Bonner-Weir S, Weir G C. Critical reduction in beta-cell mass results in two distinct outcomes over time. Adaptation with impaired glucose tolerance or decompensated diabetes. J Biol Chem. 2003; 278(5):2997-3005. PubMed PMID: 12438314.
98. Laybutt D R, Hawkins Y C, Lock J, Lebet J, Sharma A, Bonner-Weir S, Weir G C. Influence of diabetes on the loss of beta cell differentiation after islet transplantation in rats. Diabetologia. 2007. PubMed PMID: 17641871.
99. Ritz-Laser B, Oberholzer J, Toso C, Brulhart M C, Zakrzewska K, Ris F, Bucher P, Morel P, Philippe J. Molecular detection of circulating beta-cells after islet transplantation. Diabetes. 2002; 51:557-61.
100. Tsui N B, Ng E K, Lo Y M. Stability of endogenous and added RNA in blood specimens, serum, and plasma. Clin Chem. 2002; 48:1647-53. PubMed PMID: 12324479.
101. Interewicz B, Olszewski W L, Sikora M, Gorna K, Kolakowska B. DNA released from ischemic and rejecting organs as an indicator of graft cellular damage. Ann Transplant. 2004; 9:42-5. PubMed PMID: 15478917.
102. Interewicz B, Olszewski W L, Stanislawska J, Sikora M, Durlik M, Maksymowicz M. DNA released from rejecting organs is an indicator of the degree of graft cellular damage. Transplant Proc. 2005; 37:98-101. PubMed PMID: 15808560.
103. Berney T, Ritz-Laser B, Mamin A, Toso C, Brulhart M C, Bucher P, Aeschlimann R, Andres A, Morel P, Philippe J. Detection of circulating beta cells in the peripheral blood after human islet transplantation predicts deterioration of metabolic control. Transplantation. 2004; 78(2 (Suppl 1)):177.
104. Liu Q, Sommer S S. Detection of extremely rare alleles by bidirectional pyrophosphorolysis-activated polymerization allele-specific amplification (Bi-PAP-A): measurement of mutation load in mammalian tissues. Biotechniques. 2004; 36:156-66. PubMed PMID: 14740499.
105. Husseiny M I, Kuroda A, Kaye A N, Nair I, Kandeel F, Ferreri K. Development of a quantitative methylation-specific polymerase chain reaction method for monitoring beta cell death in type 1 diabetes. PLoS One. 2012; 7(10):e47942. doi: 10.1371/journal.pone.0047942. PubMed PMID: 23144715; PMCID: 3483298.
106. Husseiny M I, Kaye A, Zebadua E, Kandeel F, Ferreri K. Tissue-specific methylation of human insulin gene and PCR assay for monitoring beta cell death. PLos ONE. 2014; 9(4):e94591.
107. Aslamy A, Oh E, Ahn M, Moin A S M, Chang M, Duncan M, Hacker-Stratton J, El-Shahawy M, Kandeel F, DiMeglio L A, Thurmond D C. Exocytosis Protein DOC2B as a Biomarker of Type 1 Diabetes. J Clin Endocrinol Metab. 2018; 103(5):1966-76. doi: 10.1210/jc.2017-02492. PubMed PMID: 29506054; PMCID: PMC6276681.
108. Tiwari J L, Schneider B, Barton F, Anderson S A. Islet cell transplantation in type 1 diabetes: an analysis of efficacy outcomes and considerations for trial designs. Am J Transplant. 2012; 12(7):1898-907. doi: 10.1111/j.1600-6143.2012.04038.x. PubMed PMID: 22486926.

### Additional Embodiments

Embodiment 1. A method of treating diabetes in a subject in need thereof, the method comprising administering a dosage of gastrin-treated human islet cells to the subject, wherein the dosage comprises less than 9,000 IEQ/kg of islet cells.

Embodiment 2. The method of embodiment 1, wherein the dosage comprises less than 8,000 IEQ/kg of islet cells.

Embodiment 3. The method of embodiment 1 or 2, wherein the dosage comprises less than 7,000 IEQ/kg of islet cells.

Embodiment 4. The method of any one of embodiments 1 to 3, wherein the dosage comprises less than 6,000 IEQ/kg of islet cells.

Embodiment 5. The method of any one of embodiments 1 to 4, wherein the dosage comprises less than 5,000 IEQ/kg of islet cells.

Embodiment 6. The method of any one of embodiments 1 to 5, wherein the gastrin-treated human islet cells are treated with gastrin 17.

Embodiment 7. The method of any of any one of embodiments 1 to 6, wherein the human islet cells are not obtained from the subject.

Embodiment 8. The method of any one of embodiments 1 to 7, wherein the gastrin-treated human islet cells are obtained by a method comprising:
culturing islet cells from a donor;
contacting the culture with gastrin; and
harvesting the islet cells.

Embodiment 9. The method of any one of embodiments 1 to 8, further comprising administering to the subject gastrin.

Embodiment 10. The method of embodiment 9, wherein the gastrin is administered to the subject prior to administration of the dosage of the gastrin-treated human islet cells.

Embodiment 11. The method of embodiment 9, wherein the gastrin is administered to the subject after the administration of the dosage of gastrin-treated human islet cells.

Embodiment 12. The method of embodiment 9 or 11, wherein the gastrin is administered to the subject about two days after the administration of the dosage of gastrin-treated human islet cells.

Embodiment 13. The method of any one of embodiments 9, 11 and 12, wherein the gastrin is administered to the subject at least one time per day for about 30 days.

Embodiment 14. The method of any one of embodiments 9 to 13, wherein the gastrin is administered to the subject two times per day.

Embodiment 15. The method of any one of embodiments 9 and 11 to 14, wherein the gastrin is administered to the subject about two days after the administration of the dosage of gastrin-treated human islet cells for two times per day for about 30 days.

Embodiment 16. The method of any of embodiments 9 to 15, wherein the gastrin is administered to the subject at a dosage of about 15 μg/kg.

Embodiment 17. The method of any of embodiments 9 to 16, wherein the gastrin is administered to the subject subcutaneously.

Embodiment 18. The method of any of embodiments 9 to 17, further comprising administering a second dosage of gastrin to the subject.

Embodiment 19. The method of embodiment 18, wherein the second dosage of gastrin is administered to the subject about six months after administering the dosage of gastrin-treated human islet cells.

Embodiment 20. The method of embodiment 19, wherein the second dosage of gastrin is administered to the subject is at least one time per day for about 30 days.

Embodiment 21. The method of any one of embodiments 18 to 20, wherein the second dosage of gastrin is administered to the subject two times per day.

Embodiment 22. The method of any one of embodiments 1 to 21, further comprising administering to the subject a proton pump inhibitor and a DPP-4 inhibitor.

Embodiment 23. The method of embodiment 22, wherein the proton pump inhibitor is Esomeprazole.

Embodiment 24. The method of embodiment 22, wherein the DPP-4 inhibitor is Sitagliptin.

Embodiment 25. The method of any one of embodiments 1 to 24, wherein the subject has Type 1 diabetes.

Embodiment 26. The method of any one of embodiments 1 to 24, wherein the subject has Type 2 diabetes.

Embodiment 27. The method of any one of the above embodiments, wherein the subject is rendered insulin-independent.

Embodiment 28. A kit for preparing gastrin-treated islet cells, the kit comprising a gastrin composition and instructions for use.

Embodiment 29. A method of treating diabetes in a subject in need thereof, the method comprising administering a dosage of gastrin and a dosage of islet cells to the subject.

Embodiment 30. The method of embodiment 29, wherein the islet cells are pre-treated with gastrin.

Embodiment 31. The method of embodiment 29 or 30, wherein the dosage of islet cells comprises less than 9,000 IEQ/kg of islet cells.

Embodiment 32. The method of embodiment 29, wherein the gastrin is administered prior to, concurrently with, or after the administering of the dosage of islet cells.

Embodiment 33. The method of embodiment 32, wherein the gastrin is administered prior to the administering of the dosage of islet cells.

Embodiment 34. The method of embodiment 33, wherein the gastrin is administered about one week, two weeks, three weeks, one month, or longer, prior to the administering of the dosage of islet cells.

Embodiment 35. The method of any one of embodiments 32 to 34, wherein the gastrin is administered continuously until at least one week, two weeks, three weeks, one month, two months, three months, four months, or longer, after the administering of the dosage of islet cell.

Embodiment 36. The method of embodiment 32, wherein the gastrin is administered to the subject after the administration of the dosage of islet cells.

Embodiment 37. The method of embodiment 36, wherein the gastrin is administered to the subject about one day, two days, three days, four days, five days, one week, two weeks, three weeks, one month, or longer, after the administration of the dosage of islet cells.

Embodiment 38. The method of embodiment 36 or 37, wherein the gastrin is administered continuously until at least one week, two weeks, three weeks, one month, two months, three months, four months, or longer, after the administering of the dosage of islet cell.

Embodiment 39. The method of embodiment 32, wherein the gastrin is administered to the subject about two weeks prior to the administration of the dosage of islet cells, wherein the gastrin is continuously administered for two times per day, once per day, once per two days, once per three days, once per one week, or less frequent, for about one month, two months, three months, or longer.

Embodiment 40. The method of embodiment 32, wherein the gastrin is administered to the subject about two days after the administration of the dosage of islet cells, wherein the gastrin is continuously administered for two times per day, once per day, once per two days, once per three days, once per one week, or less frequent, for about one month, two months, three months, or longer.

Embodiment 41. The method of any one of embodiments 29 to 40, wherein the gastrin is administered to the subject once per day or two times per day.

Embodiment 42. The method of embodiment 41, wherein the gastrin is administered to the subject at a daily dosage of about 15 μg/kg to about 30 μg/kg, about 20 μg/kg to about 40 μg/kg, about 25 μg/kg to about 50 μg/kg, about 30 μg/kg to about 60 μg/kg, about 40 μg/kg to about 70 μg/kg, about 50 μg/kg to about 80 μg/kg, or more.

Embodiment 43. The method of any one of embodiments 29 to 42, wherein the gastrin is administered to the subject subcutaneously.

Embodiment 44. The method of embodiment 29, further comprising administering a second dosage of gastrin to the subject.

Embodiment 45. The method of embodiment 44, wherein the second dosage of gastrin is administered to the subject about six months after administering the dosage of gastrin-treated human islet cells.

Embodiment 46. The method of embodiment 44 or 45, wherein the second dosage of gastrin is administered to the subject is at least one time per day for about 30 days.

Embodiment 47. The method of any one of embodiments 44 to 46, wherein the second dosage of gastrin is administered to the subject two times per day.

Embodiment 48. The method of any one of embodiments 29 to 47, further comprising administering to the subject a proton pump inhibitor and a DPP-4 inhibitor.

Embodiment 49. The method of embodiment 48, wherein the proton pump inhibitor is Esomeprazole.

Embodiment 50. The method of embodiment 48, wherein the DPP-4 inhibitor is Sitagliptin.

Embodiment 51. The method of any one of embodiments 29 to 50, wherein the subject has Type 1 diabetes.

Embodiment 52. The method of any one of embodiments 29 to 50, wherein the subject has Type 2 diabetes.

Embodiment 53. The method of any one of embodiments 29 to 52, wherein the subject is rendered insulin-independent.

1. A method of treating diabetes in a subject in need thereof, said method comprising administering a dosage of gastrin-treated human islet cells to said subject, wherein said dosage comprises less than 9,000 IEQ/kg of islet cells.

2. The method of claim 1, wherein said dosage comprises less than 8,000 IEQ/kg of islet cells.

3. The method of claim 1, wherein said dosage comprises less than 7,000 IEQ/kg of islet cells.

4. The method of claim 1, wherein said dosage comprises less than 6,000 IEQ/kg of islet cells.

5. The method of claim 1, wherein said dosage comprises less than 5,000 IEQ/kg of islet cells.

6. The method of claim 1, wherein said gastrin-treated human islet cells are treated with gastrin 17.

7. The method of claim 1, wherein said human islet cells are not obtained from said subject.

8. The method of claim 1, wherein said gastrin-treated human islet cells are obtained by a method comprising:
culturing islet cells from a donor;
contacting said culture with gastrin; and,
harvesting said islet cells.

9. The method of claim 1, further comprising administering to said subject gastrin.

10. The method of claim 9, wherein said gastrin is administered to the subject prior to administration of said dosage of said gastrin-treated human islet cells.

11. The method of claim 9, wherein said gastrin is administered to the subject after said administration of said dosage of gastrin-treated human islet cells.

12. The method of claim 9, wherein said gastrin is administered to the subject about two days after said administration of said dosage of gastrin-treated human islet cells.

13. The method of claim 9, wherein said gastrin is administered to said subject at least one time per day for about 30 days.

14. The method of claim 9, wherein said gastrin is administered to said subject two times per day.

15. The method of claim 9, wherein said gastrin is administered to said subject about two days after said administration of said dosage of gastrin-treated human islet cells for two times per day for about 30 days.

16. The method of claim 9, wherein said gastrin is administered to said subject at a dosage of about 15 μg/kg.

17. The method of claim 9, wherein said gastrin is administered to said subject subcutaneously.

18. The method of claim 9, further comprising administering a second dosage of gastrin to said subject.

19. The method of claim 18, wherein said second dosage of gastrin is administered to said subject about six months after administering said dosage of gastrin-treated human islet cells.

20. The method of claim 19, wherein said second dosage of gastrin is administered to said subject is at least one time per day for about 30 days.

21. The method of claim 18, wherein said second dosage of gastrin is administered to said subject two times per day.

22. The method of claim 1, further comprising administering to said subject a proton pump inhibitor and a DPP-4 inhibitor.

23. The method of claim 22, wherein said proton pump inhibitor is Esomeprazole.

24. The method of claim 22, wherein said DPP-4 inhibitor is Sitagliptin.

25. The method of claim 1, wherein said subject has Type 1 diabetes.

26. The method of claim 1, wherein said subject has Type 2 diabetes.

27. The method of claim 1, wherein said subject is rendered insulin-independent.

28. A kit for preparing gastrin-treated islet cells, the kit comprising a gastrin composition and instructions for use.

29. A method of treating diabetes in a subject in need thereof, said method comprising administering a dosage of gastrin and a dosage of islet cells to said subject.

30. The method of claim 29, wherein the islet cells are pre-treated with gastrin.

31. The method of claim 29, wherein said dosage of islet cells comprises less than 9,000 IEQ/kg of islet cells.

32. The method of claim 29, wherein said gastrin is administered prior to, concurrently with, or after the administering of the dosage of islet cells.

33. The method of claim 32, wherein said gastrin is administered prior to the administering of the dosage of islet cells.

34. The method of claim 33, wherein said gastrin is administered about one week, two weeks, three weeks, one month, or longer, prior to the administering of the dosage of islet cells.

35. The method of claim 32, wherein said gastrin is administered continuously until at least one week, two weeks, three weeks, one month, two months, three months, four months, or longer, after the administering of the dosage of islet cell.

36. The method of claim 32, wherein said gastrin is administered to the subject after said administration of said dosage of islet cells.

37. The method of claim 36, wherein said gastrin is administered to the subject about one day, two days, three days, four days, five days, one week, two weeks, three weeks, one month, or longer, after said administration of said dosage of islet cells.

38. The method of claim 36, wherein said gastrin is administered continuously until at least one week, two weeks, three weeks, one month, two months, three months, four months, or longer, after the administering of the dosage of islet cell.

39. The method of claim 32, wherein said gastrin is administered to said subject about two weeks prior to said administration of said dosage of islet cells, wherein said gastrin is continuously administered for two times per day, once per day, once per two days, once per three days, once per one week, or less frequent, for about one month, two months, three months, or longer.

40. The method of claim 32, wherein said gastrin is administered to said subject about two days after said administration of said dosage of islet cells, wherein said gastrin is continuously administered for two times per day, once per day, once per two days, once per three days, once per one week, or less frequent, for about one month, two months, three months, or longer.

41. The method of claim 29, wherein said gastrin is administered to said subject once per day or two times per day.

42. The method of 41, wherein said gastrin is administered to said subject at a daily dosage of about 15 μg/kg to about 30 μg/kg, about 20 μg/kg to about 40 μg/kg, about 25 g/kg to about 50 μg/kg, about 30 μg/kg to about 60 μg/kg, about 40 μg/kg to about 70 μg/kg, about 50 μg/kg to about 80 μg/kg, or more.

43. The method of claim 29, wherein said gastrin is administered to said subject subcutaneously.

44. The method of claim 29, further comprising administering a second dosage of gastrin to said subject.

45. The method of claim 44, wherein said second dosage of gastrin is administered to said subject about six months after administering said dosage of gastrin-treated human islet cells.

46. The method of claim 44, wherein said second dosage of gastrin is administered to said subject is at least one time per day for about 30 days.

47. The method of claim 44, wherein said second dosage of gastrin is administered to said subject two times per day.

48. The method of claim 29, further comprising administering to said subject a proton pump inhibitor and a DPP-4 inhibitor.

49. The method of claim 48, wherein said proton pump inhibitor is Esomeprazole.

50. The method of claim 48, wherein said DPP-4 inhibitor is Sitagliptin.

51. The method of claim 29, wherein said subject has Type 1 diabetes.

52. The method of claim 29, wherein said subject has Type 2 diabetes.

53. The method of claim 29, wherein said subject is rendered insulin-independent.


SEQUENCE LISTING (2 sequences)

SEQ ID NO: 1 - protein, 17 aa, Artificial Sequence
  Chemically Synthesized
  MISC_FEATURE (1)..(1): Wherein X at position 1 is pyroglutamic acid
XGPWLEEEEEAYGWMDF

SEQ ID NO: 2 - protein, 101 aa, Homo sapiens
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN